SEQUENCE LISTING (107 sequences)

SEQ ID NO: 1 - protein, 118 aa, synthetic construct
  REGION 1..118: note = Description of Artificial Sequence: Synthetic polypeptide
EVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSS

SEQ ID NO: 2 - protein, 7 aa, synthetic construct
  REGION 1..7: note = Description of Artificial Sequence: Synthetic peptide
GFTFSNS

SEQ ID NO: 3 - protein, 6 aa, synthetic construct
  REGION 1..6: note = Description of Artificial Sequence: Synthetic peptide
SDGGLY

SEQ ID NO: 4 - protein, 9 aa, synthetic construct
  REGION 1..9: note = Description of Artificial Sequence: Synthetic peptide
QGVRPFFDY

SEQ ID NO: 5 - protein, 25 aa, synthetic construct
  REGION 1..25: note = Description of Artificial Sequence: Synthetic peptide
EVQMVESGGDLVKPGGSLKLSCAAS

SEQ ID NO: 6 - protein, 19 aa, synthetic construct
  REGION 1..19: note = Description of Artificial Sequence: Synthetic peptide
GMSWVRQTPDKRLEWVASI

SEQ ID NO: 7 - protein, 19 aa, synthetic construct
  REGION 1..19: note = Description of Artificial Sequence: Synthetic peptide
THYPDSVKGEDTAIYYCAR

SEQ ID NO: 8 - protein, 11 aa, synthetic construct
  REGION 1..11: note = Description of Artificial Sequence: Synthetic peptide
WGQGTTLTVSS

SEQ ID NO: 9 - protein, 107 aa, synthetic construct
  REGION 1..107: note = Description of Artificial Sequence: Synthetic polypeptide
DIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIK

SEQ ID NO: 10 - protein, 107 aa, synthetic construct
  REGION 1..107: note = Description of Artificial Sequence: Synthetic polypeptide
DIQMTQSPASLSASVGETVTMTCRASENLYSYLAWYQQKQGKSPQLLVFNAETLPEGVPSRFSGTGSGTHFSLRINSLQPEDFGSYYCQHHYVIPWTFGGGTKLEIK

SEQ ID NO: 11 - protein, 11 aa, synthetic construct
  REGION 1..11: note = Description of Artificial Sequence: Synthetic peptide
RASENIYSYLA

SEQ ID NO: 12 - protein, 11 aa, synthetic construct
  REGION 1..11: note = Description of Artificial Sequence: Synthetic peptide
RASENLYSYLA

SEQ ID NO: 13 - protein, 7 aa, synthetic construct
  REGION 1..7: note = Description of Artificial Sequence: Synthetic peptide
NAETLPE

SEQ ID NO: 14 - protein, 9 aa, synthetic construct
  REGION 1..9: note = Description of Artificial Sequence: Synthetic peptide
QHHYVIPWT

SEQ ID NO: 15 - protein, 23 aa, synthetic construct
  REGION 1..23: note = Description of Artificial Sequence: Synthetic peptide
DIQMTQSPASLSASVGETVTMTC

SEQ ID NO: 16 - protein, 15 aa, synthetic construct
  REGION 1..15: note = Description of Artificial Sequence: Synthetic peptide
WYQQKQGKSPQLLVF

SEQ ID NO: 17 - protein, 32 aa, synthetic construct
  REGION 1..32: note = Description of Artificial Sequence: Synthetic peptide
GVPSRFSGTGSGTHFSLRINSLQPEDFGSYYC

SEQ ID NO: 18 - protein, 10 aa, synthetic construct
  REGION 1..10: note = Description of Artificial Sequence: Synthetic peptide
FGGGTKLEIK

SEQ ID NO: 19 - skipped

SEQ ID NO: 20 - protein, 6 aa, synthetic construct
  REGION 1..6: note = Description of Artificial Sequence: Synthetic peptide
GGSGGS

SEQ ID NO: 21 - protein, 9 aa, synthetic construct
  REGION 1..9: note = Description of Artificial Sequence: Synthetic peptide
GGSGGSGGS

SEQ ID NO: 22 - protein, 12 aa, synthetic construct
  REGION 1..12: note = Description of Artificial Sequence: Synthetic peptide
GGSGGSGGSGGS

SEQ ID NO: 23 - protein, 15 aa, synthetic construct
  REGION 1..15: note = Description of Artificial Sequence: Synthetic peptide
GGSGGSGGSGGSGGS

SEQ ID NO: 24 - protein, 4 aa, synthetic construct
  REGION 1..4: note = Description of Artificial Sequence: Synthetic peptide
GGGS

SEQ ID NO: 25 - protein, 8 aa, synthetic construct
  REGION 1..8: note = Description of Artificial Sequence: Synthetic peptide
GGGSGGGS

SEQ ID NO: 26 - protein, 12 aa, synthetic construct
  REGION 1..12: note = Description of Artificial Sequence: Synthetic peptide
GGGSGGGSGGGS

SEQ ID NO: 27 - protein, 16 aa, synthetic construct
  REGION 1..16: note = Description of Artificial Sequence: Synthetic peptide
GGGSGGGSGGGSGGGS

SEQ ID NO: 28 - protein, 20 aa, synthetic construct
  REGION 1..20: note = Description of Artificial Sequence: Synthetic peptide
GGGSGGGSGGGSGGGSGGGS

SEQ ID NO: 29 - protein, 5 aa, synthetic construct
  REGION 1..5: note = Description of Artificial Sequence: Synthetic peptide
GGGGS

SEQ ID NO: 30 - protein, 10 aa, synthetic construct
  REGION 1..10: note = Description of Artificial Sequence: Synthetic peptide
GGGGSGGGGS

SEQ ID NO: 31 - protein, 15 aa, synthetic construct
  REGION 1..15: note = Description of Artificial Sequence: Synthetic peptide
GGGGSGGGGSGGGGS

SEQ ID NO: 32 - protein, 20 aa, synthetic construct
  REGION 1..20: note = Description of Artificial Sequence: Synthetic peptide
GGGGSGGGGSGGGGSGGGGS

SEQ ID NO: 33 - protein, 25 aa, synthetic construct
  REGION 1..25: note = Description of Artificial Sequence: Synthetic peptide
GGGGSGGGGSGGGGSGGGGSGGGGS

SEQ ID NO: 34 - protein, 18 aa, synthetic construct
  REGION 1..18: note = Description of Artificial Sequence: Synthetic peptide
GSTSGSGKPGSGEGSTKG

SEQ ID NO: 35 - protein, 20 aa, synthetic construct
  REGION 1..20: note = Description of Artificial Sequence: Synthetic peptide
EAAAKEAAAKEAAAKEAAAK

SEQ ID NO: 36 - protein, 21 aa, synthetic construct
  REGION 1..21: note = Description of Artificial Sequence: Synthetic peptide
IYIWAPLAGTCGVLLLSLVIT

SEQ ID NO: 37 - protein, 27 aa, synthetic construct
  REGION 1..27: note = Description of Artificial Sequence: Synthetic peptide
IYIWAPLAGTCGVLLLSLVITLYCNHR

SEQ ID NO: 38 - protein, 28 aa, synthetic construct
  REGION 1..28: note = Description of Artificial Sequence: Synthetic peptide
IYIWAPLAGTCGVLLLSLVITLYCNHRN

SEQ ID NO: 39 - protein, 45 aa, synthetic construct
  REGION 1..45: note = Description of Artificial Sequence: Synthetic polypeptide
TTTPAPRPPTPAPTIALQPLSLRPEACRPAAGGAVHTRGLDFACD

SEQ ID NO: 40 - protein, 42 aa, synthetic construct
  REGION 1..42: note = Description of Artificial Sequence: Synthetic polypeptide
AAAIEVMYPPPYLDNEKSNGTIIHVKGKHLCPSPLFPGPSKP

SEQ ID NO: 41 - protein, 12 aa, synthetic construct
  REGION 1..12: note = Description of Artificial Sequence: Synthetic peptide
ESKYGPPCPSCP

SEQ ID NO: 42 - protein, 12 aa, synthetic construct
  REGION 1..12: note = Description of Artificial Sequence: Synthetic peptide
ESKYGPPAPSAP

SEQ ID NO: 43 - protein, 12 aa, synthetic construct
  REGION 1..12: note = Description of Artificial Sequence: Synthetic peptide
ESKYGPPCPPCP

SEQ ID NO: 44 - protein, 12 aa, synthetic construct
  REGION 1..12: note = Description of Artificial Sequence: Synthetic peptide
EPKSCDKTHTCP

SEQ ID NO: 45 - protein, 86 aa, synthetic construct
  REGION 1..86: note = Description of Artificial Sequence: Synthetic polypeptide
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 46 - protein, 132 aa, synthetic construct
  REGION 1..132: note = Description of Artificial Sequence: Synthetic polypeptide
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 47 - protein, 34 aa, synthetic construct
  REGION 1..34: note = Description of Artificial Sequence: Synthetic polypeptide
ACPTGLYTHSGECCKACNLGEGVAQPCGANQTVC

SEQ ID NO: 48 - protein, 20 aa, synthetic construct
  REGION 1..20: note = Description of Artificial Sequence: Synthetic peptide
AVGQDTQEVIVVPHSLPFKV

SEQ ID NO: 49 - protein, 45 aa, synthetic construct; identical to SEQ ID NO: 39
  REGION 1..45: note = Description of Artificial Sequence: Synthetic polypeptide

SEQ ID NO: 50 - protein, 66 aa, synthetic construct
  REGION 1..66: note = Description of Artificial Sequence: Synthetic polypeptide
ALSNSIMYFSHFVPVFLPAKPTTTPAPRPPTPAPTIASQPLSLRPEACRPAAGGAVHTRGLDFACD

SEQ ID NO: 51 - protein, 55 aa, synthetic construct
  REGION 1..55: note = Description of Artificial Sequence: Synthetic polypeptide
FVPVFLPAKPTTTPAPRPPTPAPTIALQPLSLRPEACRPAAGGAVHTRGLDFACD

SEQ ID NO: 52 - DNA, 135 bp, synthetic construct
  misc_feature 1..135: note = Description of Artificial Sequence: Synthetic oligonucleotide
  source 1..135: mol_type = other DNA
accacaactcctgctcctagacctcctacaccagctcctacaatcgccctgcagccactgtctctgaggccagaagcttgtagacctgctgcaggcggagccgtgcatacaagaggactggatttcgcctgcgac

SEQ ID NO: 53 - DNA, 126 bp, synthetic construct
  misc_feature 1..126: note = Description of Artificial Sequence: Synthetic oligonucleotide
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 54 - DNA, 36 bp, synthetic construct
  misc_feature 1..36: note = Description of Artificial Sequence: Synthetic oligonucleotide
  source 1..36: mol_type = other DNA
gaaagcaagtacggtccaccttgccctagctgtccg

SEQ ID NO: 55 - DNA, 36 bp, synthetic construct
  misc_feature 1..36: note = Description of Artificial Sequence: Synthetic oligonucleotide
  source 1..36: mol_type = other DNA
gaatccaagtacggccccccagcgcctagtgcccca

SEQ ID NO: 56 - DNA, 36 bp, synthetic construct
  misc_feature 1..36: note = Description of Artificial Sequence: Synthetic oligonucleotide
  source 1..36: mol_type = other DNA
gaatctaaatatggcccgccatgcccgccttgccca

SEQ ID NO: 57 - DNA, 36 bp, synthetic construct
  misc_feature 1..36: note = Description of Artificial Sequence: Synthetic oligonucleotide
  source 1..36: mol_type = other DNA
gaaccgaagtcttgtgataaaactcatacgtgcccg

SEQ ID NO: 58 - DNA, 258 bp, synthetic construct
  misc_feature 1..258: note = Description of Artificial Sequence: Synthetic polynucleotide
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 59 - DNA, 396 bp, synthetic construct
  misc_feature 1..396: note = Description of Artificial Sequence: Synthetic polynucleotide
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 60 - DNA, 102 bp, synthetic construct
  misc_feature 1..102: note = Description of Artificial Sequence: Synthetic polynucleotide
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 61 - DNA, 60 bp, synthetic construct
  misc_feature 1..60: note = Description of Artificial Sequence: Synthetic oligonucleotide
  source 1..60: mol_type = other DNA
gctgtgggccaggacacgcaggaggtcatcgtggtgccacactccttgccctttaaggtg

SEQ ID NO: 62 - protein, 12 aa, synthetic construct
  REGION 1..12: note = Description of Artificial Sequence: Synthetic peptide
GGCKJSGGCKJS

SEQ ID NO: 63 - protein, 504 aa, synthetic construct
  REGION 1..504: note = Description of Artificial Sequence: Synthetic polypeptide
MALPVTALLLPLALLLHAARPEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGGGGSGGGGSGGGGSDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGKPIPNPLLGLDSTNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 504 aa, synthetic construct
  REGION 1..504: note = Description of Artificial Sequence: Synthetic polypeptide
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGGGGSGGGGSGGGGSEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 507 aa, synthetic construct
  REGION 1..507: note = Description of Artificial Sequence: Synthetic polypeptide
MALPVTALLLPLALLLHAARPEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGSTSGSGKPGSGEGSTKGDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGKPIPNPLLGLDSTNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 507 aa, synthetic construct
  REGION 1..507: note = Description of Artificial Sequence: Synthetic polypeptide
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 494 aa, synthetic construct
  REGION 1..494: note = Description of Artificial Sequence: Synthetic polypeptide
MALPVTALLLPLALLLHAARPEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGGGGSDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGKPIPNPLLGLDSTNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 494 aa, synthetic construct
  REGION 1..494: note = Description of Artificial Sequence: Synthetic polypeptide
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGGGGSEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 240 aa, synthetic construct
  REGION 1..240: note = Description of Artificial Sequence: Synthetic polypeptide
EVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGGGGSGGGGSGGGGSDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIK

SEQ ID NO: 70 - protein, 240 aa, synthetic construct
  REGION 1..240: note = Description of Artificial Sequence: Synthetic polypeptide
DIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGGGGSGGGGSGGGGSEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSS

SEQ ID NO: 71 - protein, 243 aa, synthetic construct
  REGION 1..243: note = Description of Artificial Sequence: Synthetic polypeptide
EVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGSTSGSGKPGSGEGSTKGDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIK

SEQ ID NO: 72 - protein, 243 aa, synthetic construct
  REGION 1..243: note = Description of Artificial Sequence: Synthetic polypeptide
DIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSS

SEQ ID NO: 73 - protein, 230 aa, synthetic construct
  REGION 1..230: note = Description of Artificial Sequence: Synthetic polypeptide
EVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGGGGSDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIK

SEQ ID NO: 74 - protein, 230 aa, synthetic construct
  REGION 1..230: note = Description of Artificial Sequence: Synthetic polypeptide
DIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGGGGSEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSS

SEQ ID NO: 75 - DNA, 720 bp, synthetic construct
  misc_feature 1..720: note = Description of Artificial Sequence: Synthetic polynucleotide
  source 1..720: mol_type = other DNA
gaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctggcggaggcggatcaggtggcggaggaagtggcggcggaggatctgacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaag

SEQ ID NO: 76 - DNA, 720 bp, synthetic construct
  misc_feature 1..720: note = Description of Artificial Sequence: Synthetic polynucleotide
  source 1..720: mol_type = other DNA
gacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcggaggcggatcaggtggcggaggaagtggcggcggaggatctgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatct

SEQ ID NO: 77 - DNA, 729 bp, synthetic construct
  misc_feature 1..729: note = Description of Artificial Sequence: Synthetic polynucleotide
  source 1..729: mol_type = other DNA
gaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctggcagcacaagcggctctggaaaacctggatctggcgagggctctaccaagggcgacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaag

SEQ ID NO: 78 - DNA, 729 bp, synthetic construct
  misc_feature 1..729: note = Description of Artificial Sequence: Synthetic polynucleotide
  source 1..729: mol_type = other DNA
gacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatct

SEQ ID NO: 79 - DNA, 690 bp, synthetic construct
  misc_feature 1..690: note = Description of Artificial Sequence: Synthetic polynucleotide
  source 1..690: mol_type = other DNA
gaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctggtggcggaggcagcgacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaag

SEQ ID NO: 80 - DNA, 690 bp, synthetic construct
  misc_feature 1..690: note = Description of Artificial Sequence: Synthetic polynucleotide
  source 1..690: mol_type = other DNA
gacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggtggcggaggcagcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatct

SEQ ID NO: 81 - DNA, 720 bp, synthetic construct
  misc_feature 1..720: note = Description of Artificial Sequence: Synthetic polynucleotide
  source 1..720: mol_type = other DNA
gaggtgcagatggtggaaagcggcggagatctggtgaagcctggcggcagcctgaagctgtcttgtgccgcttctggctttaccttcagcaacagcggcatgagctgggtcagacagacccctgataagcggctggaatgggtggccagcatcagcgacggcggactgtacacccactacccagacagcgtgaaaggcagattcacaatcagccgggacaacggcaagtccaccctgtatctgcagatgtctagcctgagaagcgaggacaccgccatctactactgcgccagacaaggcgtgcgccccttcttcgactactggggccagggaacaacactcaccgtgtcctccggcggaggcggatcaggtggcggaggaagtggcggcggaggatctgatatccagatgacccagagccctgcttctctgagcgccagcgtgggcgagacagtgaccatgacctgtagagcctccgagaatatctacagctacctggcctggtaccagcagaaacagggcaagagcccccagctgctggtctttaacgccgagacactgcctgaaggcgtgccatctagattctccggcaccggcagcggcacacacttcagcctccggatcaacagcctgcaacctgaggacttcggatcttattactgccagcaccactacgtgatcccctggaccttcggcggaggcaccaagctggaaatcaag

SEQ ID NO: 82 - DNA, 720 bp, synthetic construct
  misc_feature 1..720: note = Description of Artificial Sequence: Synthetic polynucleotide
  source 1..720: mol_type = other DNA
gatatccagatgacccagagccctgcttctctgagcgccagcgtgggcgagacagtgaccatgacctgtagagcctccgagaatatctacagctacctggcctggtaccagcagaaacagggcaagagcccccagctgctggtctttaacgccgagacactgcctgaaggcgtgccatctagattctccggcaccggcagcggcacacacttcagcctccggatcaacagcctgcaacctgaggacttcggatcttattactgccagcaccactacgtgatcccctggaccttcggcggaggcaccaagctggaaatcaagggcggaggcggatcaggtggcggaggaagtggcggcggaggatctgaggtgcagatggtggaaagcggcggagatctggtgaagcctggcggcagcctgaagctgtcttgtgccgcttctggctttaccttcagcaacagcggcatgagctgggtcagacagacccctgataagcggctggaatgggtggccagcatcagcgacggcggactgtacacccactacccagacagcgtgaaaggcagattcacaatcagccgggacaacggcaagtccaccctgtatctgcagatgtctagcctgagaagcgaggacaccgccatctactactgcgccagacaaggcgtgcgccccttcttcgactactggggccagggaacaacactcaccgtgtcctcc

SEQ ID NO: 83 - DNA, 729 bp, synthetic construct
  misc_feature 1..729: note = Description of Artificial Sequence: Synthetic polynucleotide
  source 1..729: mol_type = other DNA
gaggtgcagatggtggaaagcggcggagatctggtgaagcctggcggcagcctgaagctgtcttgtgccgcttctggctttaccttcagcaacagcggcatgagctgggtcagacagacccctgataagcggctggaatgggtggccagcatcagcgacggcggactgtacacccactacccagacagcgtgaaaggcagattcacaatcagccgggacaacggcaagtccaccctgtatctgcagatgtctagcctgagaagcgaggacaccgccatctactactgcgccagacaaggcgtgcgccccttcttcgactactggggccagggaacaacactcaccgtgtcctccggcagcacaagcggctctggaaaacctggatctggcgagggctctaccaagggcgatatccagatgacccagagccctgcttctctgagcgccagcgtgggcgagacagtgaccatgacctgtagagcctccgagaatatctacagctacctggcctggtaccagcagaaacagggcaagagcccccagctgctggtctttaacgccgagacactgcctgaaggcgtgccatctagattctccggcaccggcagcggcacacacttcagcctccggatcaacagcctgcaacctgaggacttcggatcttattactgccagcaccactacgtgatcccctggaccttcggcggaggcaccaagctggaaatcaag

SEQ ID NO: 84 - DNA, 729 bp, synthetic construct
  misc_feature 1..729: note = Description of Artificial Sequence: Synthetic polynucleotide
  source 1..729: mol_type = other DNA
gatatccagatgacccagagccctgcttctctgagcgccagcgtgggcgagacagtgaccatgacctgtagagcctccgagaatatctacagctacctggcctggtaccagcagaaacagggcaagagcccccagctgctggtctttaacgccgagacactgcctgaaggcgtgccatctagattctccggcaccggcagcggcacacacttcagcctccggatcaacagcctgcaacctgaggacttcggatcttattactgccagcaccactacgtgatcccctggaccttcggcggaggcaccaagctggaaatcaagggcagcacaagcggctctggaaaacctggatctggcgagggctctaccaagggcgaggtgcagatggtggaaagcggcggagatctggtgaagcctggcggcagcctgaagctgtcttgtgccgcttctggctttaccttcagcaacagcggcatgagctgggtcagacagacccctgataagcggctggaatgggtggccagcatcagcgacggcggactgtacacccactacccagacagcgtgaaaggcagattcacaatcagccgggacaacggcaagtccaccctgtatctgcagatgtctagcctgagaagcgaggacaccgccatctactactgcgccagacaaggcgtgcgccccttcttcgactactggggccagggaacaacactcaccgtgtcctcc

SEQ ID NO: 85 - DNA, 45 bp, synthetic construct
  misc_feature 1..45: note = Description of Artificial Sequence: Synthetic oligonucleotide
  source 1..45: mol_type = other DNA
ggaggcggaggatctggtggcggaggaagtggcggaggcggttct

SEQ ID NO: 86 - DNA, 75 bp, synthetic construct
  misc_feature 1..75: note = Description of Artificial Sequence: Synthetic oligonucleotide
  source 1..75: mol_type = other DNA
ggtggtggtggcagtggtggcggtggctcaggtggcggcggatcaggcggtggtggttctggcggcggtggatct

SEQ ID NO: 87 - protein, 243 aa, synthetic construct; identical to SEQ ID NO: 72
  REGION 1..243: note = Description of Artificial Sequence: Synthetic polypeptide

SEQ ID NO: 88 - protein, 21 aa, synthetic construct
  REGION 1..21: note = Description of Artificial Sequence: Synthetic peptide
ILIGVSVVFLFCLLLLVLFCL

SEQ ID NO: 89 - protein, 101 aa, synthetic construct
  REGION 1..101: note = Description of Artificial Sequence: Synthetic polypeptide
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 90 - protein, 21 aa, synthetic construct
  REGION 1..21: note = Description of Artificial Sequence: Synthetic peptide
VIGILVAVILLLLLLLLLFLI

SEQ ID NO: 91 - protein, 168 aa, synthetic construct
  REGION 1..168: note = Description of Artificial Sequence: Synthetic polypeptide
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 92 - protein, 19 aa, synthetic construct
  REGION 1..19: note = Description of Artificial Sequence: Synthetic peptide
ILIGTSVVIILFILLFFLL

SEQ ID NO: 93 - protein, 84 aa, synthetic construct
  REGION 1..84: note = Description of Artificial Sequence: Synthetic polypeptide
HRWCSNKKNAAVMDQESAGNRTANSEDSDEQDPQEVTYTQLNHCVFTQRKITRPSQRPKTPPTDIIVYTELPNAESRSKVVSCP

SEQ ID NO: 94 - protein, 19 aa, synthetic construct
  REGION 1..19: note = Description of Artificial Sequence: Synthetic peptide
VAVGLGSCLAILILAICGL

SEQ ID NO: 95 - protein, 141 aa, synthetic construct
  REGION 1..141: note = Description of Artificial Sequence: Synthetic polypeptide
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 96 - protein, 20 aa, synthetic construct
  REGION 1..20: note = Description of Artificial Sequence: Synthetic peptide
IVVGVVCTLLVALLMAALYL

SEQ ID NO: 97 - protein, 111 aa, synthetic construct
  REGION 1..111: note = Description of Artificial Sequence: Synthetic polypeptide
VRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 98 - protein, 20 aa, synthetic construct
  REGION 1..20: note = Description of Artificial Sequence: Synthetic peptide
ILIGTSVVIILFILLLFFLL

SEQ ID NO: 99 - protein, 84 aa, synthetic construct
  REGION 1..84: note = Description of Artificial Sequence: Synthetic polypeptide
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 100 - protein, 45 aa, synthetic construct; identical to SEQ ID NO: 39
  REGION 1..45: note = Description of Artificial Sequence: Synthetic polypeptide

SEQ ID NO: 101 - protein, 5 aa, synthetic construct
  REGION 1..5: note = Description of Artificial Sequence: Synthetic peptide
  REGION >5: note = The entire sequence of amino acids 1-5 can be repeated one or six times.
GGGGS

SEQ ID NO: 102 - protein, 25 aa, synthetic construct
  REGION 1..25: note = Description of Artificial Sequence: Synthetic peptide
GGGGSGGGGSGGGGSGGGGSGGGGS

SEQ ID NO: 103 - protein, 107 aa, synthetic construct; identical to SEQ ID NO: 9
  REGION 1..107: note = Description of Artificial Sequence: Synthetic polypeptide

SEQ ID NO: 104 - protein, 118 aa, synthetic construct; identical to SEQ ID NO: 1
  REGION 1..118: note = Description of Artificial Sequence: Synthetic polypeptide

SEQ ID NO: 105 - protein, 15 aa, synthetic construct
  REGION 1..15: note = Description of Artificial Sequence: Synthetic peptide
EAAAKEAAAKEAAAK

SEQ ID NO: 106 - protein, 5 aa, synthetic construct
  REGION >5: note = The entire sequence of amino acids 1-5 can be repeated one or more times.
GGGGS

SEQ ID NO: 107 - protein, 4 aa, synthetic construct
  REGION >4: note = The entire sequence of amino acids 1-4 can be repeated one or more times.
GGGS

(19) United States
(12) Patent Application Publication
(10) Pub. No.: US 2024/0173410 A1
(43) Pub. Date: May 30, 2024

(54) CHIMERIC RECEPTORS AND METHODS OF USE THEREOF

(71) Applicant: Senti Biosciences, Inc., South San Francisco, CA (US)

(72) Inventors: Alba Gonzalez-Junca, South San Francisco, CA (US); Nicholas Frankel, South San Francisco, CA (US); Assen Boyanov Roguev, South San Francisco, CA (US)

(21) Appl. No.: 18/504,037

(22) Filed: Nov. 7, 2023

Related U.S. Application Data
(63) Continuation of application No. PCT/US2022/028202, filed on May 6, 2022.
(60) Provisional application No. 63/283,147, filed on Nov. 24, 2021.
(60) Provisional application No. 63/185,896, filed on May 7, 2021.

(51) Int. Cl. A61K 39/00; C07K 14/725; C07K 16/28

(52) U.S. Cl. CPC A61K 39/464411; A61K 39/4613; A61K 39/4631; C07K 14/7051; C07K 16/2803; C07K 2317/622; C07K 2319/03

(57) ABSTRACT

Provided herein are chimeric proteins comprising an antigen-binding domain specific for V-Set And Immunoglobulin Domain Containing 2 (VSIG2). Also provided herein are cells, polynucleotides, vectors, compositions, and methods directed to chimeric proteins comprising an antigen-binding domain specific for VSIG2.

### CROSS REFERENCE TO RELATED APPLICATIONS

This application is a continuation of International Application Number PCT/US2022/028202 filed on May 6, 2022, which claims priority to U.S. Provisional Patent Application No. 63/185,896, filed May 7, 2021, and to U.S. Provisional Patent Application No. 63/283,147, filed Nov. 24, 2021, each of which is incorporated herein by reference in its entirety for all purposes.

### SEQUENCE LISTING

The instant application contains a Sequence Listing which has been submitted via Patent Center and is hereby incorporated by reference in its entirety. Said XML copy, created on Feb. 19, 2024, is named STB-033WOC1, and is 152,646 bytes in size.

### BACKGROUND

Chimeric antigen receptor (CAR) based adoptive cell therapies used to redirect the specificity and function of immunoresponsive cells, such as T cells, have shown efficacy in patients with lymphoid malignancies (Pule et al., Nat. Med. (14):1264-1270 (2008); Maude et al., N Engl J Med. (371):1507-17 (2014); Brentjens et al., Sci Transl Med. (5):177ra38 (2013)). CAR T cells have been shown to induce complete remission in patients with CD19-expressing malignancies for whom chemotherapies have led to drug resistance and tumor progression. The success of CD19 CAR therapy provides optimism for treating other malignancies, such as solid tumors.

One challenge to developing CAR therapy for solid tumors is the lack of suitable targets. The ability to identify appropriate CAR targets is important to effectively targeting and treating the tumor without damaging normal cells that express the same target antigen. Thus, there remains a need for CAR-based solid tumor therapies that target tumor cells without targeting normal cells or tissues.

### SUMMARY

In order to meet the above-described needs, the present disclosure relates to a chimeric protein including an antigen-binding domain specific for VSIG2 (VSIG2).

In one aspect, provided herein is a chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region, wherein
(a) the VH comprises:
a heavy chain complementarity determining region 1 (CDR-H1) having the amino acid sequence of GFTFSNS (SEQ ID NO:2),
a heavy chain complementarity determining region 2 (CDR-H2) having the amino acid sequence of SDGGLY (SEQ ID NO:3), and
a heavy chain complementarity determining region 3 (CDR-H3) having the amino acid sequence of QGVRPFFDY (SEQ ID NO:4), and the VL comprises:
a light chain complementarity determining region 1 (CDR-L1) having the amino acid sequence of RASENIYSYLA (SEQ ID NO: 11) or RASENLYSYLA (SEQ ID NO:12),
a light chain complementarity determining region 2 (CDR-L2) having the amino acid sequence of NAETLPE (SEQ ID NO:13), and
a light chain complementarity determining region 3 (CDR-L3) having the amino acid sequence of QHHYVIPWT (SEQ ID NO:14); or
(b) the VH comprises:
a heavy chain complementarity determining region 1 (CDR-H1) contained within the VH region amino acid sequence of SEQ ID NO: 1, a heavy chain complementarity determining region 2 (CDR-H2) contained within the VH region amino acid sequence of SEQ ID NO: 1, and a heavy chain complementarity determining region 3 (CDR-H3) contained within the VH region amino acid sequence of SEQ ID NO: 1, and
the VL comprises:
a light chain complementarity determining region 1 (CDR-L1) are contained within the VL region amino acid sequence of SEQ ID NO: 9, a light chain complementarity determining region 2 (CDR-L2) are contained within the VL region amino acid sequence of SEQ ID NO: 9, and a light chain complementarity determining region 3 (CDR-L3) are contained within the VL region amino acid sequence of SEQ ID NO: 9; or
(c) the VH comprises:
a heavy chain complementarity determining region 1 (CDR-H1) contained within the VH region amino acid sequence of SEQ ID NO: 1, a heavy chain complementarity determining region 2 (CDR-H2) contained within the VH region amino acid sequence of SEQ ID NO: 1, and a heavy chain complementarity determining region 3 (CDR-H3) contained within the VH region amino acid sequence of SEQ ID NO: 1, and
the VL comprises:
a light chain complementarity determining region 1 (CDR-L1) are contained within the VL region amino acid sequence of SEQ ID NO: 10, a light chain complementarity determining region 2 (CDR-L2) are contained within the VL region amino acid sequence of SEQ ID NO:10, and a light chain complementarity determining region 3 (CDR-L3) are contained within the VL region amino acid sequence of SEQ ID NO: 10, and
optionally the amino acid sequences of the CDR-H1, the CDR-H2, the CDR-H3, the CDR-L1, the CDR-L2, and the CDR-L3 of the reference antibody are defined based on the Kabat or Chothia numbering scheme.

In some embodiments, the VH region comprises the amino acid sequence of SEQ ID NO: 1. In some embodiments, the VL region comprises the amino acid sequence of SEQ ID NO: 9, or the VL region comprises the amino acid sequence of SEQ ID NO: 10.

In some embodiments, the antigen-binding domain comprises a single chain variable fragment (scFv), optionally the VH and VL of the scFv are separated by a peptide linker, optionally the antigen-binding domain comprises the structure VH-L-VL or VL-L-VH, where VH is the heavy chain variable domain, L is the peptide linker, and VL is the light chain variable domain, and/or optionally the scFv comprises an amino acid sequence selected from the group consisting of: SEQ ID Nos: 19-35 and 69-74.

In some embodiments, the chimeric protein is a chimeric antigen receptor (CAR), and the heterologous molecule or moiety comprises a polypeptide selected from the group consisting of: a transmembrane domain, one or more intracellular signaling domains, a hinge domain, a spacer region, one or more peptide linkers, and combinations thereof.

In some embodiments, the CAR is an inhibitory CAR comprising one or more intracellular inhibitory domains that inhibit an immune response and each of the one or more intracellular inhibitory domains comprises an enzymatic inhibitory domain or an intracellular inhibitory co-signaling domain.

In another aspect, provided herein is an engineered polynucleotide encoding the chimeric protein of any one of the above aspects or embodiments.

In another aspect, provided herein is an expression vector comprising the engineered polynucleotide of any one of the above aspects or embodiments.

In another aspect, provided herein is an isolated cell comprising the chimeric protein of any one of the above aspects or embodiments, the engineered polynucleotide of the above aspect or embodiments, or the expression vector of the above aspect or embodiments.

In another aspect, provided herein is a population of engineered cells expressing the chimeric protein of any one of the above aspects, the engineered polynucleotide of the aspect or embodiments, or the expression vector of the above aspect or embodiments.

In some embodiments, the cell or population of cells, further comprises one or more tumor-targeting chimeric receptors expressed on the cell surface, optionally wherein each of the one or more tumor-targeting chimeric receptors is a chimeric antigen receptor (CAR) or an engineered T cell receptor.

In some embodiments, the cell or population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

In another aspect, provided herein is a pharmaceutical composition comprising an effective amount of the cell or population of engineered cells of any one of the above aspects or embodiments and a pharmaceutically acceptable carrier, pharmaceutically acceptable excipient, or a combination thereof.

In another aspect, provided herein is a method of stimulating a cell-mediated immune response to a tumor cell in a subject, the method comprising administering to a subject having a tumor a therapeutically effective dose of the chimeric protein of any one of the above aspects or embodiments, the engineered polynucleotide of any one of the above aspects or embodiments, the expression vector of any one of the above aspects or embodiments, the cell or population of engineered cells of any one of the above aspects or embodiments, or the composition of the above aspects or embodiments.

In another aspect, provided herein is a method of treating a subject having a tumor, the method comprising administering a therapeutically effective dose of the chimeric protein of any one of the above aspects or embodiments, the engineered polynucleotide of any one of the above aspects or embodiments, the expression vector of any one of the above aspects or embodiments, the cell or population of engineered cells of any one of the above aspects or embodiments, or the composition of the above aspects or embodiments.

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, and wherein the VH comprises a heavy chain complementarity determining region 3 (CDR-H3) having the amino acid sequence of QGVRPFFDY (SEQ ID NO:4). In some aspects, the VL comprises a light chain complementarity determining region 1 (CDR-L1), a light chain complementarity determining region 2 (CDR-L2), and a light chain complementarity determining region 3 (CDR-L3), wherein the amino acid sequences of CDR-L1, CDR-L2, and CDR-L3 are contained within the VL region amino acid sequence of SEQ ID NO:9 or SEQ ID NO: 10. In some aspects, the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14).

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, wherein the VH comprises a heavy chain complementarity determining region 1 (CDR-H1), a heavy chain complementarity determining region 2 (CDR-H2), and a heavy chain complementarity determining region 3 (CDR-H3), and wherein the amino acid sequences of CDR-H1, CDR-H2, and CDR-H3 are contained within the VH region amino acid sequence of SEQ ID NO:1. In some aspects, the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14). In some aspects, the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14).

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, and wherein the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4). In some aspects, the VL comprises a light chain complementarity determining region 1 (CDR-L1), a light chain complementarity determining region 2 (CDR-L2), and a light chain complementarity determining region 3 (CDR-L3), wherein the amino acid sequences of CDR-L1, CDR-L2, and CDR-L3 are contained within the VL region amino acid sequence of SEQ ID NO:9 or SEQ ID NO: 10. In some aspects, the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14).

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, wherein the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region amino acid sequence of SEQ ID NO:9 or SEQ ID NO: 10. In some aspects, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region amino acid sequence of SEQ ID NO:1. In some aspects, the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4).

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, and wherein the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14). In some aspects, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region amino acid sequence of SEQ ID NO:1. In some aspects, the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4).

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, wherein the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4), and wherein the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of

(SEQ ID NO: 14)
QHHYVIPWT.

In some aspects, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1. In some aspects, the VH region comprises the amino acid sequence of SEQ ID NO:1.

In some aspects, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9. In some aspects, the VL region comprises the amino acid sequence of SEQ ID NO:9 or SEQ ID NO: 10.

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises a heavy chain variable (VH) region and a light chain variable (VL) region, and wherein the VH comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1. In some aspects, the VH region comprises the amino acid sequence of SEQ ID NO:1. In some aspects, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10. In some aspects, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9. In some aspects, the VL region comprises the amino acid sequence of SEQ ID NO:9. In some aspects, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:10. In some aspects, the VL region comprises the amino acid sequence of SEQ ID NO:10.

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain comprises an antibody or antigen-binding fragment thereof, wherein the antibody or antigen-binding fragment thereof comprises a heavy chain variable (VH) region and a light chain variable (VL) region, and wherein the VL comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10. In some aspects, the VL comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9. In some aspects, the VL region comprises the amino acid sequence of SEQ ID NO:10. In some aspects, the VL comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9. In some aspects, the VL region comprises the amino acid sequence of SEQ ID NO:10. In some aspects, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1. In some aspects, the VH region comprises the amino acid sequence of SEQ ID NO:1.

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain competes with a reference antibody or antigen-binding fragment thereof for binding to VSIG2, wherein the reference antibody or antigen-binding fragment thereof comprises a heavy chain variable (VH) region and a light chain variable (VL) region, wherein the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4), and wherein the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14). In some aspects, the VH region of the reference antibody or antigen-binding fragment thereof comprises the amino acid sequence of SEQ ID NO:1. In some aspects, the VL region of the reference antibody or antigen-binding fragment thereof comprises the amino acid sequence of SEQ ID NO:9 or SEQ ID NO: 10.

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain binds essentially the same VSIG2 epitope as a reference antibody or antigen-binding fragment thereof, wherein the reference antibody or antigen-binding fragment thereof comprises a heavy chain variable (VH) region and a light chain variable (VL) region, wherein the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4), and wherein the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14). In some aspects, the VH region of the reference antibody or antigen-binding fragment thereof comprises the amino acid sequence of SEQ ID NO:1. In some aspects, the VL region of the reference antibody or antigen-binding fragment thereof comprises the amino acid sequence of SEQ ID NO:9 or SEQ ID NO: 10.

In some aspects, the present disclosure provides a chimeric protein comprising an antigen-binding domain specific for V-Set And Immunoglobulin Domain Containing 2 (VSIG2) and a heterologous molecule or moiety, wherein the antigen-binding domain binds an epitope of human VSIG2 that is the same as the VSIG2 epitope bound by a reference antibody or antigen-binding fragment thereof, wherein the reference antibody or antigen-binding fragment thereof comprises a heavy chain variable (VH) region and a light chain variable (VL) region, wherein the VH comprises: a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4), and wherein the VL comprises: a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14). In some aspects, the VH region of the reference antibody or antigen-binding fragment thereof comprises the amino acid sequence of SEQ ID NO:1. In some aspects, the VL region of the reference antibody or antigen-binding fragment thereof comprises the amino acid sequence of SEQ ID NO:9 or SEQ ID NO: 10.

In some aspects, the antigen-binding domain of the chimeric protein comprises a F(ab) fragment, a F(ab′) fragment, or a single chain variable fragment (scFv). In some aspects, the antigen-binding domain comprises a single chain variable fragment (scFv). In some aspects, the VH and VL of the scFv are separated by a peptide linker. In some aspects, the antigen-binding domain comprises the structure VH-L-VL or VL-L-VH, wherein VH is the heavy chain variable domain, L is the peptide linker, and VL is the light chain variable domain. In some aspects, the peptide linker comprises an amino acid sequence selected from the group consisting of: SEQ ID Nos:19-35. In some aspects, the scFv comprises an amino acid sequence selected from the group consisting of SEQ ID NOs: 70-75.

In some aspects, the chimeric protein is an antibody-drug conjugate, and the heterologous molecule or moiety comprises a therapeutic agent.

In some aspects, the chimeric protein is a chimeric antigen receptor (CAR), and wherein the heterologous molecule or moiety comprises a polypeptide selected from the group consisting of: a transmembrane domain, one or more intracellular signaling domains, a hinge domain, a spacer region, one or more peptide linkers, and combinations thereof. In some aspects, the CAR comprises a transmembrane domain. In some aspects, the CAR comprises one or more intracellular signaling domains. In some aspects, the CAR is an activating CAR comprising one or more intracellular signaling domains that stimulate an immune response. In some aspects, the CAR is an inhibitory CAR comprising one or more intracellular inhibitory domains that inhibit an immune response. In some aspects, the intracellular inhibitory domain comprises an enzymatic inhibitory domain. In some aspects, the intracellular inhibitory domain comprises an intracellular inhibitory co-signaling domain. In some aspects, the CAR comprises a spacer region between the antigen-binding domain and the transmembrane domain. In some aspects, the spacer region has an amino acid sequence selected from the group consisting of SEQ ID NOs:39-51.

In some aspects, the present disclosure provides a composition comprising a chimeric protein as described herein and a pharmaceutically acceptable carrier, pharmaceutically acceptable excipient, or a combination thereof.

In some aspects, the present disclosure provides an engineered polynucleotide encoding a chimeric protein as described herein.

In some aspects, the present disclosure provides an expression vector comprising an engineered polynucleotide as described herein.

In some aspects, the present disclosure provides a composition comprising an engineered polynucleotide as described herein or an expression vector as described herein, and a pharmaceutically acceptable carrier, pharmaceutically acceptable excipient, or a combination thereof.

In some aspects, the present disclosure provides a method of making an engineered cell, comprising transducing an isolated cell with an engineered polynucleotide as described herein or an expression vector as described herein. In some aspects, the present disclosure provides an engineered cell produced by the above method.

In some aspects, the present disclosure provides an isolated cell comprising an engineered polynucleotide as described herein, an expression vector as described herein, or a composition comprising a engineered polynucleotide or expression vector as described herein and a pharmaceutically acceptable carrier, pharmaceutically acceptable excipient, or a combination thereof.

In some aspects, the present disclosure provides a population of engineered cells expressing an engineered polynucleotide as described herein or an expression vector as described herein.

In some aspects, the present disclosure provides an isolated cell comprising a chimeric protein as described herein. In some aspects, the present disclosure provides a population of engineered cells expressing a chimeric protein as described herein. In some aspects, the chimeric protein is recombinantly expressed in the isolated cell or the population of cells. In some aspects, the isolated cell or the population of cells further comprises one or more tumor-targeting chimeric receptors expressed on the cell surface. In some aspects, each of the one or more tumor-targeting chimeric receptors is a chimeric antigen receptors (CAR) or an engineered T cell receptor. In some aspects, the cell or population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some aspects, the cell or population of cells is/are autologous. In some aspects, the cell or population of cells is/are allogenic.

In some aspects, the present disclosure provides a pharmaceutical composition comprising an effective amount of the cell or population of engineered cells as described herein and a pharmaceutically acceptable carrier, pharmaceutically acceptable excipient, or a combination thereof.

In some aspects, the present disclosure provides a pharmaceutical composition comprising an effective amount of genetically modified cells expressing a chimeric protein as described herein and a pharmaceutically acceptable carrier, pharmaceutically acceptable excipient, or a combination thereof. In some aspects, the pharmaceutical composition is for treating and/or preventing a tumor.

In some aspects, the present disclosure provides a method for treating a subject in need thereof, the method comprising administering a therapeutically effective dose of a composition or pharmaceutical composition comprising a chimeric protein, polynucleotide, an expression vector as described herein, cell or population of cells as described herein.

In some aspects, the present disclosure provides a method of stimulating a cell-mediated immune response to a tumor cell in a subject, the method comprising administering to a subject having a tumor a therapeutically effective dose of a composition or pharmaceutical composition comprising a chimeric protein, polynucleotide, an expression vector as described herein, cell or population of cells as described herein.

In some aspects, the present disclosure provides a method of treating a subject having a tumor, the method comprising administering a therapeutically effective dose of a composition or pharmaceutical composition comprising a chimeric protein, polynucleotide, an expression vector as described herein, cell or population of cells as described herein.

In some aspects, the present disclosure provides a kit for treating and/or preventing a tumor, comprising a chimeric protein as described herein. In some aspects, the kit further comprises written instructions for using the chimeric protein for producing one or more antigen-specific cells for treating and/or preventing a tumor in a subject.

In some aspects, the present disclosure provides a kit for treating and/or preventing a tumor, comprising a cell or population of cells as described herein. In some aspects, the kit further comprises written instructions for using the cell for treating and/or preventing a tumor in a subject.

In some aspects, the present disclosure provides a kit for treating and/or preventing a tumor, comprising an isolated polynucleotide as described herein. In some aspects, the kit further comprises written instructions for using the polynucleotide for producing one or more antigen-specific cells for treating and/or preventing a tumor in a subject.

In some aspects, the present disclosure provides a kit for treating and/or preventing a tumor, comprising a vector as described herein. In some aspects, the kit further comprises written instructions for using the vector for producing one or more antigen-specific cells for treating and/or preventing a tumor in a subject.

In some aspects, the present disclosure provides a kit for treating and/or preventing a tumor, comprising a composition as described herein. In some aspects, the kit further comprises written instructions for using the composition for treating and/or preventing a tumor in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present disclosure will become better understood with regard to the following description, and accompanying drawings.

FIG. 1. Sequencing results for the light-chain variable regions of an anti-VSIG2 antibody (Ab) using Chothia naming scheme (SEQ ID NO: 103).

FIG. 2. Sequencing results for the heavy-chain variable regions of an anti-VSIG2 antibody(Ab) using Chothia naming scheme (SEQ ID NO: 104).

FIG. 3. Expression of Various anti-VSIG2 inhibitory CARs and an anti-CEA activating CAR from aCAR/iCAR transduced NK cells.

FIG. 4. Killing of CEA-positive target cells, either expressing VSIG2 or lacking VSIG2 expression, by NK cells expressing an anti-VSIG2 inhibitory CAR and an anti-CEA activating CAR.

FIG. 5. Killing of VSIG2-positive target cells by NK cells using anti-VSIG2 activating CAR. FIG. 5 discloses “(G4S)1” as SEQ ID NO: 29.

FIG. 6. Expression of various anti-VSIG2 activating CARs on transduced NK cells. FIG. 6 discloses “(G4S)3” as SEQ ID NO: 31 and “(G4S)1” as SEQ ID NO: 29.

FIG. 7A. Killing of target cells Ls174t by NK cells expressing anti-VSIG2 activating CAR.

FIG. 7B. Killing of target cells DLD1 by NK cells expressing anti-VSIG2 activating CAR.

FIG. 7C. Killing of target cells Ls174t by NK cells expressing anti-VSIG2 activating CAR.

FIG. 7D. Killing of target cells DLD1 by NK cells expressing anti-VSIG2 activating CAR.

FIG. 8. Killing of FLT3-positive target cells by NK cells expressing FLT3-activating CAR and various anti-VSIG2 inhibitory CARs. FIG. 8 discloses “(G4S)3” as SEQ ID NO: 31.

FIG. 9. Quantification of TNFα production in FLT3 activating CAR/VSIG2 inhibitory CAR NK cell/target cell co-culture. FIG. 9 discloses “(G4S)3” as SEQ ID NO: 31.

FIG. 10A. Expression of various anti-VSIG2 inhibitory CARs and anti-CEA activating CARs on transduced NK cells.

FIG. 10B. Expression of various anti-VSIG2 inhibitory CARs and anti-CEA activating CARs on control NK cells.

FIG. 11. Killing of CEA-positive target cells by NK cells expressing CEA-activating CAR and various anti-VSIG2 inhibitory CARs.

### DETAILED DESCRIPTION

The practice of the present disclosure will employ, unless otherwise indicated, conventional methods of molecular biology, chemistry, biochemistry, virology, and immunology, within the skill of the art. Such techniques are explained fully in the literature. See, e.g., Hepatitis C Viruses: Genomes and Molecular Biology (S. L. Tan ed., Taylor & Francis, 2006); Fundamental Virology, 3^{rd} Edition, vol. I & II (B. N. Fields and D. M. Knipe, eds.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

As used herein, the term “stimulating a cell-mediated immune response” or “stimulating an immune response” refers to generating a signal that results in an immune response by one or more cell types or cell populations. Immunostimulatory activity may include pro-inflammatory activity. In various embodiments, the immune response occurs after immune cell (e.g., T-cell or NK cell) activation or concomitantly mediated through receptors including, but not limited to, CD28, CD137 (4-1BB), OX40, CD40 and ICOS, and their corresponding ligands, including B7-1, B7-2, OX-40L, and 4-1BBL. Such polypeptides may be present in the tumor microenvironment and can activate immune responses to neoplastic cells. In various embodiments, promoting, stimulating, or otherwise agonizing pro-inflammatory polypeptides and/or their ligands may enhance the immune response of an immunoresponsive cell. Without being bound to a particular theory, receiving multiple stimulatory signals (e.g., co-stimulation) is important to mount a robust and long-term cell-mediated immune response, such as a T cell mediated immune response where T cells can become inhibited and unresponsive to antigen (also referred to as “T cell anergy”) in the absence of co-stimulatory signals. While the effects of the variety of co-stimulatory signals, particularly in combination with one another, can vary and remain only partially understood, co-stimulation generally results in increasing gene expression in order to generate long-lived, proliferative, and apoptotic resistant cells, such as T cells or NK cells, that robustly respond to antigen, for example in meditating complete and/or sustained eradication of targets cells expressing a cognate antigen.

As used herein, the term “chimeric antigen receptor” or alternatively a “CAR” refers to a recombinant polypeptide construct comprising at least an extracellular antigen-binding domain, a transmembrane domain and a cytoplasmic signaling domain (also referred to herein as “an intracellular signaling domain”) comprising a functional signaling domain.

As used herein, the term “activating CAR” or “aCAR” refers to CAR constructs/architectures capable of inducing signal transduction or changes in protein expression in the activating CAR-expressing cell that initiate, activate, stimulate, or increase an immune response upon binding to a cognate aCAR ligand.

As used herein, the term “inhibitory CAR” or “iCAR” refers to CAR constructs/architectures capable of inducing signal transduction or changes in protein expression in the inhibitory CAR-expressing cell that prevent, attenuate, inhibit, reduce, decrease, inhibit, or suppress an immune response upon binding to a cognate iCAR ligand, such as reduced activation of immunoresponsive cells receiving or having received one or more stimulatory signals, including co-stimulatory signals.

As used herein, the term “intracellular signaling domain” refers to the functional portion of a protein which acts by transmitting information within the cell to regulate cellular activity via defined signaling pathways by generating second messengers or functioning as effectors by responding to such messengers.

As used herein, the term “extracellular antigen-binding domain” or “antigen-binding domain” (ABD) refers to a polypeptide sequence or polypeptide complex that specifically recognizes or binds to a given antigen or epitope, such as the polypeptide sequence or polypeptide complex portion of the chimeric proteins described herein that provide the VSIG2-specific binding. An ABD (or antibody, antigen-binding fragment, and/or the chimeric protein including the same) is said to “recognize” the epitope (or more generally, the antigen) to which the ABD specifically binds, and the epitope is said to be the “recognition specificity” or “binding specificity” of the ABD. The ABD is said to bind to its specific antigen or epitope with a particular affinity. As described herein, “affinity” refers to the strength of interaction of non-covalent intermolecular forces between one molecule and another. The affinity, i.e., the strength of the interaction, can be expressed as a dissociation equilibrium constant (KD), wherein a lower KD value refers to a stronger interaction between molecules. KD values of antibody constructs are measured by methods well known in the art including, but not limited to, bio-layer interferometry (e.g. Octet/FORTEBIO®), surface plasmon resonance (SPR) technology (e.g. Biacore®), and cell binding assays (e.g., Flow-cytometry). Specific binding, as assessed by affinity, can refer to a binding molecule with an affinity between an ABD and its cognate antigen or epitope in which the KD value is below 10⁻⁶M, 10⁻⁷M, 10⁻⁸M, 10⁻⁹M, or 10⁻¹⁰M. Specific binding can also include recognition and binding of a biological molecule of interest (e.g., a polypeptide) while not specifically recognizing and binding other molecules in a sample, for example, a biological sample, which naturally includes a polypeptide of the present disclosure. In certain embodiments, specific binding refers to binding between an ABD, antibody, or antigen-binding fragment to an epitope or antigen or antigenic determinant in such a manner that binding can be displaced or competed with a second preparation of identical or similar epitope, antigen or antigenic determinant.

An ABD can be an antibody. The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

An ABD can be an antigen-binding fragment of an antibody. As used herein, the term “antigen-binding fragment” refers to at least one portion of an intact antibody, or recombinant variants thereof, that is sufficient to confer recognition and specific binding of the antigen-binding fragment to a target, such as an antigen or epitope. Examples of antigen-binding fragments include, but are not limited to, Fab, Fab′, F(ab′)₂, Fv, scFv, linear antibodies, single domain antibodies such as sdAb (either VL or VH), camelid V_{H}H domains, and multi-specific antibodies formed from antigen-binding fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1 136, 2005). Antigen binding fragments can also be grafted into scaffolds based on polypeptides such as a fibronectin type III (Fn3)(see U.S. Pat. No. 6,703,199, which describes fibronectin polypeptide minibodies).

The number of ABDs in a binding molecule, such as the chimeric proteins described herein, defines the “valency” of the binding molecule. A binding molecule having a single ABD is “monovalent”. A binding molecule having a plurality of ABDs is said to be “multivalent”. A multivalent binding molecule having two ABDs is “bivalent.” A multivalent binding molecule having three ABDs is “trivalent.” A multivalent binding molecule having four ABDs is “tetravalent.” In various multivalent embodiments, all of the plurality of ABDs have the same recognition specificity and can be referred to as a “monospecific multivalent” binding molecule. In other multivalent embodiments, at least two of the plurality of ABDs have different recognition specificities. Such binding molecules are multivalent and “multispecific.” In multivalent embodiments in which the ABDs collectively have two recognition specificities, the binding molecule is “bispecific.” In multivalent embodiments in which the ABDs collectively have three recognition specificities, the binding molecule is “trispecific.” In multivalent embodiments in which the ABDs collectively have a plurality of recognition specificities for different epitopes present on the same antigen, the binding molecule is “multiparatopic.” Multivalent embodiments in which the ABDs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABDs. Avidity can be measured by the same methods as those used to determine affinity, as described above. In certain embodiments, the avidity of a binding molecule for a specific target is such that the interaction is a specific binding interaction, wherein the avidity between two molecules has a KD value below 10⁻⁶M, 10⁻⁷M, 10⁻⁸M, 10⁻⁹M, or 10⁻¹⁰M. In certain embodiments, the avidity of a binding molecule for a specific target has a KD value such that the interaction is a specific binding interaction, wherein the one or more affinities of individual ABDs do not have has a KD value that qualifies as specifically binding their respective antigens or epitopes on their own. In certain embodiments, the avidity is the cumulative strength of interaction provided by the affinities of multiple ABDs for separate antigens on a shared specific target or complex, such as separate antigens found on an individual cell. In certain embodiments, the avidity is the cumulative strength of interaction provided by the affinities of multiple ABDs for separate epitopes on a shared individual antigen.

As used herein, the term “single-chain variable fragment” or “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked via a short flexible polypeptide linker, capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, “variable region” refers to a variable sequence that arises from a recombination event, for example, following V, J, and/or D segment recombination in an immunoglobulin gene in a B cell or T cell receptor (TCR) gene in a T cell. In immunoglobulin genes, variable regions are typically defined from the antibody chain from which they are derived, e.g., VH refers to the variable region of an antibody heavy chain and VL refers to the variable region of an antibody light chain. A select VH and select VL can associate together to form an antigen-binding domain that confers antigen specificity and binding affinity.

The term “complementarity determining region” or “CDR,” as used herein, refers to the sequences within antibody variable regions VH and VL which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise ammo acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al, (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and ammo acid residues 24-34 (LCDR1), 50-56 (LCDR2), and 89-97 (LCDR3) in a VL, e.g., a mammalian VL, e.g., a human VL. In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences.

The term “framework region” or “FR,” as used herein, refers to the generally conserved sequences within antibody variable regions VH and VL that act as a scaffold for interspersed CDRs, typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In specific embodiments, the FRs are human sequences. In various embodiments, the FRs are naturally occurring sequences. In various embodiments, the FRs are synthesized sequences including, but not limited, rationally designed sequences.

As used herein, the term “antibody heavy chain” refers to the larger of the two types of polypeptide chains present in antibody molecules in their naturally occurring conformations, and which normally determines the class to which the antibody belongs.

As used herein, the term “antibody light chain” refers to the smaller of the two types of polypeptide chains present in antibody molecules in their naturally occurring conformations. Kappa (κ) and lambda (λ) light chains refer to the two major antibody light chain isotypes.

As used herein, the term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

As used herein, the term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen.

As used herein, the term “anti-tumor effect” or “anti-tumor activity” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place, such as in a prophylactic therapy or treatment.

As used herein, the term “autologous” refers to any material derived from the same subject to whom it is later to be re-introduced into the subject.

As used herein, the term “allogeneic” refers to any material derived from a different animal of the same species as the subject to whom the material is introduced. Two or more subjects are said to be allogeneic to one another when the genes at one or more loci are not identical. In some embodiments, allogeneic material from individuals of the same species may be sufficiently genetically distinct, e.g., at particular genes such as MHC alleles, to interact antigenically. In some embodiments, allogeneic material from individuals of the same species may be sufficiently genetically similar, e.g., at particular genes such as MHC alleles, to not interact antigenically.

Isolated polynucleotide molecules of the present disclosure include any polynucleotide molecule or nucleic acid sequence that encodes a polypeptide of the present disclosure, or fragment thereof. Such polynucleotide molecules need not be 100% homologous or identical with an endogenous nucleic acid sequence, but will typically exhibit substantial identity. Nucleic acid sequences having “substantial identity” or “substantial homology” to an endogenous sequence are typically capable of hybridizing with at least one strand of a double-stranded polynucleotide molecule. As used herein, “hybridize” refers to pairing to form a double-stranded molecule between complementary polynucleotide sequences (e.g., a gene described herein), or portions thereof, under various conditions of stringency. For example, stringent salt concentration may be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency may be accomplished by combining these various conditions as needed.

By “substantially identical” or “substantially homologous” is meant a polypeptide or polynucleotide molecule exhibiting at least about 50% homologous or identical to a reference amino acid sequence (for example, any one of the amino acid sequences described herein) or nucleic acid sequence (for example, any one of the nucleic acid sequences described herein). Preferably, such a sequence is at least about 60%, about 80%, about 85%, about 90%, about 95%, about 99%, or about 100% homologous or identical at the amino acid level or nucleic acid level to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e−3 and e−100 indicating a closely related sequence.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some versions contain an intron(s).

As used herein, the term “ligand” refers to a molecule that binds to a receptor. In particular, the ligand binds a receptor on another cell, allowing for cell-to-cell recognition and/or interaction.

The terms “effective amount” and “therapeutically effective amount” are used interchangeably herein, and refer to an amount of a compound, formulation, material, or composition, as described herein effective to achieve a particular biological result. In some embodiments, an “effective amount” or a “therapeutically effective amount” is an amount sufficient to arrest, ameliorate, or inhibit the continued proliferation, growth, or metastasis of a disease or disorder of interest, e.g., a solid tumor.

As used herein, the term “immunoresponsive cell” refers to a cell that functions in an immune response (e.g., an immune effector response) or a progenitor, or progeny thereof. Examples of immune effector cells include, without limitation, alpha/beta T cells, gamma/delta T cells, B cells, natural killer (NK) cells, natural killer T (NKT) cells, mast cells, and myeloid-derived phagocytes.

As used herein, the term “immune effector response” or “immune effector function” refers to a function or response, e.g., of an immunoresponsive cell, that enhances or promotes an immune attack of a target cell. For example, an immune effector function or response may refer to a property of a T cell or NK cell that promotes killing or the inhibition of growth or proliferation, of a target cell. In the case of a T cell, primary stimulation and co-stimulation are examples of immune effector function or response.

As used herein, the term “flexible polypeptide linker” or “linker” refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Gly-Ser)ₙ (SEQ ID NO: 106) or (Gly-Gly-Gly-Ser)ₙ (SEQ ID NO: 107), where n is a positive integer equal to or greater than 1. For example, n=1, n=2, n=3, n=4, n=5, n=6, n=7, n=8, n=9, or n=10. In some embodiments, the flexible polypeptide linkers include, but are not limited to, Gly₄Ser [SEQ ID NO: 29] or (Gly₄Ser)₃ [SEQ ID NO: 31]. In other embodiments, the linkers include multiple repeats of (Gly₂Ser) (SEQ ID NO: 19), (GlySer) or (Gly₃Ser) [SEQ ID NO: 24]. In some embodiments, the flexible polypeptide linkers include a Whitlow linker (e.g., GSTSGSGKPGSGEGSTKG [SEQ ID NO:34]). In some embodiments, the flexible polypeptide linkers include an (EAAAK)₃ [SEQ ID NO:105] linker. Also included within the scope of the present disclosure are linkers described, for example, in WO2012/138475.

As used herein, the terms “treat”, “treatment” and “treating” refer to the reduction or amelioration of the progression, severity and/or duration of a proliferative disorder (e.g., cancer), or the amelioration of one or more symptoms (preferably, one or more discernible symptoms) of a proliferative disorder resulting from the administration of one or more therapies (e.g., one or more therapeutic agents such as a CAR of the present disclosure). In some embodiments, reduction or amelioration refers to the amelioration of at least one measurable physical parameter of a proliferative disorder, such as growth of a tumor, not necessarily discernible by the patient. In other embodiments, the terms “treat”, “treatment”, and “treating” refer to the inhibition of the progression of a proliferative disorder, either physically by, e.g., stabilization of a discernible symptom, physiologically by, e.g., stabilization of a physical parameter, or both. In some embodiments, reduction or amelioration include reduction or stabilization of tumor size or cancerous cell count.

As used herein, the term “subject” is intended to include living organisms in which an immune response can be elicited (e.g., mammals, human).

Other aspects of the present disclosure are described in the following sections and are within the ambit of the claimed invention.

### Other Interpretational Conventions

Ranges recited herein are understood to be shorthand for all of the values within the range, inclusive of the recited endpoints. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### VSIG2-Specific Chimeric Proteins and Antigen Binding Domains

The present disclosure provides chimeric proteins, and polynucleotides that encode such chimeric proteins, that bind to V-set and immunoglobulin domain-containing protein 2 (VSIG2). In some embodiments, VSIG2-specific chimeric proteins bind to human VSIG2 (e.g., Uniprot Q96IQ7, herein incorporated by reference for all purposes) or an epitope fragment thereof. VSIG2 can be expressed on epithelial cells. VSIG2 can be expressed on cells generally considered to be healthy, such as healthy epithelial cells. Examples of VSIG2-specific antibodies include OTI2D8 (also known as “2D8” and referred to herein as Ab) and OTI5A10 (also known as “5A10”).

The present disclosure provides chimeric proteins, and polynucleotides that encode such chimeric proteins, that include a VSIG2-specific antigen-binding domain having one or more of the amino acid sequences listed in Table A.

In some embodiments, the VSIG2-specific antigen-binding domain has a heavy chain variable (VH) region and a light chain variable (VL) region, and the VH comprises a heavy chain complementarity determining region 3 (CDR-H3) having the amino acid sequence of QGVRPFFDY (SEQ ID NO:4). In some embodiments, the VH further includes a heavy chain complementarity determining region 1 (CDR-H1), and a heavy chain complementarity determining region 2 (CDR-H2) having the amino acid sequences of CDR-H1 and CDR-H2 contained within the VH region amino acid sequence of SEQ ID NO:1. In some embodiments, the VSIG2-specific antigen-binding domain further includes a light chain complementarity determining region 1 (CDR-L1), a light chain complementarity determining region 2 (CDR-L2), and a light chain complementarity determining region 3 (CDR-L3), wherein the amino acid sequences of CDR-L1, CDR-L2, and CDR-L3 are contained within the VL region amino acid sequence of SEQ ID NO:9 or SEQ ID NO: 10. In some embodiments, the VSIG2-specific antigen-binding domain includes a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14).

In some embodiments, the VSIG2-specific antigen-binding domain has a heavy chain variable (VH) region and a light chain variable (VL) region in which the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO:1. In some embodiments, the VSIG2-specific antigen-binding domain further includes a light chain complementarity determining region 1 (CDR-L1), a light chain complementarity determining region 2 (CDR-L2), and a light chain complementarity determining region 3 (CDR-L3), wherein the amino acid sequences of CDR-L1, CDR-L2, and CDR-L3 are contained within the VL region amino acid sequence of SEQ ID NO:9 or SEQ ID NO: 10. In some embodiments, the VSIG2-specific antigen-binding domain includes a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14).

In some embodiments, the VSIG2-specific antigen-binding domain has a heavy chain variable (VH) region and a light chain variable (VL) region in which the VH includes a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4). In some embodiments, the VSIG2-specific antigen-binding domain further includes a light chain complementarity determining region 1 (CDR-L1), a light chain complementarity determining region 2 (CDR-L2), and a light chain complementarity determining region 3 (CDR-L3), wherein the amino acid sequences of CDR-L1, CDR-L2, and CDR-L3 are contained within the VL region amino acid sequence of SEQ ID NO:9 or SEQ ID NO: 10. In some embodiments, the VSIG2-specific antigen-binding domain includes a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14).

In some embodiments, the VSIG2-specific antigen-binding domain has a heavy chain variable (VH) region and a light chain variable (VL) region in which the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO:9 or SEQ ID NO: 10. In some embodiments, the VSIG2-specific antigen-binding domain further includes a heavy chain complementarity determining region (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region amino acid sequence of SEQ ID NO:1. In some embodiments, the VSIG2-specific antigen-binding domain includes a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of

(SEQ ID NO: 4)
QGVRPFFDY.

In some embodiments, the VSIG2-specific antigen-binding domain has a heavy chain variable (VH) region and a light chain variable (VL) region in which the VL includes a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of QHHYVIPWT (SEQ ID NO:14). In some embodiments, the VSIG2-specific antigen-binding domain further includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO:1. In some embodiments, the VSIG2-specific antigen-binding domain includes a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of

(SEQ ID NO: 4)
QGVRPFFDY.

In some embodiments, the VISIG2-specific antigen-binding domain has a heavy chain variable (VH) region and a light chain variable (VL) region in which; (1) the VH includes a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4), and (2) the VL includes a light chain complementarity determining region 1 (CDR-L1) having the amino acid sequence of RASENIYSYLA (SEQ ID NO:11) or RASENLYSYLA (SEQ ID NO:12), a light chain complementarity determining region 2 (CDR-L2) having the amino acid sequence of NAETLPE (SEQ ID NO:13), and a light chain complementarity determining region 3 (CDR-L3) having the amino acid sequence of

(SEQ ID NO: 14)
QHHYVIPWT.

In some embodiments, the VSIG2-specific antigen-binding domain has a VH region including the amino acid sequence of SEQ ID NO:1. In some embodiments, the VSIG2-specific antigen-binding domain has a VH region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1.

In some embodiments, the VSIG2-specific antigen-binding domain has a VL region including the amino acid sequence of SEQ ID NO:9 or SEQ ID NO: 10. In some embodiments, the VSIG2-specific antigen-binding domain has a VL region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10. In some embodiments, the VSIG2-specific antigen-binding domain has a (1) VH region including the amino acid sequence of SEQ ID NO:1, and (2) a VL region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10 or a VL region including the amino acid sequence of SEQ ID NO:9 or SEQ ID NO: 10.

In some embodiments, the VSIG2-specific antigen-binding domain has a (1) VH region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1, and (2) a VL region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10 or a VL region including the amino acid sequence of SEQ ID NO:9 or SEQ ID NO: 10.

In some embodiments, the VSIG2-specific antigen-binding domain has (1) a VL region including the amino acid sequence of SEQ ID NO:9 or SEQ ID NO: 10, and (2) a VH region including the amino acid sequence of SEQ ID NO:1 or a VH region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1. In some embodiments, the VSIG2-specific antigen-binding domain has a VL region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10 and (2) a VH region including the amino acid sequence of SEQ ID NO:1 or a VH region including an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1.

In some embodiments, the VSIG2-specific antigen-binding domain competes with a reference antibody or antigen-binding fragment thereof having a heavy chain variable (VH) region and a light chain variable (VL) region in which; (1) the VH includes a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO: 3), and a heavy chain complementarity determining region 3 (CDR-H3) having the amino acid sequence of QGVRPFFDY (SEQ ID NO: 4), and (2) the VL includes a light chain complementarity determining region 1 (CDR-L1) having the amino acid sequence of RASENIYSYLA (SEQ ID NO: 11) or RASENLYSYLA (SEQ ID NO:12), a light chain complementarity determining region 2 (CDR-L2) having the amino acid sequence of NAETLPE (SEQ ID NO: 13), and a light chain complementarity determining region 3 (CDR-L3) having the amino acid sequence of QHHYVIPWT (SEQ ID NO: 14).

In some embodiments, the VSIG2-specific antigen-binding domain binds the same or essentially the same epitope (e.g., a distinct human VSIG2 epitope) as a reference antibody or antigen-binding fragment thereof having a heavy chain variable (VH) region and a light chain variable (VL) region in which; (1) the VH includes a heavy chain complementarity determining region 1 (CDR-H1) having the amino acid sequence of GFTFSNS (SEQ ID NO:2), a heavy chain complementarity determining region 2 (CDR-H2) having the amino acid sequence of SDGGLY (SEQ ID NO:3), and a heavy chain complementarity determining region 3 (CDR-H3) having the amino acid sequence of QGVRPFFDY (SEQ ID NO:4), and (2) the VL includes a light chain complementarity determining region 1 (CDR-L1) having the amino acid sequence of RASENIYSYLA (SEQ ID NO: 11) or RASENLYSYLA (SEQ ID NO:12), a light chain complementarity determining region 2 (CDR-L2) having the amino acid sequence of NAETLPE (SEQ ID NO: 13), and a light chain complementarity determining region 3 (CDR-L3) having the amino acid sequence of QHHYVIPWT (SEQ ID NO: 14). In some embodiments, the VSIG2-specific antigen-binding domain binds the same or essentially the same epitope (e.g., a distinct human VSIG2 epitope) as a reference antibody or antigen-binding fragment thereof having a VH including the amino acid sequence of SEQ ID NO:1. In some embodiments, the VSIG2-specific antigen-binding domain binds the same or essentially the same epitope (e.g., a distinct human VSIG2 epitope) as a reference antibody or antigen-binding fragment thereof having a VL including the amino acid sequence of SEQ ID NO:9 or SEQ ID NO: 10.

The VSIG2-specific antigen-binding domain can be in any of the formats described herein, such as a Fab, Fab′, F(ab′)₂, Fv, scFv, linear antibody, single domain antibody such as sdAb (either VL or VH), camelid VHH, and multi-specific formats. In some embodiments, the VSIG2-specific antigen-binding domain is in a F(ab) format. In some embodiments, the VSIG2-specific antigen-binding domain is in a F(ab′) format.

In some embodiments, the VSIG2-specific antigen-binding domain is in a single chain variable fragment (scFv) format, including scFv formats having any of the peptide linkers described herein (e.g., see Table 1). In some embodiments, the VSIG2-specific antigen-binding domain has the structure VH-L-VL or VL-L-VH, where L is the peptide linker.

Chimeric Antigen Receptors (CARs)

Certain aspects of the present disclosure relate to chimeric receptors that have any one of the VSIG2-specific antigen-binding domain described herein and are capable of specifically binding to a VSIG2 protein, a VSIG2-derived antigen, or a VSIG2-derived epitope. In some embodiments, the chimeric receptor is a chimeric antigen receptor (CAR). In general, CARs are chimeric proteins that include an antigen-binding domain and polypeptide molecules that are heterologous to the antigen-binding domain, such as peptides heterologous to an antibody that an antigen-binding domain may be derived from. Polypeptide molecules that are heterologous to the antigen-binding domain can include, but are not limited to, a transmembrane domain, one or more intracellular signaling domains, a hinge domain, a spacer region, one or more peptide linkers, or combinations thereof.

In some embodiments, CARs are engineered receptors that graft or confer a specificity of interest (e.g., VSIG2) onto an immune effector cell. In certain embodiments, CARs can be used to graft the specificity of an antibody onto an immunoresponsive cell, such as a T cell or an NK cell. In some embodiments, CARs of the present disclosure comprise an extracellular antigen-binding domain (e.g., an scFv) fused to a transmembrane domain, fused to one or more intracellular signaling domains.

In some embodiments, the chimeric antigen receptor is an activating chimeric antigen receptor (aCAR and also generally referred to as CAR unless otherwise specified). In some embodiments, binding of the chimeric antigen receptor to its cognate ligand is sufficient to induce activation of the immunoresponsive cell. In some embodiments, binding of the chimeric antigen receptor to its cognate ligand is sufficient to induce stimulation of the immunoresponsive cell. In some embodiments, activation of an immunoresponsive cell results in killing of target cells. In some embodiments, activation of an immunoresponsive cell results in cytokine or chemokine expression and/or secretion by the immunoresponsive cell. In some embodiments, stimulation of an immunoresponsive cell results in cytokine or chemokine expression and/or secretion by the immunoresponsive cell. In some embodiments, stimulation of an immunoresponsive cell induces differentiation of the immunoresponsive cell. In some embodiments, stimulation of an immunoresponsive cell induces proliferation of the immunoresponsive cell. In some embodiments, activation and/or stimulation of the immunoresponsive cell can be combinations of the above responses.

A CAR of the present disclosure may be a first, second, or third generation CAR. “First generation” CARs comprise a single intracellular signaling domain, generally derived from a T cell receptor chain. “First generation” CARs generally have the intracellular signaling domain from the CD3-zeta (CD3C) chain, which is the primary transmitter of signals from endogenous TCRs. “First generation” CARs can provide de novo antigen recognition and cause activation of both CD4+ and CD8+ T cells through their CD3C chain signaling domain in a single fusion molecule, independent of HLA-mediated antigen presentation. “Second generation” CARs add a second intracellular signaling domain from one of various co-stimulatory molecules (e.g., CD28, 4-1BB, ICOS, OX40) to the cytoplasmic tail of the CAR to provide additional signals to the T cell. “Second generation” CARs provide both co-stimulation (e.g., CD28 or 4-1BB) and activation (CD3C). Preclinical studies have indicated that “Second Generation” CARs can improve the anti-tumor activity of immunoresponsive cell, such as a T cell. “Third generation” CARs have multiple intracellular co-stimulation signaling domains (e.g., CD28 and 4-IBB) and an intracellular activation signaling domain (CD3c).

In some embodiments, the chimeric antigen receptor is a chimeric inhibitory receptor (iCAR). In some embodiments, the one or more chimeric inhibitory receptors bind antigens that are expressed on a non-tumor cell derived from a tissue selected from brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor (e.g. a VSIG2-specific chimeric inhibitory receptor) may be used, for example, with one or more activating chimeric receptors (e.g., activating chimeric TCRs or CARs) expressed on a cell of the present disclosure (e.g., an immunoresponsive cell) as NOT-logic gates to control, modulate, or otherwise inhibit one or more activities of the one or more activating chimeric receptors. For instance, if a healthy cell expresses both an antigen that is recognized by a tumor-targeting chimeric receptor and an antigen that is recognized by an inhibitory chimeric receptor, an immunoresponsive cell expressing the tumor antigen may bind to the healthy cell. In such a case, the inhibitory chimeric antigen will also bind its cognate ligand on the healthy cell and the inhibitory function of the inhibitory chimeric receptor will reduce, decrease, prevent, or inhibit the activation of the immunoresponsive cell via the tumor-targeting chimeric receptor (“NOT-logic gating”). In some embodiments, a chimeric inhibitory receptor of the present disclosure may inhibit one or more activities of a cell of the present disclosure (e.g., an immunoresponsive cell). In some embodiments, an immunoresponsive cell may comprise one or more tumor-targeting chimeric receptors and one or more inhibitory chimeric receptors that targets an antigen that is not expressed, or generally considered to be expressed, on the tumor (e.g., VSIG2). Combinations of tumor-targeting chimeric receptors and inhibitory chimeric receptors in the same immunoresponsive cell may be used to reduce on-target off-tumor toxicity.

In some embodiments, the extracellular antigen-binding domain of a CAR of the present disclosure binds to one or more antigens (e.g., VSIG2) with a dissociation constant (K_{d}) of about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ M or less, about 2×10⁻⁹ M or less, or about 1×10⁻⁹ M or less. In some embodiments, the K_{d} ranges from about is about 2×10⁻⁷ M to about 1×10⁻⁹ M.

Binding of the extracellular antigen-binding domain of a CAR of the present disclosure can be determined by, for example, an enzyme-linked immunosorbent assay (ELISA), a radioimmunoassay (RIA), FACS analysis, a bioassay (e.g., growth inhibition), bio-layer interferometry (e.g. Octet/FORTEBIO®), surface plasmon resonance (SPR) technology (e.g. Biacore®), or a Western Blot assay. Each of these assays generally detect the presence of protein-antibody complexes of particular interest by employing a labeled reagent (e.g., an antibody or scFv) specific for the complex of interest. For example, the scFv can be radioactively labeled and used in an RIA assay. The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the extracellular antigen-binding domain of the CAR is labeled with a secondary antibody specific for the extracellular antigen-binding domain and wherein the secondary antibody is labeled (e.g., radioactively or with a fluorescent marker).

In some embodiments, CARs of the present disclosure comprise an extracellular antigen-binding domain that binds to VSIG2 (e.g., a VSIG2 protein, a VSIG2-derived antigen, or a VSIG2-derived epitope), a transmembrane domain, and one or more intracellular signaling domains. In some embodiments, the extracellular antigen-binding domain comprises an scFv. In some embodiments, the extracellular antigen-binding domain comprises a Fab fragment, which may be crosslinked. In certain embodiments, the extracellular binding domain is a F(ab)₂ fragment.

### Extracellular Antigen-Binding Domain

The extracellular antigen-binding domain of a CAR of the present disclosure specifically binds to VSIG2 (e.g., a VSIG2 protein, a VSIG2-derived antigen, or a VSIG2-derived epitope). In certain embodiments, the extracellular antigen-binding domain binds to VSIG2 expressed on an epithelial cell. In certain embodiments, the extracellular antigen-binding domain binds to VSIG2 expressed on cells generally considered to be healthy, such as healthy epithelial cells. In some embodiments, VSIG2 is human VSIG2.

Antigen-binding domains of the present disclosure can include any domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (VHH) of camelid derived nanobody, and to an alternative scaffold known in the art to function as antigen-binding domain, such as a recombinant fibronectin domain, a T cell receptor (TCR), a recombinant TCR with enhanced affinity, or a fragment thereof, e.g., single chain TCR, and the like. In some instances, it is beneficial for the antigen-binding domain to be derived from the same species in which the CAR will ultimately be used in.

In some embodiments, the extracellular antigen-binding domain comprises an antibody. In certain embodiments, the antibody is a human antibody. In certain embodiments, the antibody is a chimeric antibody. In some embodiments, the extracellular antigen-binding domain comprises an antigen-binding fragment of an antibody.

In some embodiments, the extracellular antigen-binding domain comprises a F(ab) fragment. In certain embodiments, the extracellular antigen-binding domain comprises a F(ab′) fragment.

In some embodiments, the extracellular antigen-binding domain comprises an scFv. In some embodiments, the extracellular antigen-binding domain comprises two single chain variable fragments (scFvs). In some embodiments, each of the two scFvs binds to a distinct epitope on the same antigen. In some embodiments, the extracellular antigen-binding domain comprises a first scFv and a second scFv. In some embodiments, the first scFv and the second scFv bind distinct epitopes on the same antigen. In certain embodiments, the scFv is a mammalian scFv. In certain embodiments, the scFv is a chimeric scFv. In certain embodiments, the scFv comprises a heavy chain variable domain (VH) and a light chain variable domain (VL).

In some embodiments, the scFv comprises the amino acid sequence of SEQ ID NO:69. In some embodiments, the scFv comprises the amino acid sequence of SEQ ID NO:70. In some embodiments, the scFv comprises the amino acid sequence of SEQ ID NO:71. In some embodiments, the scFv comprises the amino acid sequence of SEQ ID NO:72. In some embodiments, the scFv comprises the amino acid sequence of SEQ ID NO:73. In some embodiments, the scFv comprises the amino acid sequence of SEQ ID NO:74.

In some embodiments, a polynucleotide encoding an scFv of the present disclosure comprises the nucleic acid sequence of SEQ ID NO:75. In some embodiments, a polynucleotide encoding an scFv of the present disclosure comprises the nucleic acid sequence of SEQ ID NO: 76. In some embodiments, a polynucleotide encoding an scFv of the present disclosure comprises the nucleic acid sequence of SEQ ID NO:77. In some embodiments, a polynucleotide encoding an scFv of the present disclosure comprises the nucleic acid sequence of SEQ ID NO: 78 In some embodiments, a polynucleotide encoding an scFv of the present disclosure comprises the nucleic acid sequence of SEQ ID NO:79. In some embodiments, a polynucleotide encoding an scFv of the present disclosure comprises the nucleic acid sequence of SEQ ID NO:80. In some embodiments, a polynucleotide encoding an scFv of the present disclosure comprises the nucleic acid sequence of SEQ ID NO:81. In some embodiments, a polynucleotide encoding an scFv of the present disclosure comprises the nucleic acid sequence of SEQ ID NO:82. In some embodiments, a polynucleotide encoding an scFv of the present disclosure comprises the nucleic acid sequence of SEQ ID NO:83. In some embodiments, a polynucleotide encoding an scFv of the present disclosure comprises the nucleic acid sequence of SEQ ID NO:84. In certain embodiments, the VH and VL are separated by a peptide linker. In certain embodiments, the peptide linker comprises any of the amino acid sequences shown in Table 1. In certain embodiments, the scFv comprises the structure VH-L-VL or VL-L-VH, wherein VH is the heavy chain variable domain, L is the peptide linker, and VL is the light chain variable domain. In some embodiments, each of the one or more scFvs comprises the structure VH-L-VL or VL-L-VH, wherein VH is the heavy chain variable domain, L is the peptide linker, and VL is the light chain variable domain. When there are two or more scFv linked together, each scFv can be linked to the next scFv with a peptide linked. In some embodiments, each of the one or more scFvs is separated by a peptide linker.

TABLE 1
Peptide Linkers

| | | SEQ |
|---|---|---|
| | | ID |
| Linker | Amino Acid Sequence | NO: |
| (G₂S)₁ linker | GGS | 19 |
| (G₂S)₂ linker | GGSGGS | 20 |
| (G₂S)₃ linker | GGSGGSGGS | 21 |
| (G₂S)₄ linker | GGSGGSGGSGGS | 22 |
| (G₂S)₅ linker | GGSGGSGGSGGSGGS | 23 |
| (G₃S)₁ linker | GGGS | 24 |
| (G₃S)₂ linker | GGGSGGGS | 25 |
| (G₃S)₃ linker | GGGSGGGSGGGS | 26 |
| (G₃S)₄ linker | GGGSGGGSGGGSGGGS | 27 |
| (G₃S)₅ linker | GGGSGGGSGGGSGGGSGGGS | 28 |
| (G₄S)₁ linker | GGGGS | 29 |
| (G₄S)₂ linker | GGGGSGGGGS | 30 |
| (G₄S)₃ linker | GGGGSGGGGSGGGGS | 31 |
| (G₄S)₄ linker | GGGGSGGGGSGGGGSGGGGS | 32 |
| (G₄S)₅ linker | GGGGSGGGGSGGGGSGGGGSGGGGS | 33 |
| Whitlow linker | GSTSGSGKPGSGEGSTKG | 34 |
| (EAAAK)4 linker 2 | EAAAKEAAAKEAAAKEAAAK | 35 |

In some embodiments, the present disclosure provides a first CAR and a second CAR. The antigen binding domain of the first CAR and the antigen binding domain of the second CAR can be an appropriate antigen biding domain described herein or known in the art. For example, the first or second antigen binding domain can be one or more antibodies, antigen-binding fragments of an antibody, F(ab) fragments, F(ab′) fragments, single chain variable fragments (scFvs), or single-domain antibodies (sdAbs). In some embodiments, the antigen-binding domain of the first CAR and/or the second CAR comprises two single chain variable fragments (scFvs). In some embodiments, each of the two scFvs binds to a distinct epitope on the same antigen. In some embodiments, the antigen binding domain of the first CAR can be specific for VSIG2 and the antigen binding domain of the second CAR can be specific for a second distinct antigen, such as a cancer antigen (e.g., an antigen expressed on a tumor cell, such as a colorectal cancer cell).

In some embodiments, the extracellular antigen-binding domain comprises a single-domain antibody (sdAb). In certain embodiments, the sdAb is a humanized sdAb. In certain embodiments, the sdAb is a chimeric sdAb.

In some embodiments, a CAR of the present disclosure may comprise two or more antigen-binding domains, three or more antigen-binding domains, four or more antigen-binding domains, five or more antigen-binding domains, six or more antigen-binding domains, seven or more antigen-binding domains, eight or more antigen-binding domains, nine or more antigen-binding domains, or ten or more antigen-binding domains. In some embodiments, each of the two or more antigen-binding domains binds the same antigen. In some embodiments, each of the two or more antigen-binding domains binds a different epitope of the same antigen. In some embodiments, each of the two or more antigen-binding domains binds a different antigen.

In some embodiments, the CAR comprises two antigen-binding domains. In some embodiments, the two antigen-binding domains are attached to one another via a flexible linker. In some embodiments, each of the two-antigen-binding domains may be independently selected from an antibody, an antigen-binding fragment of an antibody, an scFv, a sdAb, a recombinant fibronectin domain, a T cell receptor (TCR), a recombinant TCR with enhanced affinity, and a single chain TCR. In some embodiments, the CAR comprising two antigen-binding domains is a bispecific CAR or a tandem CAR (tanCAR).

In certain embodiments, the bispecific CAR or tanCAR comprises an antigen-binding domain comprising a bispecific antibody or antibody fragment (e.g., scFv). In some embodiments, within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁ VH₁-VH₂—VL₂. In some embodiments, a linker is disposed between the two antibodies or antibody fragments (e.g., scFvs), for example, between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6 (SEQ ID NO: 101). In general, the linker between the two scFvs should be long enough to avoid mispairing between the domains of the two scFvs. In some embodiments, a linker is disposed between the VL and VH of the first scFv. In some embodiments, a linker is disposed between the VL and VH of the second scFv. In constructs that have multiple linkers, any two or more of the linkers may be the same or different. Accordingly, in some embodiments, a bispecific CAR or tanCAR comprises VLs, VHs, and may further comprise one or more linkers in an arrangement as described herein.

In some embodiments, a chimeric receptor of the present disclosure comprises a bivalent CAR. In some embodiments, the bivalent CAR is a VSIG2 bivalent CAR. In some embodiments, the bivalent VSIG2 CAR comprises one or more of the anti-VSIG2 sequences shown in Table A. In some embodiments, the ABDs of the bivalent VSIG2 CAR each comprises the same ABD.

In some embodiments, chimeric receptors comprise a bicistronic chimeric antigen receptor. In some embodiments, the bicistronic chimeric antigen receptor comprises a VSIG2 CAR. In some embodiments, the bicistronic VSIG2 CAR comprises one or more of the anti-VSIG2 sequences shown in Table A.

### Transmembrane Domain

In some embodiments, the transmembrane domain of a CAR of the present disclosure (e.g. the VSIG2-specific CARs described herein) comprises a hydrophobic alpha helix that spans at least a portion of a cell membrane. It has been shown that different transmembrane domains can result in different receptor stability. After antigen recognition, receptors cluster and a signal is transmitted to the cell. In some embodiments, the transmembrane domain of a CAR of the present disclosure can comprise the transmembrane domain of a CD8 polypeptide, a CD28 polypeptide, a CD3-zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a LIR-1 (LILRB1) polypeptide, or can be a synthetic peptide, or any combination thereof.

In some embodiments, the transmembrane domain is derived from a CD8 polypeptide. Any suitable CD8 polypeptide may be used. Exemplary CD8 polypeptides include, without limitation, NCBI Reference Nos. NP_001139345 and AAA92533.1. Examples of CD8 transmembrane domains include IYIWAPLAGTCGVLLLSLVIT (SEQ ID NO:36), IYIWAPLAGTCGVLLLSLVITLYCNHR (SEQ ID NO:37), and IYIWAPLAGTCGVLLLSLVITLYCNHRN (SEQ ID NO:38). In some embodiments, the transmembrane domain comprises the sequence IYIWAPLAGTCGVLLLSLVIT (SEQ ID NO:36). In some embodiments, the transmembrane domain comprises the sequence IYIWAPLAGTCGVLLLSLVITLYCNHR (SEQ ID NO:37). In some embodiments, the transmembrane domain comprises the sequence IYIWAPLAGTCGVLLLSLVITLYCNHRN (SEQ ID NO:38).

In some embodiments, the transmembrane domain is derived from a CD28 polypeptide. Any suitable CD28 polypeptide may be used. Exemplary CD28 polypeptides include, without limitation, NCBI Reference Nos. NP 006130.1 and NP 031668.3. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1. In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide. Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. Any suitable LIR-1 (LILRB1) polypeptide may be used. Exemplary LIR-1 (LILRB1) polypeptides include, without limitation, NCBI Reference Nos. NP_001075106.2 and NP_001075107.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_001139345, AAA92533.1, NP_006130.1, NP 031668.3, NP_932170.1, NP_001106862.1, NP 000607.1, NP_038516.1, NP_001552.2, NP_001070977.1, NP 003318.1, NP_035789.1, NP 036224, NP_059508, NP_005205.2, NP 033973.2, NP_005009, NP 032824, NP_002277.4, NP_032505.1, NP_057466.1, NP_061199.2, NP_861445.4, or NP_001032808.2, or fragments thereof. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_001139345, AAA92533.1, NP_006130.1, NP_031668.3, NP 932170.1, NP 001106862.1, NP_000607.1, NP 038516.1, NP_001552.2, NP 001070977.1, NP_003318.1, NP 035789.1, NP_036224, NP 059508, NP_005205.2, NP_033973.2, NP 005009, NP_032824, NP 002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Rα, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NG2C.

In some embodiments, the transmembrane domain is derived from the same protein as an intracellular domain of the CAR.

### Spacer Region

In some embodiments, a CAR of the present disclosure (e.g. the VSIG2-specific CARs described herein) can also comprise a spacer region that links the extracellular antigen-binding domain to the transmembrane domain. The spacer region may be flexible enough to allow the antigen-binding domain to orient in different directions to facilitate antigen recognition. In some embodiments, the spacer region may be a hinge from a human protein. For example, the hinge may be a human Ig (immunoglobulin) hinge, including without limitation an IgG4 hinge, an IgG2 hinge, a CD8a hinge, or an IgD hinge. In some embodiments, the spacer region may comprise an IgG4 hinge, an IgG2 hinge, an IgD hinge, a CD28 hinge, a KIR2DS2 hinge, an LNGFR hinge, or a PDGFR-beta extracellular linker. In some embodiments, the spacer region is localized between the antigen-binding domain and the transmembrane domain. In some embodiments, a spacer region may comprise any of the amino acid sequences listed in Table 2, or an amino acid sequence that is at least 90%, at least 91%, at least 92%, at least 93%, at least 94%, at least 95%, at least 96%, at least 97%, at least 98%, or at least 99% identical to any of the amino acid sequences listed in Table 2.

TABLE 2
Spacer Amino Acid Sequences

| Amino Acid Sequence | SEQ ID NO: | Description |
|---|---|---|
| TTTPAPRPPTPAPTIALQPLSLRPEACRPAAGGAVHTRGL | 39 | CD8 hinge |
| DFACD | | |
| AAAIEVMYPPPYLDNEKSNGTIIHVKGKHLCPSPLFPGPS | 40 | CD28 hinge |
| KP | | |
| ESKYGPPCPSCP | 41 | IgG4 minimal hinge |
| ESKYGPPAPSAP | 42 | IgG4 minimal hinge, no |
| | | disulfides |
| ESKYGPPCPPCP | 43 | IgG4 S228P minimal hinge, |
| | | enhanced disulfide formation |
| EPKSCDKTHTCP | 44 | IgG1 minimal hinge |
| AAAFVPVFLPAKPTTTPAPRPPTPAPTIASQPLSLRPEAC | 45 | Extended CD8a hinge |
| RPAAGGAVHTRGLDFACDIYIWAPLAGTCGVLLLSLVIT | | |
| LYCNHRN | | |
| ACPTGLYTHSGECCKACNLGEGVAQPCGANQTVCEPCL | 46 | LNGFR hinge |
| DSVTFSDVVSATEPCKPCTECVGLQSMSAPCVEADDAV | | |
| CRCAYGYYQDETTGRCEACRVCEAGSGLVFSCQDKQN | | |
| TVCEECPDGTYSDEADAEC | | |
| ACPTGLYTHSGECCKACNLGEGVAQPCGANQTVC | 47 | Truncated LNGFR hinge |
| | | (TNFR-Cys1) |
| AVGQDTQEVIVVPHSLPFKV | 48 | PDGFR-beta extracellular |
| | | linker |

In some embodiments, the spacer region comprises the sequence shown in SEQ ID NO:39. In some embodiments, the spacer region comprises the sequence shown in SEQ ID NO:40. In some embodiments, the spacer region comprises the sequence shown in SEQ ID NO:41. In some embodiments, the spacer region comprises the sequence shown in SEQ ID NO:42. In some embodiments, the spacer region comprises the sequence shown in SEQ ID NO:43. In some embodiments, the spacer region comprises the sequence shown in SEQ ID NO:44. In some embodiments, the spacer region comprises the sequence shown in SEQ ID NO:45. In some embodiments, the spacer region comprises the sequence shown in SEQ ID NO:46. In some embodiments, the spacer region comprises the sequence shown in SEQ ID NO:47. In some embodiments, the spacer region comprises the sequence shown in SEQ ID NO:48.

In some embodiments, the spacer region comprises the sequence TTTPAPRPPTPAPTIALQPLSLRPEACRPAAGGAVHTRGLDFACD (SEQ ID NO:49). In some embodiments, the spacer region comprises the sequence ALSNSIMYFSHFVPVFLPAKPTTTPAPRPPTPAPTIASQPLSLRPEACRPAAGGAVHTRGL DFACD (SEQ ID NO:50). In some embodiments, the spacer region comprises the sequence

(SEQ ID NO: 51)
FVPVFLPAKPTTTPAPRPPTPAPTIALQPLSLRPEACRPAAGGAVHTRG
LDFACD.

In some embodiments, polynucleotides encoding any of the spacer regions of the present disclosure may comprise any of the nucleic acid sequences listed in Table 3, or a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 3.

TABLE 3
Spacer Nucleic Acid Sequences

| Nucleic Acid Sequence | SEQ ID NO: | Description |
|---|---|---|
| ACCACAACTCCTGCTCCTAGACCTCCTACACCAGCTC | 52 | CD8 hinge |
| CTACAATCGCCCTGCAGCCACTGTCTCTGAGGCCAGA | | |
| AGCTTGTAGACCTGCTGCAGGCGGAGCCGTGCATACA | | |
| AGAGGACTGGATTTCGCCTGCGAC | | |
| GCAGCAGCTATCGAGGTGATGTATCCTCCGCCCTACC | 53 | CD28 hinge |
| TGGATAATGAAAAGAGTAATGGGACTATCATTCATGT | | |
| AAAAGGGAAGCATCTTTGTCCTTCTCCCCTTTTCCCCG | | |
| GTCCGTCTAAACCT | | |
| GAAAGCAAGTACGGTCCACCTTGCCCTAGCTGTCCG | 54 | IgG4 minimal hinge |
| GAATCCAAGTACGGCCCCCCAGCGCCTAGTGCCCCA | 55 | IgG4 minimal hinge, no |
| | | disulfides |
| GAATCTAAATATGGCCCGCCATGCCCGCCTTGCCCA | 56 | IgG4 S228P minimal hinge, |
| | | enhanced disulfide formation |
| GAACCGAAGTCTTGTGATAAAACTCATACGTGCCCG | 57 | IgG1 minimal hinge |
| GCTGCTGCTTTCGTACCCGTGTTCCTCCCTGCTAAGCC | 58 | Extended CD8a hinge |
| TACGACTACCCCCGCACCGAGACCACCCACGCCAGC | | |
| ACCCACGATTGCTAGCCAGCCCCTTAGTTTGCGACCA | | |
| GAAGCTTGTCGGCCTGCTGCTGGTGGCGCGGTACATA | | |
| CCCGCGGCCTTGATTTTGCTTGCGATATATATATCTGG | | |
| GCGCCTCTGGCCGGAACATGCGGGGTCCTCCTCCTTT | | |
| CTCTGGTTATTACTCTCTACTGTAATCACAGGAAT | | |
| GCCTGCCCGACCGGGCTCTACACTCATAGCGGGGAAT | 59 | LNGFR hinge |
| GTTGTAAGGCATGTAACTTGGGTGAGGGCGTCGCACA | | |
| GCCCTGCGGAGCTAACCAAACAGTGTGCGAACCCTG | | |
| CCTCGATAGTGTGACGTTCTCTGATGTTGTATCAGCTA | | |
| CAGAGCCTTGCAAACCATGTACTGAGTGCGTTGGACT | | |
| TCAGTCAATGAGCGCTCCATGTGTGGAGGCAGATGAT | | |
| GCGGTCTGTCGATGTGCTTACGGATACTACCAAGACG | | |
| AGACAACAGGGCGGTGCGAGGCCTGTAGAGTTTGTG | | |
| AGGCGGGCTCCGGGCTGGTGTTTTCATGTCAAGACAA | | |
| GCAAAATACGGTCTGTGAAGAGTGCCCTGATGGCACC | | |
| TACTCAGACGAAGCAGATGCAGAATGC | | |
| GCCTGCCCTACAGGACTCTACACGCATAGCGGTGAGT | 60 | Truncated LNGFR hinge |
| GTTGTAAAGCATGCAACCTCGGGGAAGGTGTAGCCC | | (TNFR-Cys1) |
| AGCCATGCGGGGCTAACCAAACCGTTTGC | | |
| GCTGTGGGCCAGGACACGCAGGAGGTCATCGTGGTG | 61 | PDGFR-beta extracellular |
| CCACACTCCTTGCCCTTTAAGGTG | | linker |

In some embodiments, a CAR of the present disclosure may further include a short oligopeptide or polypeptide linker that is between 2 amino acid residues and 10 amino acid residues in length, and that may form the linkage between the transmembrane domain and the cytoplasmic region of the CAR. A non-limiting example of a suitable linker is a glycine-serine doublet. In some embodiments, the linker comprises the ammo acid sequence of

(SEQ ID NO: 62)
GGCKJSGGCKJS.

### Intracellular Signaling Domains

In some embodiments, a CAR of the present disclosure (e.g. the VSIG2-specific CARs described herein) comprises one or more cytoplasmic domains or regions. The cytoplasmic domain or region of the CAR may include an intracellular signaling domain.

Examples of suitable intracellular signaling domains that may be used in CARs of the present disclosure include, without limitation, cytoplasmic sequences of the T cell receptor (TCR) and co-receptors that act in concert to modulate signal transduction following antigen receptor engagement, as well as any derivative or variant of these sequences and any recombinant sequence that has the same functional capability.

Without wishing to be bound by theory, it is believed that signals generated through the TCR alone are insufficient for full activation of the T cell and that a secondary and/or co-stimulatory signal is thus also typically required for full activation. Accordingly, T cell activation may be mediated by two distinct classes of cytoplasmic signaling sequences, those that initiate antigen-dependent primary activation through the TCR (primary intracellular signaling domains) and those that act in an antigen-independent manner to provide a secondary or co-stimulatory signal (secondary cytoplasmic domain, e.g., a co-stimulatory domain). In addition, T cell signaling and function (e.g., an activating signaling cascade) can be negatively regulated by inhibitory receptors present in a T cell through intracellular inhibitory co-signaling domains.

In some embodiments, the intracellular signaling domain of a CAR of the present disclosure can include an inhibitory intracellular signaling domains. In some embodiments, the inhibitory intracellular signaling domain includes one or more intracellular inhibitory co-signaling domains. In some embodiments, the one or more intracellular inhibitory co-signaling domains are linked to other domains (e.g., a transmembrane domain) through a peptide linker (e.g., see Table 1) or a spacer or hinge sequence (e.g., see Table 2). In some embodiments, when two or more intracellular inhibitory co-signaling domains are present, the two or more intracellular inhibitory co-signaling domains can be linked through a peptide linker (e.g., see Table 1) or a spacer or hinge sequence (e.g., see Table 2). In some embodiments, the intracellular inhibitory co-signaling domain is an inhibitory domain. In some embodiments, the one or more intracellular inhibitory co-signaling domains of a chimeric protein comprises one or more ITIM-containing protein, or fragment(s) thereof. ITIMs are conserved amino acid sequences found in cytoplasmic tails of many inhibitory immune receptors. In some embodiments, the one or more ITIM-containing protein, or fragments thereof, is selected from PD-1, CTLA4, TIGIT, BTLA, LAIR1, KIR2DL1, KIR3DL1, LIR1, SIGLEC2, and SIRPalpha (SIRPa). In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAG3, HAVR, GITR, and PD-L1. The inhibitory intracellular signaling domain can further include an enzymatic inhibitory domains. In some embodiments, the enzymatic inhibitory domain comprises an enzyme catalytic domain. In some embodiments, the enzyme catalytic domain is derived from an enzyme including, but not limited to, CSK, SUP-1, PTEN, CD45, CD148, PTP-MEG1, PTP-PEST, c-CBL, CBL-b, PTPN22, LAR, PTPH1, SHIP-1, or RasGAP. Examples of enzymatic regulation of signaling is described in more detail in Pavel Otihal et al. (Biochim Biophys Acta. 2011 February; 1813(2):367-76), Kosugi A., et al. (Involvement of SHP-1 tyrosine phosphatase in TCR-mediated signaling pathways in lipid rafts, Immunity, 2001 June; 14(6): 669-80), and Stanford, et al. (Regulation of TCR signaling by tyrosine phosphatases: from immune homeostasis to autoimmunity, Immunology, 2012 September; 137(1): 1-19), each of which is incorporated herein by reference for all purposes.

In some embodiments, the intracellular signaling domain of a CAR of the present disclosure can comprise a primary signaling domain regulates primary activation of the TCR complex either in a stimulatory way or in an inhibitory way. Primary intracellular signaling domains that act in a stimulatory manner may contain signaling motifs which are known as immunoreceptor tyrosine-based activation motifs (ITAMs). Examples of suitable ITAM-containing primary intracellular signaling domains that that may be used in the CARs of the present disclosure include, without limitation, those of CD3-zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d.

In some embodiments, a CAR of the present disclosure (e.g. the VSIG2-specific CARs described herein) comprises an intracellular signaling domain, e.g., a primary signaling domain of CD3-zeta polypeptide. A CD3-zeta polypeptide of the present disclosure may have an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_932170 or NP_001106864.2, or fragments thereof. In some embodiments, the CD3-zeta polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_932170 or NP_001106864.2 that is at least 20, at least 30, at least 40, at least 50, at least 60, at least 70, at least 80, at least 90, at least 100, at least 110, at least 120, at least 130, at least 140, at least 150, or at least 160, at least 170, or at least 180 amino acids in length.

In other embodiments, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In one embodiment, a primary signaling domain comprises one, two, three, four or more ITAM motifs.

In some embodiments, the intracellular signaling domain of a CAR of the present disclosure can comprise the CD3-zeta signaling domain by itself or it can be combined with any other desired intracellular signaling domain(s) useful in the context of a CAR of the present disclosure. For example, the intracellular signaling domain of the CAR can comprise a CD3-zeta chain portion and a costimulatory signaling domain. The costimulatory signaling domain may refer to a portion of the CAR comprising the intracellular domain of a costimulatory molecule. A costimulatory molecule of the present disclosure is a cell surface molecule other than an antigen receptor or its ligands that may be required for an efficient response of lymphocytes to an antigen. Examples of suitable costimulatory molecules include, without limitation, CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and the like.

In some embodiments, the intracellular signaling sequences within the cytoplasmic portion of a CAR of the present disclosure may be linked to each other in a random or specified order. In some embodiments, a short oligopeptide or polypeptide linker, for example, between 2 amino acids and 10 amino acids (e.g., 2 amino acids, 3 amino acids, 4 amino acids, 5 amino acids, 6 amino acids, 7 amino acids, 8 amino acids, 9 amino acids, or 10 amino acids) in length may form the linkage between intracellular signaling sequences. In one embodiment, a glycine-serine doublet can be used as a suitable linker. In one embodiment, a single ammo acid, e.g., an alanine or a glycine, can be used as a suitable linker.

In some embodiments, the intracellular signaling domain comprises two or more costimulatory signaling domains, e.g., two costimulatory signaling domains, three costimulatory signaling domains, four costimulatory signaling domains, five costimulatory signaling domains, six costimulatory signaling domains, seven costimulatory signaling domains, eight costimulatory signaling domains, nine costimulatory signaling domains, 10 costimulatory signaling domains, or more costimulatory signaling domains. In one embodiment, the intracellular signaling domain comprises two costimulatory signaling domains. In some embodiments, the two or more costimulatory signaling domains are separated by a linker of the present disclosure (e.g., any of the linkers described in Table 1). In one embodiment, the linker is a glycine residue. In another embodiment, the linker is an alanine residue.

In some embodiments, a cell of the present disclosure expresses a CAR that includes an antigen-binding domain that binds a VSIG2, a transmembrane domain of the present disclosure, a primary signaling domain, and one or more costimulatory signaling domains.

In some embodiments, a cell of the present disclosure expresses an iCAR that includes an antigen-binding domain that binds VSIG2 (e.g., a VSIG2-specific antigen-binding domain having one or more of the amino acid sequences listed in Table A), a transmembrane domain of the present disclosure, and one or more intracellular inhibitory co-signaling domains. In some embodiments, a cell of the present disclosure expresses (1) a CAR that includes an antigen-binding domain that binds VSIG2 (e.g., a VSIG2-specific antigen-binding domain having one or more of the amino acid sequences listed in Table A), a transmembrane domain of the present disclosure, a primary signaling domain, and one or more costimulatory signaling domains.

### Natural Killer Cell Receptor (NKR) CARs

In some embodiments, a CAR of the present disclosure (e.g. the VSIG2-specific CARs described herein) comprises one or more components of a natural killer cell receptor (NKR), thereby forming an NKR-CAR. The NKR component may be a transmembrane domain, a hinge domain, or a cytoplasmic domain from any suitable natural killer cell receptor, including without limitation, a killer cell immunoglobulin-like receptor (KIR), such as KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIRS DPI; a natural cytotoxicity receptor (NCR), such as NKp30, NKp44, NKp46; a signaling lymphocyte activation molecule (SLAM) family of immune cell receptor, such as CD48, CD229, 2B4, CD84, NTB-A, CRACC, BLAME, and CD2F-10; an Fc receptor (FcR), such as CD16, and CD64; and an Ly49 receptor, such as LY49A and LY49C. In some embodiments, the NKR-CAR may interact with an adaptor molecule or intracellular signaling domain, such as DAP12. Exemplary configurations and sequences of CARs comprising NKR components are described in International Patent Publication WO2014/145252, published Sep. 18, 2014.

### Additional Chimeric Receptor Targets

Certain aspects of the present disclosure relate to chimeric receptors and polynucleotides that encode such chimeric receptors that bind to an antigen of interest in addition to VSIG2. Certain aspects of the present disclosure relate to chimeric receptors and cells, such as immunoresponsive cells, that have been genetically modified to express one or more of such chimeric receptors that bind to an antigen of interest in addition to VSIG2, and to methods of using such receptors and cells to treat and/or prevent malignancies, such as solid tumors, and other pathologies where an antigen-specific immune response is desired. Malignant cells have developed a series of mechanisms to protect themselves from immune recognition and elimination. The present disclosure provides immunogenicity within the tumor microenvironment for treating such malignant cells.

In some embodiments, a first chimeric receptor includes an antigen-binding domain that binds VSIG2 (e.g., a VSIG2-specific antigen-binding domain having one or more of the amino acid sequences listed in Table A) and a second chimeric receptor includes an additional antigen-binding domain that binds a second antigen, such as a tumor-associated antigen (e.g., a colorectal cancer-associated antigen). In some embodiments, a cell can express a first chimeric receptor specific for VSIG2 (e.g., a CAR including a VSIG2-specific antigen-binding domain having one or more of the amino acid sequences listed in Table A) and a second chimeric receptor specific for a second antigen, such as a tumor-associated antigen (e.g., a colorectal cancer-associated antigen). In some embodiments, a cell can express a first inhibitory chimeric receptor specific for VSIG2 (e.g., an inhibitory CAR including a VSIG2-specific antigen-binding domain having one or more of the amino acid sequences listed in Table A) and a second chimeric receptor specific for a second antigen, such as a tumor-associated antigen (e.g., a colorectal cancer-associated antigen). For example, a cell (e.g., an immunoresponsive cell) can be engineered to co-expresses or capable of co-expressing an iCAR that includes an antigen-binding domain that binds VSIG2 (e.g., a VSIG2-specific antigen-binding domain having one or more of the amino acid sequences listed in Table A) and an aCAR that targets a tumor-associated antigen (e.g., a colorectal cancer-associated antigen). Suitable antibodies that bind to an antigen in addition to VSIG2 include any antibody, whether natural or synthetic, full length or a fragment thereof, monoclonal or polyclonal, that binds sufficiently strongly and specifically to a second antigen, such a tumor-associated antigen (e.g., a colorectal cancer-associated antigen). In some embodiments, commercially available antibodies may be used for binding to a second antigen, such a tumor-associated antigen (e.g., a colorectal cancer-associated antigen). The CDRs of the commercially available antibodies are readily accessible by one skilled in the art using conventional sequencing technology. Further, one skilled in the art is able to construct polynucleotides encoding scFvs and chimeric receptors (e.g., CARs and TCRs) based on the CDRs of such commercially available antibodies.

### T Cell Receptor (TCR)

Certain aspects of the present disclosure relate to chimeric receptors that specifically bind to a second antigen, such a tumor-associated antigen (e.g., a colorectal cancer-associated antigen) and the chimeric receptor for the second antigen is an engineered T cell receptor (TCR). TCRs of the present disclosure are disulfide-linked heterodimeric proteins containing two variable chains expressed as part of a complex with the invariant CD3 chain molecules. TCRs are found on the surface of T cells, and are responsible for recognizing antigens as peptides bound to major histocompatibility complex (MHC) molecules. In certain embodiments, a TCR of the present disclosure comprises an alpha chain encoded by TRA and a beta chain encoded by TRB. In certain embodiments, a TCR comprises a gamma chain and a delta chain (encoded by TRG and TRD, respectively).

Each chain of a TCR is composed of two extracellular domains: a variable (V) region and a constant (C) region. The constant region is proximal to the cell membrane, followed by a transmembrane region and a short cytoplasmic tail. The variable region binds to the peptide/MHC complex. Each of the variable regions has three complementarity determining regions (CDRs).

In certain embodiments, a TCR can form a receptor complex with three dimeric signaling modules CD3δ/ε, CD3γ/ε, and CD247ζ/ζ or CD247ζ/η. When a TCR complex engages with its antigen and MHC (peptide/MHC), the T cell expressing the TCR complex is activated.

In some embodiments, a TCR of the present disclosure is a recombinant TCR. In certain embodiments, the TCR is a non-naturally occurring TCR. In certain embodiments, the TCR differs from a naturally occurring TCR by at least one amino acid residue. In some embodiments, the TCR differs from a naturally occurring TCR by at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, at least 13 amino acid residues, at least 14 amino acid residues, at least 15 amino acid residues, at least 20 amino acid residues, at least 25 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, or more amino acid residues. In certain embodiments, the TCR is modified from a naturally occurring TCR by at least one amino acid residue. In some embodiments, the TCR is modified from a naturally occurring TCR by at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, at least 13 amino acid residues, at least 14 amino acid residues, at least 15 amino acid residues, at least 20 amino acid residues, at least 25 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, or more amino acid residues.

### Chimeric TCRs

In some embodiments, a TCR of the present disclosure comprises one or more antigen-binding domains that may be grafted to one or more constant domain of a TCR chain, for example a TCR alpha chain or TCR beta chain, to create a chimeric TCR that binds specifically to a second antigen of interest, such a tumor-associated antigen (e.g., a colorectal cancer-associated antigen). Without wishing to be bound by theory, it is believed that chimeric TCRs may signal through the TCR complex upon antigen binding. For example, an antibody or antibody fragment (e.g., scFv) can be grafted to the constant domain, e.g., at least a portion of the extracellular constant domain, the transmembrane domain and the cytoplasmic domain, of a TCR chain, such as the TCR alpha chain and/or the TCR beta chain. As another example, the CDRs of an antibody or antibody fragment may be grafted into a TCR alpha chain and/or beta chain to create a chimeric TCR that binds specifically to a second antigen, such a tumor-associated antigen (e.g., a colorectal cancer-associated antigen). Such chimeric TCRs may be produced by methods known in the art (e.g., Willemsen R A et al., Gene Therapy 2000; 7:1369-1377; Zhang T et al., Cancer Gene Ther 2004 11: 487-496; and Aggen et al., Gene Ther. 2012 April; 19(4): 365-74).

### Immunoresponsive Cells

Certain aspects of the present disclosure relate to a cell, such as an immunoresponsive cell, that has been genetically engineered to comprise one or more chimeric receptors of the present disclosure or one or more polynucleotides encoding such chimeric receptors, and to methods of using such cells for treating solid tumors (e.g., colorectal cancer).

In some embodiments, the cell is a mammalian cell. In some embodiments, the mammalian cell is a primary cell. In some embodiments, the mammalian cell is a cell line. In some embodiments, the mammalian cell a bone marrow cell, a blood cell, a skin cell, bone cell, a muscle cell, a neuronal cell, a fat cell, a liver cell, or a heart cell. In some embodiments, the cell is a stem cell. Exemplary stem cells include, without limitation embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is a cell that is derived or differentiated from a stem cell of the present disclosure. In some embodiments, the cell is an immune cell. Immune cells of the present disclosure may be isolated or differentiated from a stem cell of the present disclosure (e.g., from an ESC or iPSC). Exemplary immune cells include, without limitation, T cells (e.g., helper T cells, cytotoxic T cells, memory T cells, regulatory T cells, natural killer T cells, alpha beta T cells, and gamma delta T cells), B cells, natural killer (NK) cells, dendritic cells, myeloid cells, macrophages, and monocytes. In some embodiments, the cell is a neuronal cell. Neuronal cells of the present disclosure may be isolated or differentiated from a stem cell of the present disclosure (e.g., from an ESC or iPSC). Exemplary neuronal cells include, without limitation, neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the cell is an immunoresponsive cell. Immunoresponsive cells of the present disclosure may be isolated or differentiated from a stem cell of the present disclosure (e.g., from an ESC or iPSC). Exemplary immunoresponsive cells of the present disclosure include, without limitation, cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Examples of immunoresponsive cells of the lymphoid lineage include, without limitation, T cells, Natural Killer (NK) cells, embryonic stem cells, pluripotent stem cells, and induced pluripotent stem cells (e.g., those from which lymphoid cells may be derived or differentiated). T cells can be lymphocytes that mature in the thymus and are chiefly responsible for cell-mediated immunity. T cells are involved in the adaptive immune system. In some embodiments, T cells of the present disclosure can be any type of T cells, including, without limitation, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of one or more chimeric receptors, such as a chimeric TCRs or CARs.

Natural killer (NK) cells can be lymphocytes that are part of cell-mediated immunity and act during the innate immune response. NK cells do not require prior activation in order to perform their cytotoxic effect on target cells.

In some embodiments, an immunoresponsive cell of the present disclosure is a T cell. T cells of the present disclosure may be autologous, allogeneic, or derived in vitro from engineered progenitor or stem cells.

In some embodiments, an immunoresponsive cell of the present disclosure is a universal T cell with deficient TCR-αβ. Methods of developing universal T cells are described in the art, for example, in Valton et al., Molecular Therapy (2015); 23 9, 1507-1518, and Torikai et al., Blood 2012 119:5697-5705.

In some embodiments, an immunoresponsive cell of the present disclosure is an isolated immunoresponsive cell comprising one or more chimeric receptors of the present disclosure. In some embodiments, the immunoresponsive cell comprises one or more, two or more, three or more, four or more, five or more, six or more, seven or more, eight or more, nine or more, or ten or more chimeric receptors of the present disclosure.

In some embodiments, an immunoresponsive cell is a T cell. In some embodiments, an immunoresponsive cell is a Natural Killer (NK) cell.

In some embodiments, an immunoresponsive cell express or is capable of expressing an immune receptor. Immune receptors generally are capable of inducing signal transduction or changes in protein expression in the immune receptor-expressing cell that results in the modulation of an immune response upon binding to a cognate ligand (e.g., regulate, activate, initiate, stimulate, increase, prevent, attenuate, inhibit, reduce, decrease, inhibit, or suppress an immune response). For example, when CD3 chains present in a TCR/CAR cluster in response to ligand binding, an immunoreceptor tyrosine-based activation motifs (ITAMs)-meditated signal transduction cascade is produced. Specifically, in certain embodiments, when an endogenous TCR, exogenous TCR, chimeric TCR, or a CAR (specifically an activating CAR) binds their respective antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated that in turn can initiate a T cell activation pathway and ultimately activates transcription factors, such as NF-κB and AP-1. These transcription factors are capable of inducing global gene expression of the T cell to increase IL-2 production for proliferation and expression of master regulator T cell proteins in order to initiate a T cell mediated immune response, such as cytokine production and/or T cell mediated killing.

### Cells Expressing Multiple Chimeric Receptors

In some embodiments, a cell of the present disclosure (e.g., an immunoresponsive cell) comprises two or more chimeric receptors of the present disclosure. In some embodiments, the cell comprises two or more chimeric receptors, wherein one of the two or more chimeric receptors is a chimeric inhibitory receptor. In some embodiments, the cell comprises three or more chimeric receptors, wherein at least one of the three or more chimeric receptors is a chimeric inhibitory receptor. In some embodiments, the cell comprises four or more chimeric receptors, wherein at least one of the four or more chimeric receptors is a chimeric inhibitory receptor. In some embodiments, the cell comprises five or more chimeric receptors, wherein at least one of the five or more chimeric receptors is a chimeric inhibitory receptor.

In some embodiments, each of the two or more chimeric receptors comprise a different antigen-binding domain, e.g., that binds to the same antigen or to a different antigen. In some embodiments each antigen bound by the two or more chimeric receptors are expressed on the same cell, such as an epithelial cell type (e.g., same epithelial cell type).

In embodiments where a cell of the present disclosure (e.g., an immunoresponsive cell) expresses two or more distinct chimeric receptors, the antigen-binding domain of each of the different chimeric receptors may be designed such that the antigen-binding domains do not interact with one another. For example, a cell of the present disclosure (e.g., an immunoresponsive cell) expressing a first chimeric receptor (e.g., a VSIG2-specific chimeric receptor) and a second chimeric receptor may comprise a first chimeric receptor that comprises an antigen-binding domain that does not form an association with the antigen-binding domain of the second chimeric receptor. For example, the antigen-binding domain of the first chimeric receptor may comprise an antibody fragment, such as an scFv, while the antigen-binding domain of the second chimeric receptor may comprise a VHH.

Without wishing to be bound by theory, it is believed that in cells having a plurality of chimeric membrane embedded receptors that each comprise an antigen-binding domain, interactions between the antigen-binding domains of each of the receptors can be undesirable, because such interactions may inhibit the ability of one or more of the antigen-binding domains to bind their cognate antigens. Accordingly, in embodiments where cells of the present disclosure (e.g., immunoresponsive cells) express two or more chimeric receptors, the chimeric receptors comprise antigen-binding domains that minimize such inhibitory interactions. In one embodiment, the antigen-binding domain of one chimeric receptor comprises an scFv and the antigen-binding domain of the second chimeric receptor comprises a single VH domain, e.g., a camelid, shark, or lamprey single VH domain, or a single VH domain derived from a human or mouse sequence.

In some embodiments, when present on the surface of a cell, binding of the antigen-binding domain of the first chimeric receptor to its cognate antigen (e.g., a VSIG2-specific chimeric receptor binding to VSIG2) is not substantially reduced by the presence of the second chimeric receptor. In some embodiments, binding of the antigen-binding domain of the first chimeric receptor to its cognate antigen in the presence of the second chimeric receptor is 85%, 90%, 95%, 96%, 97%, 98%, or 99% of binding of the antigen-binding domain of the first chimeric receptor to its cognate antigen in the absence of the second chimeric receptor. In some embodiments, when present on the surface of a cell, the antigen-binding domains of the first chimeric receptor and the second chimeric receptor associate with one another less than if both were scFv antigen-binding domains. In some embodiments, the antigen-binding domains of the first chimeric receptor and the second chimeric receptor associate with one another 85%, 90%, 95%, 96%, 97%, 98%, or 99% less than if both were scFv antigen-binding domains.

### Chimeric Inhibitory Receptors

In some embodiments, a cell of the present disclosure (e.g., an immunoresponsive cell) comprises one or more chimeric inhibitory receptors of the present disclosure. In some embodiments, each of the one or more chimeric inhibitory receptors comprises an antigen-binding domain that binds an antigen generally expressed on normal cells (e.g., cells generally considered to be healthy) but not on tumor cells, such as colorectal cancer cells. In some embodiments, a chimeric inhibitory receptor includes an antigen-binding domain that binds VSIG2 (e.g., a VSIG2-specific antigen-binding domain having one or more of the amino acid sequences listed in Table A).

In some embodiments, the one or more chimeric inhibitory receptors bind antigens that are expressed on a non-tumor cell derived from a tissue selected from brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor (e.g. a VSIG2-specific chimeric inhibitory receptor) may be used, for example, with one or more activating chimeric receptors (e.g., activating chimeric TCRs or CARs) expressed on a cell of the present disclosure (e.g., an immunoresponsive cell) as NOT-logic gates to control, modulate, or otherwise inhibit one or more activities of the one or more activating chimeric receptors. In some embodiments, a chimeric inhibitory receptor of the present disclosure may inhibit one or more activities of a cell of the present disclosure (e.g., an immunoresponsive cell).

### Co-Stimulatory Ligands

In some embodiments, a cell of the present disclosure (e.g., an immunoresponsive cell) can further include one or more recombinant or exogenous co-stimulatory ligands. For example, the cell can be further transduced with one or more co-stimulatory ligands, such that the cell co-expresses or is induced to co-express one or more chimeric receptors of the present disclosure (e.g., the VSIG2-specific CARs described herein) and one or more co-stimulatory ligands. Without wishing to be bound by theory, it is believed that the interaction between the one or more chimeric receptors and the one or more co-stimulatory ligands may provide a non-antigen-specific signal important for full activation of the cell. Examples of suitable co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Examples of suitable TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14). The immunoglobulin (Ig) superfamily is a large group of cell surface and soluble proteins that are involved in the recognition, binding, or adhesion processes of cells. These proteins share structural features with immunoglobulins and possess an immunoglobulin domain (fold). Examples of suitable immunoglobulin superfamily ligands include, without limitation, CD80 and CD86, both ligands for CD28, PD-L1/(B7-H1) that ligands for PD-1. In certain embodiments, the one or more co-stimulatory ligands are selected from 4-1BBL, CD80, CD86, CD70, OX40L, CD48, TNFRSF14, PD-L1, and combinations thereof.

### Chemokine Receptor

In some embodiments, a cell of the present disclosure (e.g., an immunoresponsive cell) comprises one or more chimeric receptors (e.g. the VSIG2-specific CARs described herein) and may further include one or more chemokine receptors. For example, transgenic expression of chemokine receptor CCR2b or CXCR2 in cells, such as T cells, enhances trafficking to CCL2-secreting or CXCL1-secreting solid tumors (Craddock et al, J Immunother. 2010 October; 33(8):780-8 and Kershaw et al. Hum Gene Ther. 2002 Nov. 1; 13(16): 1971-80). Without wishing to be bound by theory, it is believed that chemokine receptors expressed on chimeric receptor-expressing cells of the present disclosure may recognize chemokines secreted by tumors and improve targeting of the cell to the tumor, which may facilitate the infiltration of the cell to the tumor and enhance the antitumor efficacy of the cell. Chemokine receptors of the present disclosure may include a naturally occurring chemokine receptor, a recombinant chemokine receptor, or a chemokine-binding fragment thereof. Examples of suitable chemokine receptors that may expressed on a cell of the present disclosure include, without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In some embodiments, the chemokine receptor to be expressed on the cell is chosen based on the chemokines secreted by the tumor.

### Chimeric Receptor Regulation

Some embodiments of the present disclosure relate to regulating one or more chimeric receptor activities of chimeric receptor-expressing cells of the present disclosure (e.g. the VSIG2-specific CARs described herein). There are several ways chimeric receptor activities can be regulated. In some embodiments, a regulatable chimeric receptor, wherein one or more chimeric receptor activities can be controlled, may be desirable to optimize the safety and/or efficacy of the chimeric receptor therapy. For example, inducing apoptosis using a caspase fused to a dimerization domain (see, e.g., Di et al., N Engl. J. Med. 2011 Nov. 3; 365(18): 1673-1683) can be used as a safety switch in the chimeric receptor therapy. In some embodiments, a chimeric receptor-expressing cell of the present disclosure can also express an inducible Caspase-9 (iCaspase-9) that, upon administration of a dimerizer drug, such as rimiducid (IUPAC name: [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl)butanoyl]piperidine-2-carboxylate), induces activation of the Caspase-9 and results in apoptosis of the cells. In some embodiments, the iCaspase-9 contains a binding domain that comprises a chemical inducer of dimerization (CID) that mediates dimerization in the presence of the CID, which results in inducible and selective depletion of the chimeric receptor-expressing cells.

Alternatively, in some embodiments a chimeric receptor of the present disclosure may be regulated by utilizing a small molecule or an antibody that deactivates or otherwise inhibits chimeric receptor activity. For example, an antibody may delete the chimeric receptor-expressing cells by inducing antibody dependent cell-mediated cytotoxicity (ADCC). In some embodiments, a chimeric receptor-expressing cell of the present disclosure may further express an antigen that is recognized by a molecule that is capable of inducing cell death by ADCC or complement-induced cell death. For example, a chimeric receptor-expressing cell of the present disclosure may further express a receptor capable of being targeted by an antibody or antibody fragment. Examples of suitable receptors that may be targeted by an antibody or antibody fragment include, without limitation, EpCAM, VEGFR, integrins (e.g., αvβ3, α4, αi¾β3, α4β7, α5β1, αvβ3, αv), members of the TNF receptor superfamily (e.g., TRAIL-R1 and TRAIL-R2), PDGF receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof.

In some embodiments, a chimeric receptor-expressing cell of the present disclosure may also express a truncated epidermal growth factor receptor (EGFR) that lacks signaling capacity but retains an epitope that is recognized by molecules capable of inducing ADCC (e.g., WO2011/056894).

In some embodiments, a chimeric receptor-expressing cell of the present disclosure further includes a highly expressing compact marker/suicide gene that combines target epitopes from both CD32 and CD20 antigens in the chimeric receptor-expressing cell, which binds an anti-CD20 antibody (e.g., rituximab) resulting in selective depletion of the chimeric receptor-expressing cell by ADCC. Other methods for depleting chimeric receptor-expressing cells of the present disclosure my include, without limitation, administration of a monoclonal anti-CD52 antibody that selectively binds and targets the chimeric receptor-expressing cell for destruction by inducing ADCC. In some embodiments, the chimeric receptor-expressing cell can be selectively targeted using a chimeric receptor ligand, such as an anti-idiotypic antibody. In some embodiments, the anti-idiotypic antibody can cause effector cell activity, such as ADCC or ADC activity. In some embodiments, the chimeric receptor ligand can be further coupled to an agent that induces cell killing, such as a toxin. In some embodiments, a chimeric receptor-expressing cell of the present disclosure may further express a target protein recognized by a cell depleting agent of the present disclosure. In some embodiments, the target protein is CD20 and the cell depleting agent is an anti-CD20 antibody. In such embodiments, the cell depleting agent is administered once it is desirable to reduce or eliminate the chimeric receptor-expressing cell. In some embodiments, the cell depleting agent is an anti-CD52 antibody.

In some embodiments, a regulated chimeric receptor comprises a set of polypeptides, in which the components of a chimeric receptor of the present disclosure are partitioned on separate polypeptides or members. For example, the set of polypeptides may include a dimerization switch that, when in the presence of a dimerization molecule, can couple the polypeptides to one another to form a functional chimeric receptor.

### Chimeric Receptor-Encoding Polynucleotide Constructs

Certain aspects of the present disclosure relate to polynucleotides (e.g., isolated polynucleotides) encoding one or more chimeric receptors of the present disclosure (e.g. the VSIG2-specific CARs described herein). In some embodiments, the polynucleotide is an RNA construct, such as a messenger RNA (mRNA) transcript or a modified RNA. In some embodiments, the polynucleotide is a DNA construct.

In some embodiments, a polynucleotide of the present disclosure encodes a chimeric receptor that comprises one or more antigen-binding domain, where each domain binds to a target antigen (e.g., VSIG2), a transmembrane domain, and one or more intracellular signaling domains. In some embodiments, the polynucleotide encodes a chimeric receptor that comprises an antigen-binding domain, a transmembrane domain, a primary signaling domain (e.g., CD3-zeta domain), and one or more costimulatory signaling domains. In some embodiments, the polynucleotide further comprises a nucleic acid sequence encoding a spacer region. In some embodiments, the antigen-binding domain is connected to the transmembrane domain by the spacer region. In some embodiments, the spacer region comprises a nucleic acid sequence selected from any of the nucleic acid sequences listed in Table 3. In some embodiments, the nucleic acid further comprises a nucleotide sequence encoding a leader sequence.

The polynucleotides of the present disclosure may be obtained using any suitable recombinant methods known in the art, including, without limitation, by screening libraries from cells expressing the gene of interest, by deriving the gene of interest from a vector known to include the gene, or by isolating the gene of interest directly from cells and tissues containing the gene using standard techniques. Alternatively, the gene of interest may be produced synthetically.

In some embodiments, a polynucleotide of the present disclosure in comprised within a vector. In some embodiments, a polynucleotide of the present disclosure is expressed in a cell via transposons, a CRISPR/Cas9 system, a TALEN, or a zinc finger nuclease.

In some embodiments, expression of a polynucleotide encoding a chimeric receptor of the present disclosure may be achieved by operably linking the nucleic acid to a promoter and incorporating the construct into an expression vector. A suitable vector can replicate and integrate in eukaryotic cells. Typical cloning vectors contain transcription and translation terminators, initiation sequences, and promoters useful for regulating expression of the desired nucleic acid.

In some embodiments, expression constructs of the present disclosure may also be used for nucleic acid immunization and gene therapy, using standard gene delivery protocols (e.g., U.S. Pat. Nos. 5,399,346, 5,580,859, and 5,589,466). In some embodiments, a vector of the present disclosure is a gene therapy vector.

A polynucleotide of the present disclosure can be cloned into a number of types of vectors. For example, the polynucleotide can be cloned into a vector including, without limitation, a plasmid, a phagemid, a phage derivative, an animal virus, or a cosmid. In some embodiments, the vector may be an expression vector, a replication vector, a probe generation vector, or a sequencing vector.

In some embodiments, the plasmid vector comprises a transposon/transposase system to incorporate the polynucleotides of the present disclosure into the host cell genome. Methods of expressing proteins in immune cells using a transposon and transposase plasmid system are generally described in Chicaybam L, Hum Gene Ther. 2019 April; 30(4):511-522. doi: 10.1089/hum.2018.218; and Ptáĉková P, Cytotherapy. 2018 April; 20(4):507-520. doi: 10.1016/j.jcyt.2017.10.001, each of which are hereby incorporated by reference in their entirety. In some embodiments, the transposon system is the Sleeping Beauty transposon/transposase or the piggyBac transposon/transposase.

In some embodiments, an expression vector of the present disclosure may be provided to a cell in the form of a viral vector. Suitable viral vector systems are well known in the art. For example, viral vectors may be derived from retroviruses, adenoviruses, adeno-associated viruses, herpes viruses, and lentiviruses. In some embodiments, a vector of the present disclosure is a lentiviral vector. Lentiviral vectors are suitable for long-term gene transfer as such vectors allow long-term, stable integration of a transgene and its propagation in daughter cells. Lentiviral vectors are also advantageous over vectors derived from onco-retroviruses (e.g., murine leukemia viruses) in that lentiviral vectors can transduce non-proliferating cells. In some embodiments, a vector of the present disclosure is an adenoviral vector (A5/35). In some embodiments, a vector of the present disclosure contains an origin of replication functional in at least one organism, a promoter sequence, convenient restriction endonuclease sites, and one or more selectable markers (e.g., WO01/96584; WO01/29058; and U.S. Pat. No. 6,326,193). A number of viral based systems have been developed for gene transfer into mammalian cells. A selected gene can be inserted into a vector and packaged in retroviral particles using techniques known in the art. The recombinant virus can then be isolated and delivered to mammalian cells either in vivo or ex vivo. A number of retroviral systems are known in the art.

In some embodiments, vectors of the present disclosure include additional promoter elements, such as enhancers that regulate the frequency of transcriptional initiation. Enhancers are typically located in a region that is 30 bp to 110 bp upstream of the start site, although a number of promoters have been shown to contain functional elements downstream of the start site as well. The spacing between promoter elements may be flexible, so that promoter function is preserved when elements are inverted or moved relative to one another. For example, in the thymidine kinase (tk) promoter the spacing between promoter elements can be increased to 50 bp apart before activity begins to decline. Depending on the promoter, individual elements may function either cooperatively or independently to activate transcription. Exemplary promoters may include, without limitation, the SFFV gene promoter, the EFS gene promoter, the CMV IE gene promoter, the EF1a promoter, the ubiquitin C promoter, and the phosphoglycerokinase (PGK) promoter.

In some embodiments, a promoter that is capable of expressing a polynucleotide of the present disclosure in a mammalian cell, such as an immunoresponsive cell of the present disclosure, is the EF1a promoter. The native EF1a promoter drives expression of the alpha subunit of the elongation factor-1 complex, which is responsible for the enzymatic delivery of aminoacyl tRNAs to the ribosome. The EF1a promoter has been widely used in mammalian expression plasmids and has been shown to be effective in driving chimeric receptor expression from polynucleotide cloned into a lentiviral vector.

In some embodiments, a promoter that is capable of expressing a polynucleotide of the present disclosure in a mammalian cell, such as an immunoresponsive cell of the present disclosure, is a constitutive promoter. For example, a suitable constitutive promoter is the spleen focus forming virus (SFFV) promoter. Another example of a suitable constitutive promoter is the immediate early cytomegalovirus (CMV) promoter. The CMV promoter is a strong constitutive promoter that is capable of driving high levels of expression of any polynucleotide sequence operatively linked to the promoter. Other suitable constitutive promoters include, without limitation, a ubiquitin C (UbiC) promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, an actin promoter, a myosin promoter, an elongation factor-1a promoter, a hemoglobin promoter, and a creatine kinase promoter.

In some embodiments, a promoter that is capable of expressing a polynucleotide of the present disclosure in a mammalian cell, such as an immunoresponsive cell of the present disclosure, is an inducible promoter. Use of an inducible promoter may provide a molecular switch that is capable of inducing or repressing expression of a polynucleotide of the present disclosure when the promoter is operatively linked to the polynucleotide. Examples of inducible promoters include, without limitation, a metallothionine promoter, a glucocorticoid promoter, a progesterone promoter, and a tetracycline promoter.

In some embodiments, a vector of the present disclosure may further comprise a signal sequence to facilitate secretion, a polyadenylation signal and transcription terminator, an element allowing episomal replication, and/or elements allowing for selection.

In some embodiments, a vector of the present disclosure can further comprise a selectable marker gene and/or reporter gene to facilitate identification and selection of chimeric receptor-expressing cells from a population of cells that have been transduced with the vector. In some embodiments, the selectable marker may be encoded by a polynucleotide that is separate from the vector and used in a co-transfection procedure. Either selectable marker or reporter gene may be flanked with appropriate regulator sequences to allow expression in host cells. Examples of selectable markers include, without limitation, antibiotic-resistance genes, such as neo and the like.

In some embodiments, reporter genes may be used for identifying transduced cells and for evaluating the functionality of regulatory sequences. As disclosed herein, a reporter gene is a gene that is not present in or expressed by the recipient organism or tissue and that encodes a polypeptide whose expression results in an easily detectable property, such as enzymatic activity. Expression of the reporter gene can be assayed at a suitable time after the polynucleotide has been introduced into the recipient cells. Examples of reporter genes include, without limitation, genes encoding for luciferase, genes encoding for beta-galactosidase, genes encoding for chloramphenicol acetyl transferase, genes encoding for secreted alkaline phosphatase, and genes encoding for green fluorescent protein. Suitable expression systems are well known in the art and may be prepared using known techniques or obtained commercially. In some embodiments, a construct with a minimal 5′ flanking region showing the highest level of expression of the reporter gene is identified as the promoter. Such promoter regions may be linked to a reporter gene and used to evaluate agents for the ability to modulate promoter-driven transcription.

In some embodiments, a vector comprising a polynucleotide sequence encoding a chimeric receptor of the present disclosure further comprises a second polynucleotide encoding a polypeptide that increases the activity of the chimeric receptor.

In embodiments where a chimeric receptor-expressing cell comprises two or more chimeric receptors, a single polynucleotide may encode the two or more chimeric receptors under a single regulatory control element (e.g., promoter) or under separate regulatory control elements for each chimeric receptor-encoding nucleotide sequence comprised in the polynucleotide. In some embodiments where a chimeric receptor-expressing cell comprises two or more chimeric receptors, each chimeric receptor may be encoded by a separate polynucleotide. In some embodiments, each separate polynucleotide comprises its own control element (e.g., promoter). In some embodiments, a single polynucleotide encodes the two or more chimeric receptors and the chimeric receptor-encoding nucleotide sequences are in the same reading frame and are expressed as a single polypeptide chain. In such embodiments, the two or more chimeric receptors may be separated by one or more peptide cleavage sites, such as auto-cleavage sites or substrates for an intracellular protease. Suitable peptide cleavage sites may include, without limitation, a T2A peptide cleavage site, a P2A peptide cleavage site, an E2A peptide cleavage sire, and an F2A peptide cleavage site. In some embodiments, the two or more chimeric receptors comprise a T2A peptide cleavage site. In some embodiments, the two or more chimeric receptors comprise an E2A peptide cleavage site. In some embodiments, the two or more chimeric receptors comprise a T2A and an E2A peptide cleavage site.

Methods of introducing and expressing genes into a cell are well known in the art. For example, in some embodiments, an expression vector can be transferred into a host cell by physical, chemical, or biological means. Examples of physical means for introducing a polynucleotide into a host cell include, without limitation, calcium phosphate precipitation, lipofection, particle bombardment, microinjection, and electroporation. Examples of chemical means for introducing a polynucleotide into a host cell include, without limitation, colloidal dispersion systems, macromolecule complexes, nanocapsules, microspheres, beads, and lipid-based systems including oil-in-water emulsions, micelles, mixed micelles, and liposomes. Examples of biological means for introducing a polynucleotide into a host cell include, without limitation, the use of DNA and RNA vectors.

In some embodiments, liposomes may be used as a non-viral delivery system to introduce a polynucleotide or vector of the present disclosure into a host cell in vitro, ex vivo, or in vivo. In some embodiments, the polynucleotide may be associated with a lipid, for example by being encapsulated in the aqueous interior of a liposome, being interspersed within the lipid bilayer of a liposome, being attached to a liposome via a linking molecule that is associated with both the liposome and the polynucleotide, being entrapped in a liposome, being complexed with a liposome, being dispersed in a solution containing a lipid, being mixed with a lipid, being combined with a lipid, being contained as a suspension in a lipid, being contained or complexed with a micelle, or otherwise being associated with a lipid. As disclosed herein, lipid-associated polynucleotide or vector compositions are not limited to any particular structure in solution. In some embodiments, such compositions may be present in a bilayer structure, as micelles or with a “collapsed” structure. Such compositions may also be interspersed in a solution, forming aggregates that are not uniform in size or shape. As disclosed herein, lipids are fatty substances that may be naturally occurring or synthetic. In some embodiments, lipids can include the fatty droplets that naturally occur in the cytoplasm or the class of compounds that contain long-chain aliphatic hydrocarbons and their derivatives, such as fatty acids, alcohols, amines, amino alcohols, and aldehydes. Suitable lipids may be obtained from commercial sources and include, without limitation, dimyristyl phosphatidylcholine (“DMPC”), dicetylphosphate (“DCP”), cholesterol, and dimyristylphosphatidylglycerol (“DMPG”). Stock solutions of lipids in chloroform or chloroform/methanol can be stored at about −20° C. Chloroform is used as the solvent, as it is more readily evaporated than methanol. As used herein, a “liposome” may encompass a variety of single and multilamellar lipid vehicles formed by the generation of enclosed lipid bilayers or aggregates. In some embodiments, liposomes can be characterized as having vesicular structures with a phospholipid bilayer membrane and an inner aqueous medium. In some embodiments, multilamellar liposomes may have multiple lipid layers separated by aqueous medium. Multilamellar liposomes can form spontaneously when phospholipids are suspended in an excess of aqueous solution. In some embodiments, lipid components may undergo self-rearrangement before the formation of closed structures and can entrap water and dissolved solutes between the lipid bilayers. In some embodiments, the lipids may assume a micellar structure or merely exist as nonuniform aggregates of lipid molecules.

In some embodiments, a polynucleotide or vector of the present disclosure is introduced into a mammalian host cell, such as an immunoresponsive cell of the present disclosure. In some embodiments, the presence of a polynucleotide or vector of the present disclosure in a host cell may be confirmed by any suitable assay known in the art, including without limitation Southern blot assays, Northern blot assays, RT-PCR, PCR, ELISA assays, and Western blot assays.

In some embodiments, a polynucleotide or vector of the present disclosure is stably transduced into an immunoresponsive cell of the present disclosure. In some embodiments, cells that exhibit stable expression of the polynucleotide or vector express the encoded chimeric receptor for at least 1 week, at least 2 weeks, at least 3 weeks, at least 4 weeks, at least 5 weeks, at least 6 weeks, at least 7 weeks, at least 8 weeks, at least 3 months, at least 6 months, at least 9 months, or at least 12 months after transduction.

In embodiments where a chimeric receptor of the present disclosure is transiently expressed in a cell, a chimeric receptor-encoding polynucleotide or vector of the present disclosure is transfected into an immunoresponsive cell of the present disclosure. In some embodiments the immunoresponsive cell expresses the chimeric receptor for about 4 days, about 5 days, about 6 days, about 7 days, about 8 days, about 9 days, about 10 days, about 11 days, about 12 days, about 13 days, about 14 days, or about 15 days after transfection.

In some embodiments, the polynucleotide construct encodes a bicistronic chimeric antigen receptor. In some embodiments, the encoded bicistronic chimeric antigen receptor comprises a VSIG2 CAR (such as a VSIG2 inhibitory CAR) and a CAR specific for a second antigen (such as a tumor-targeting chimeric receptor).

In some embodiments, the polynucleotide construct encodes a bivalent chimeric antigen receptor. In some embodiments, the encoded bivalent chimeric antigen receptor comprises a VSIG2 antigen binding domain and a second antigen binding domain.

### Pharmaceutical Compositions and Administration

Certain aspects of the present disclosure relate to compositions (e.g., pharmaceutical compositions) comprising one or more chimeric receptors of the present disclosure or immunoresponsive cells of the present disclosure that express such one or more chimeric receptors. In some embodiments, compositions comprising chimeric receptors or genetically modified immunoresponsive cells that express such chimeric receptors can be provided systemically or directly to a subject for the treatment of a proliferative disorder, such as a myeloid disorder. In certain embodiments, the composition is directly injected into an organ of interest (e.g., an organ affected by a disorder). Alternatively, the composition may be provided indirectly to the organ of interest, for example, by administration into the circulatory system (e.g., the tumor vasculature). Expansion and differentiation agents can be provided prior to, during, or after administration of the composition to increase production of T cells, NK cells, or CTL cells in vitro or in vivo.

Compositions comprising genetically modified cells of the present disclosure may be administered in any physiologically acceptable vehicle, for example intravascularly, although they may also be introduced into bone or other convenient sites where the genetically modified cells may find an appropriate site for regeneration and differentiation (e.g., thymus). In some embodiments, at least 1×10⁵ cells may be administered, eventually reaching 1×10¹⁰ or more cells. Compositions comprising genetically modified cells of the present disclosure can comprise a purified population of cells. Methods for determining the percentage of genetically modified cells in a population of cells are well known in the art and include, without limitation, fluorescence activated cell sorting (FACS). In some embodiments, the purity of genetically modified cells in a population of cells may be about 50%, about 55%, about 60%, or about 65%, about 70%, about 75%, about 80%, about 85%, about 90%, about 95%, about 97%, about 98%, about 99%, or more of the cells in the population of cells. Dosages can be readily adjusted by those skilled in the art (e.g., a decrease in purity may require an increase in dosage). The cells can be introduced by injection, catheter, or the like. In some embodiments, factors can also be included, for example, IL-2, IL-3, IL-6, IL-11, IL-7, IL-12, IL-15, IL-21, G-CSF, MCSF, GM-CSF, gamma-interferon, and erythropoietin.

In certain embodiments, the compositions are pharmaceutical compositions comprising genetically modified cells, such as immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. In some embodiments, immunoresponsive cells of the present disclosure or their progeny may be derived from peripheral blood cells (e.g., in vivo, ex vivo, or in vitro derived) and may be administered via localized injection, including catheter administration, systemic injection, localized injection, intravenous injection, or parenteral administration. When administering a therapeutic composition of the present disclosure (e.g., a pharmaceutical composition containing a genetically modified cell of the present disclosure), it will generally be formulated in a unit dosage injectable form (solution, suspension, emulsion).

### Formulations

Certain aspects of the present disclosure relate to formulations of compositions comprising chimeric receptors of the present disclosure or genetically modified cells (e.g., immunoresponsive cells of the present disclosure) expressing such chimeric receptors. In some embodiments, compositions of the present disclosure comprising genetically modified cells may be provided as sterile liquid preparations, including without limitation isotonic aqueous solutions, suspensions, emulsions, dispersions, and viscous compositions, which may be buffered to a selected pH. Liquid preparations are typically easier to prepare than gels, other viscous compositions, and solid compositions. Additionally, liquid compositions may be more convenient to administer, especially by injection. In some embodiments, viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (e.g., glycerol, propylene glycol, liquid polyethylene glycol, etc.) and suitable mixtures thereof.

In some embodiments, sterile injectable solutions can be prepared by incorporating genetically modified cells of the present disclosure in a sufficient amount of the appropriate solvent with various amounts of any other ingredients, as desired. Such compositions may be in admixture with a suitable carrier, diluent, or excipient such as sterile water, physiological saline, glucose, dextrose, or the like. In some embodiments, the compositions can also be lyophilized. The compositions can contain auxiliary substances such as wetting, dispersing agents, pH buffering agents, and antimicrobials depending upon the route of administration and the preparation desired.

In some embodiments, compositions of the present disclosure may further include various additives that may enhance the stability and sterility of the compositions. Examples of such additives include, without limitation, antimicrobial preservatives, antioxidants, chelating agents, and buffers. In some embodiments, microbial contamination may be prevented by the inclusions of any of various antibacterial and antifungal agents, including without limitation parabens, chlorobutanol, phenol, sorbic acid, and the like. Prolonged absorption of an injectable pharmaceutical formulation of the present disclosure can be brought about by the use of suitable agents that delay absorption, such as aluminum monostearate and gelatin.

In some embodiments, compositions of the present disclosure can be isotonic, i.e., having the same osmotic pressure as blood and lacrimal fluid. In some embodiments, the desired isotonicity may be achieved using, for example, sodium chloride, dextrose, boric acid, sodium tartrate, propylene glycol, or other inorganic or organic solutes.

In some embodiments, the components of the formulations of the present disclosure are selected to be chemically inert and to not affect the viability or efficacy of the genetically modified cells of the present disclosure.

One consideration concerning the therapeutic use of the genetically modified cells of the present disclosure is the quantity of cells needed to achieve optimal efficacy. In some embodiments, the quantity of cells to be administered will vary for the subject being treated. In certain embodiments, the quantity of genetically modified cells that are administered to a subject in need thereof may range from 1×10⁴ cells to 1×10¹⁰ cells. In some embodiments, the precise quantity of cells that would be considered an effective dose may be based on factors individual to each subject, including their size, age, sex, weight, and condition of the particular subject. Dosages can be readily ascertained by those skilled in the art based on the present disclosure and the knowledge in the art.

### Heterologous Moieties and Modifications

In a further series of embodiments, the VSIG2-specific chimeric proteins herein (e.g. a VSIG2-specific chimeric protein including an antigen-binding domain having one or more of the amino acid sequences listed in Table A) include additional moieties and/or modifications.

### Drug Conjugates

In various embodiments, the VSIG2-specific chimeric protein includes an antigen-binding domain having one or more of the amino acid sequences listed in Table A conjugated to a therapeutic agent (i.e. drug) to form an antibody-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the antigen-binding domain through a linker peptide, as discussed in more detail herein.

Methods of preparing antibody-drug conjugates (ADCs) that can be adapted to conjugate drugs to the antigen-binding domains disclosed herein (e.g., having one or more of the amino acid sequences listed in Table A) are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015). Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### Additional Binding Moieties

In various embodiments, the VSIG2-specific chimeric protein includes an antigen-binding domain having one or more of the amino acid sequences listed in Table A and one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of one or more peptides of the VSIG2-specific antigen-binding domain, such as the VH and/or VL, Fab heavy and/or light-chain fragment, or scFv. In particular embodiments, the one or more additional binding moieties are attached to the N-terminus of one or more peptides of the VSIG2-specific antigen-binding domain, such as the VH and/or VL, Fab heavy and/or light-chain fragment, or scFv.

In certain embodiments, the one or more additional binding moieties are specific for a different antigen or epitope than VSIG2. In certain embodiments, the one or more additional binding moieties are specific for VSIG2.

In certain embodiments, the one or more additional binding moieties are attached to the antigen-binding domains described herein (e.g., having one or more of the amino acid sequences listed in Table A) using in vitro methods including, but not limited to, reactive chemistry (e.g., Click-chemistry) and affinity tagging systems. In certain embodiments, the one or more additional binding moieties are attached to the antigen-binding domains described herein (e.g., having one or more of the amino acid sequences listed in Table A) through Fc-mediated binding (e.g. Protein A/G). In certain embodiments, the one or more additional binding moieties are attached to the antigen-binding domains described herein (e.g., having one or more of the amino acid sequences listed in Table A) using recombinant DNA techniques, such as encoding the nucleotide sequence of the fusion product between the antigen-binding domains described herein and the additional binding moieties on the same expression vector (e.g. plasmid).

### Functional/Reactive Groups

In various embodiments, the antigen-binding domains described herein (e.g., having one or more of the amino acid sequences listed in Table A) have modifications that comprise functional groups or chemically reactive groups that can be used in downstream processes, such as linking to additional moieties (e.g. drug conjugates and additional binding moieties) and downstream purification processes.

In certain embodiments, the modifications are chemically reactive groups including, but not limited to, reactive thiols (e.g. maleimide based reactive groups), reactive amines (e.g. N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g. HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178.), the entirety of which is hereby incorporated by reference for all it teaches.

### Methods of Treatment

Certain aspects of the present disclosure relate to methods of using the chimeric receptors and genetically modified cells of the present disclosure (e.g., immunoresponsive cells) that express such chimeric receptors to treat subjects in need thereof. In some embodiments, the methods of the present disclosure are useful for treating cancer in a subject, such as solid tumor. In some embodiments, the solid tumor is selected from a colorectal cancer, a pancreatic cancer, a lung cancer, and/or a gastric cancer. In some embodiments, the solid tumor is colorectal cancer. In some embodiments, the colorectal cancer is a colorectal carcinoma. In some embodiments, the solid tumor is a lung cancer. In some embodiments, the lung cancer is a lung adenocarcinoma. Other aspects of the present disclosure relate to use of the chimeric receptors and genetically modified cells of the present disclosure (e.g., immunoresponsive cells) that express such chimeric receptors in methods for treating a pathogen infection or other infectious disease in a subject, such as an immunocompromised human subject. In some embodiments, the methods of the present disclosure may comprise administering genetically modified cells of the present disclosure in an amount effective to achieve the desired effect, including without limitation palliation of an existing condition, prevention of a condition, treatment an existing condition, management of an existing condition, or prevention of recurrence or relapse of a condition. In some embodiments, the effective amount can be provided in one or a series of administrations of the genetically modified cells of the present disclosure (e.g., immunoresponsive cells). In some embodiments, an effective amount can be provided in a bolus or by continuous perfusion.

As disclosed herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific cells (e.g., immunoresponsive cells such as T cells or NK cells), cell doses in the range of about 1×10⁵ to 1×10¹⁰ cells/kg (e.g., about 1×10⁹ cells) are typically infused. Upon administration of the cells into the subject and subsequent differentiation, immunoresponsive cells are induced that are specifically directed against the specific antigen. In some embodiments, induction of immunoresponsive cells can include, without limitation, inactivation of antigen-specific cells such as by deletion or anergy. Inactivation is particularly useful to establish or reestablish tolerance such as in autoimmune disorders. The genetically modified cells can be administered by any method known in the art including, but not limited to, intravenous, subcutaneous, intranodal, intratumoral, intrathecal, intrapleural, intraperitoneal and directly to the thymus.

### Therapeutic Treatment

In some embodiments, the methods of the present disclosure increase an immune response in a subject in need thereof. In some embodiments, the methods of the present disclosure include methods for treating and/or preventing a myeloid disorder in a subject. In some embodiments, the subject is a human. In some embodiments, suitable human subjects for therapy may comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., based on percentage of leukemic cells, by palpation, CAT scan, sonogram, mammogram or X-ray; positive biochemical or histopathologic markers on their own are insufficient to identify this population). In some embodiments, a pharmaceutical composition of the present disclosure is administered to these subjects to elicit an anti-tumor response, with the objective of palliating their condition. In some embodiments, reduction in tumor mass occurs as a result of administration of the pharmaceutical composition, but any clinical improvement will constitute a benefit. In some embodiments, clinical improvement includes decreased risk or rate of progression or reduction in pathological consequences of the tumor. In some embodiments, a second group of suitable human subjects are “adjuvant group” subjects. These subjects are individuals who have had a history of a myeloid disorder, but have been responsive to another mode of therapy. The prior therapy may have included, without limitation, surgical resection, radiotherapy, and/or traditional chemotherapy. As a result, these individuals have no clinically measurable tumor. However, they are suspected of being at risk for progression of the disease, either near the original tumor site, or by metastases. In some embodiments, this group can be further subdivided into high-risk and low-risk individuals. The subdivision can be made on the basis of features observed before or after the initial treatment. These features are known in the clinical arts, and are suitably defined for each different myeloid disorder. Features typical of high-risk subgroups are those in which the tumor has invaded neighboring tissues, or who show involvement of lymph nodes.

In any and all aspects of increasing an immune response as described herein, any increase or decrease or alteration of an aspect of characteristic(s) or function(s) is as compared to a cell not contacted with an immunoresponsive cell as described herein.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response.

In some embodiments, a further group of subjects are those having a genetic predisposition to a malignant disorder, but that have not yet evidenced clinical signs of the malignant disorder. For example, women testing positive for a genetic mutation associated with a malignant disorder, but still of childbearing age, may benefit from receiving one or more of the cells of the present disclosure (e.g., immunoresponsive cells) in treatment prophylactically to prevent the occurrence of malignant disorder until it is suitable to perform chemotherapy, radiation-based therapy, and/or preventive surgery. In some embodiments, the subjects can have an advanced form of disease, in which case the treatment objective can include mitigation or reversal of disease progression, and/or amelioration of side effects. In some embodiments, the subjects may have a history of the condition, for which they have already been treated, in which case the therapeutic objective may typically include a decrease or delay in the risk of recurrence.

### Combination Therapies

In some embodiments, genetically modified cells of the present disclosure (e.g., immunoresponsive ells) expressing one or more chimeric receptors of the present disclosure may be used in combination with other known agents and therapies. In some embodiments, a combination therapy of the present disclosure comprises a genetically modified cells of the present disclosure that can be administered in combination with one or more additional therapeutic agents. In some embodiments, the genetically modified cell and the one or more additional therapeutic agents can be administered simultaneously, in the same or in separate compositions, or sequentially. For sequential administration, the genetically modified can be administered first, and the one or more additional agents can be administered second, or the order of administration can be reversed. In some embodiments, the genetically modified cells are further modified to express one or more additional therapeutic agents.

In some embodiments, a genetically modified cell of the present disclosure may be used in a treatment regimen in combination with surgery, chemotherapy, radiation, immunosuppressive agents (e.g., cyclosporin, azathioprine, methotrexate, mycophenolate, and FK506), antibodies, or other immunoablative agents (e.g., CAMPATH or anti-CD3 antibodies), cytoxin, fludarabme, cyclosporin, FK506, rapamycin, mycophenolic acid, steroids, FR901228, cytokines, irradiation, and peptide vaccines.

In some embodiments, a genetically modified cell of the present disclosure may be used in combination with a lymphodepleting agent. Suitable lymphodepleting agents reduce or decrease lymphocytes, e.g., B cell lymphocytes and/or T cell lymphocytes, prior to immunotherapy. Examples of suitable lymphodepleting agents include, without limitation, fludarabine, cyclophosphamide, corticosteroids, alemtuzumab, total body irradiation (TBI), and any combination thereof.

In some embodiments, a genetically modified cell of the present disclosure may be used in combination with a chemotherapeutic agent. Suitable chemotherapeutic agents include, without limitation, an anthracycline (e.g., doxorubicin), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (e.g., folic acid antagonists, pyrimidine analogs, purine analogs and adenosine deaminase inhibitors, such as fludarabine), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

Examples of general chemotherapeutic agents suitable for use in combination therapies include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Piatinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idaniycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Examples of suitable alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®. Uracilmostaza®, Uramustin®, Uramustine®), chlormethine (Mustargen®), cyclophosphamide (Cytoxan®, Neosar®, Clafen®, Endoxan®, Procytox®, Rev Immune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamme (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Aitretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Examples of suitable mTOR inhibitors include, without limitation, temsirolimus, ridaforolimus (deferolimus), AP23573, MK8669, everolimus (Afimtor® or RADOOl), rapamycin (AY22989, Sirolmius®), and XL765.

Examples of suitable immunomodulators include, without limitation, afutuzumab, pegfilgrastim (Neulasta®), lenalidomide (CC-5013, Revlimid®), thalidomide (Thalomid®), actimid (CC4047), and IRX-2.

Examples of suitable anthracyclines include, without limitation, doxorubicin (Adriamycin® and Rubex®); bleomycin (Lenoxane®); daunorubicin (dauorubicin hydrochloride, daunomyem, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PES®); mitomycin C (Mutamycin®); geldanamycin; herbimycin; ravidomycin; and desacet lravidomycin.

Examples of suitable vinca alkaloids include, without limitation, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbme®).

Examples of suitable proteosome inhibitors include, without limitation, bortezomib (Velcade®); carfilzomib; marizomib (NPI-0052); ixazomib citrate (MLN-9708); delanzomib (CEP-18770); and ONX-0912.

In some embodiments, a genetically modified cell of the present disclosure is administered in combination with a CD20 inhibitor, e.g., an anti-CD20 antibody, or fragment thereof. Exemplary anti-CD20 antibodies include, without limitation, rituximab, ofatumumab, ocrelizumab, veltuzumab, obinutuzumab, TRU-015 (Trubion Pharmaceuticals), ocaratuzumab, and Prol31921.

In some embodiments, a genetically modified cell of the present disclosure is administered in combination with an oncolytic virus. In some embodiments, oncolytic viruses are capable of selectively replicating in and triggering the death of or slowing the growth of a cancer cell. In some cases, oncolytic viruses have no effect or a minimal effect on non-cancer cells. Suitable oncolytic viruses include, without limitation, an oncolytic adenovirus, oncolytic Herpes Simplex Viruses, oncolytic retrovirus, oncolytic parvovirus, oncolytic vaccinia virus, oncolytic Sindbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)). In some embodiments, the oncolytic virus is a recombinant oncolytic virus.

In some embodiments, a genetically modified cell of the present disclosure is administered to a subject in combination with a protein tyrosine phosphatase inhibitor, e.g., a SHP-I inhibitor or a SHP-2 inhibitor. In one embodiment, a genetically modified cell of the present disclosure can be used in combination with a kinase inhibitor. Examples of suitable kinase inhibitors include, without limitation, CDK4 inhibitors, CDK4/6 inhibitors, BTK inhibitors, phosphatidylinositol 3-kinase (PI3K) inhibitors, mTOR inhibitors, MNK inhibitors, and anaplastic lymphoma kinase (ALK) inhibitors.

In some embodiments, a genetically modified cell of the present disclosure is administered to a subject in combination with a modulator of myeloid-derived suppressor cells (MDSCs). MDSCs accumulate in the periphery and at the tumor site of many solid tumors. These cells suppress T cell responses, thereby hindering the efficacy of chimeric receptor-expressing cell therapy. Without being bound by theory, it is believed that administration of a MDSC modulator enhances the efficacy of a genetically modified cell of the present disclosure. Examples of suitable modulators of MDSCs include, without limitation, MCS 110 and BLZ945.

In some embodiments, a genetically modified cell of the present disclosure is administered to a subject in combination with an agent that inhibits or reduces the activity of immunosuppressive plasma cells. Immunosuppressive plasma cells have been shown to impede T cell-dependent immunogenic chemotherapy, such as oxaliplatin (Shalapour et al., Nature 2015, 521:94-101). In one embodiment, immunosuppressive plasma cells can express one or more of IgA, interleukin (IL)-10, and PD-L1.

In some embodiments, a genetically modified cell of the present disclosure is administered to a subject in combination with an interleukin-15 (IL-15) polypeptide, an interleukin-15 receptor alpha (IL-I5Ra) polypeptide, or a combination of both an IL-15 polypeptide and an IL-15Ra polypeptide. In some embodiments, a genetically modified cell of the present disclosure is further modified to express an interleukin-15 (IL-15) polypeptide, an interleukin-15 receptor alpha (IL-I5Ra) polypeptide, or a combination of both an IL-15 polypeptide and an IL-15Ra polypeptide.

In some embodiments, a subject having a malignancy (e.g., a solid tumor) is administered a genetically modified cell of the present disclosure in combination with an agent, e.g., cytotoxic or chemotherapy agent, a biologic therapy (e.g., antibody, e.g., monoclonal antibody, or cellular therapy), or an inhibitor (e.g., kinase inhibitor). In some embodiments, the subject is administered a genetically modified cell of the present disclosure in combination with a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. CPX-351 is a liposomal formulation comprising cytarabine and daunorubicin at a 5: 1 molar ratio. In some embodiments, the subject is administered a chimeric receptor-expressing cell described herein in combination with a hypomethylating agent, e.g., a DNA methyltransferase inhibitor, e.g., azacytidine or decitabine. In some embodiments, the subject is administered a genetically modified cell of the present disclosure in combination with a biologic therapy, e.g., an antibody or cellular therapy, e.g., 225Ac-lintuzumab (Actimab-A; Actinium Pharmaceuticals), IPH2102 (Innate Pharma/Bristol Myers Squibb), SGN-CD33A (Seattle Genetics), or gemtuzumab ozogamicin (Mylotarg; Pfizer). In some embodiments, the subject is administered a genetically modified cell of the present disclosure in combination a FLT3 inhibitor, e.g., sorafenib (Bayer), midostaurin (Novartis), quizartinib (Daiichi Sankyo), crenoianib (Arog Pharmaceuticals), PLX3397 (Daiichi Sankyo), AKN-028 (Akinion Pharmaceuticals), or ASP2215 (Astelias). In some embodiments, the subject is administered a genetically modified cell of the present disclosure in combination with an isocitrate dehydrogenase (IDH) inhibitor, e.g., AG-221 (Celgene/Agios) or AG-120 (Agios/Celgene). In some embodiments, the subject is administered a genetically modified cell of the present disclosure in combination with a cell cycle regulator, e.g., inhibitor of polo-like kinase 1 (Plk1), e.g., volasertib (Boehringer Ingelheim); or an inhibitor of cyclin-dependent kinase 9 (Cdk9), e.g., alvocidib (Tolero Pharmaceuticals/Sanofi Aventis). In some embodiments, the subject is administered a genetically modified cell of the present disclosure in combination with a B cell receptor signaling network inhibitor, e.g., an inhibitor of B-cell lymphoma 2 (Bcl-2), e.g., venetoclax (Abbvie/Roche); or an inhibitor of Button's tyrosine kinase (Btk), e.g., ibrutinib (Pharmacyclics/Johnson & Johnson Janssen Pharmaceutical). In some embodiments, the subject is administered a genetically modified cell of the present disclosure in combination with an inhibitor of M1 aminopeptidase; an inhibitor of histone deacetylase (HDAC), e.g., pracinostat (MEI Pharma); a multi-kinase inhibitor, e.g., rigosertib (Onconova Therapeutics/Baxter/SymBio); or a peptidic CXCR4 inverse agonist, e.g., BL-8040 (BioLineRx).

In some embodiments, a subject can be administered an agent which enhances the activity or fitness of a genetically modified cell of the present disclosure. For example, the agent may inhibit a molecule that modulates or regulates function of an immune cell (e.g., a T cell or NK cell). In some embodiments, the molecule that modulates or regulates immune cell function is an inhibitory molecule. In some embodiments, inhibitory molecules, such as Programmed Death 1 (PD-1) can decrease the ability of the genetically modified cell to mount an immune effector response. Examples of suitable inhibitory molecules include, without limitation, PD-1, PD-L1, CTLA4, TIM3, CEACAM (e.g., CEACAM-1, CEACAM-3 and/or CEACAM-5), LAG3, VISTA, BTLA, TIGIT, LAIR1, CD 160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta. Inhibition of a molecule that modulates or regulates, e.g., inhibits, immune cell function, e.g., by inhibition at the DNA, RNA or protein level, can optimize the performance of genetically modified cells of the present disclosure. In some embodiments, an agent, e.g., an inhibitory polynucleotide, e.g., an inhibitory polynucleotide, e.g., an inhibitory polynucleotide, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), can be used to inhibit expression of an inhibitory molecule in the genetically modified cell. In one embodiment, the inhibitor is an shRNA. In some embodiments, a genetically modified cell of the present disclosure may be further modified to express an inhibitory polynucleotide, e.g., an inhibitory polynucleotide, e.g., an inhibitory polynucleotide, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), can be used to inhibit expression of an inhibitory molecule in the genetically modified cell.

In one embodiment, the agent that modulates or regulates, e.g., inhibits, immune cell function is inhibited within a genetically modified cell of the present disclosure. In such embodiments, a dsRNA molecule that inhibits expression of a molecule that modulates or regulates, e.g., inhibits, immune cell function is linked to the polynucleotide that encodes a component, e.g., all of the components, of a chimeric receptor of the present disclosure. In one embodiment, a polynucleotide molecule that encodes a dsRNA molecule that inhibits expression of the molecule that modulates or regulates, e.g., inhibits, immune cell function is operably linked to a promoter, e.g., a HI- or a U6-derived promoter such that the dsRNA molecule that inhibits expression of the molecule that modulates or regulates, e.g., inhibits, immune cell function is expressed, e.g., is expressed within a the genetically modified cell. In one embodiment the polynucleotide molecule that encodes a dsRNA molecule that inhibits expression of the molecule that modulates or regulates, e.g., inhibits, immune cell function is present on the same vector, e.g., a lentiviral vector, that comprises a polynucleotide molecule that encodes a component, e.g., all of the components, of the chimeric receptor. In such an embodiment the polynucleotide molecule that encodes a dsRNA molecule that inhibits expression of the molecule that modulates or regulates, e.g., inhibits, immune cell function is located on the vector, e.g., the lentiviral vector, 5′- or 3′- to the polynucleotide that encodes a component, e.g., all of the components, of the chimeric receptor. The polynucleotide molecule that encodes a dsRNA molecule that inhibits expression of the molecule that modulates or regulates, e.g., inhibits, immune cell function can be transcribed in the same or different direction as the polynucleotide that encodes a component, e.g., all of the components, of the chimeric receptor. In one embodiment the polynucleotide molecule that encodes a dsRNA molecule that inhibits expression of the molecule that modulates or regulates, e.g., inhibits, immune cell function is present on a vector other than the vector that comprises a polynucleotide molecule that encodes a component, e.g., all of the components, of the chimeric receptor. In one embodiment, the polynucleotide molecule that encodes a dsRNA molecule that inhibits expression of the molecule that modulates or regulates, e.g., inhibits, immune cell function is transiently expressed within the genetically modified cell. In one embodiment, the polynucleotide molecule that encodes a dsRNA molecule that inhibits expression of the molecule that modulates or regulates, e.g., inhibits, immune cell function is stably integrated into the genome of a genetically modified cell of the present disclosure.

In one embodiment, an agent that modulates or regulates, e.g., inhibits, immune cell function can be an antibody or antibody fragment that binds to an inhibitory molecule. For example, the agent can be an antibody or antibody fragment that binds to PD-1, PD-L1, PD-L2 or CTLA4. In one embodiment, the agent is an antibody or antibody fragment that binds to TIM3. In one embodiment, the agent is an antibody or antibody fragment that binds to LAG3.

In some embodiments, the agent which enhances the activity of the genetically modified cell is a CEACAM inhibitor (e.g., CEACAM-1, CEACAM-3, and/or CEACAM-5 inhibitor). In one embodiment, the inhibitor of CEACAM is an anti-CEACAM antibody molecule. In one embodiment, the agent which enhances activity of a genetically modified cell of the present disclosure is miR-17-92. In some embodiments, the agent which enhances the activity of the genetically modified cell is CD40L. In some embodiments, the agent which enhances the activity of the genetically modified cell is GM-CSF. In some embodiments, a genetically modified cell of the present disclosure is further modified to express an antibody or antibody fragment that binds to an inhibitory molecule of the present disclosure.

In one embodiment, the agent which enhances activity of a genetically modified cell of the present disclosure is a cytokine. Cytokines have important functions related to immunoresponsive cell expansion, differentiation, survival, and homeostats. Cytokines that can be administered to the subject receiving a genetically modified cell of the present disclosure include, without limitation, IL-2, IL-4, IL-7, IL-9, IIL-12, L-15, IL-18, and IL-21, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days. In some embodiments, a genetically modified cell of the present disclosure is further modified to express one or more cytokines, such as IL-2, IL-4, IL-7, IL-9, IL-12, L-15, IL-18, and IL-21.

In some embodiments, the cytokine can be administered simultaneously or concurrently with the genetically modified cells, e.g., administered on the same day. The cytokine may be prepared in the same pharmaceutical composition as the genetically modified cells, or may be prepared in a separate pharmaceutical composition. Alternatively, the cytokine can be administered shortly after administration of the genetically modified cells, e.g., 1 day, 2 days, 3 days, 4 days, 5 days, 6 days, or 7 days after administration of the genetically modified cells. In some embodiments where the cytokine is administered in a dosing regimen that occurs over more than one day, the first day of the cytokine dosing regimen can be on the same day as administration with the genetically modified cells, or the first day of the cytokine dosing regimen can be 1 day, 2 days, 3 days, 4 days, 5 days, 6 days, or 7 days after administration of the genetically modified cells. In one embodiment, on the first day, the genetically modified cells are administered to the subject, and on the second day, a cytokine is administered once a day for the next 7 days. In some embodiments, the cytokine is administered for a period of time after administration of the genetically modified cells, e.g., at least 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of the genetically modified cells. In one embodiment, the cytokine is administered after assessment of the subject's response to the genetically modified cells.

### Kits

Certain aspects of the present disclosure relate to kits for the treatment and/or prevention of a cancer (e.g., solid tumors). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing cancer (e.g., a solid tumor). In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### ENUMERATED EMBODIMENTS

1. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region, and
wherein the VH comprises a heavy chain complementarity determining region 3 (CDR-H3) having the amino acid sequence of QGVRPFFDY (SEQ ID NO:4).
2. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region,
wherein the VH comprises a heavy chain complementarity determining region 1 (CDR-H1), a heavy chain complementarity determining region 2 (CDR-H2), and a heavy chain complementarity determining region 3 (CDR-H3), and
wherein the amino acid sequences of CDR-H1, CDR-H2, and CDR-H3 are contained within the VH region amino acid sequence of SEQ ID NO:1.
3. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region, and
wherein the VH comprises:
a heavy chain complementarity determining region 1 (CDR-H1) having the amino acid sequence of GFTFSNS (SEQ ID NO:2),
a heavy chain complementarity determining region 2 (CDR-H2) having the amino acid sequence of SDGGLY (SEQ ID NO:3), and
a heavy chain complementarity determining region 3 (CDR-H3) having the amino acid sequence of QGVRPFFDY (SEQ ID NO:4).
4. The chimeric protein of any one of embodiments 1-3, wherein the VL comprises a light chain complementarity determining region 1 (CDR-L1), a light chain complementarity determining region 2 (CDR-L2), and a light chain complementarity determining region 3 (CDR-L3), wherein the amino acid sequences of CDR-L1, CDR-L2, and CDR-L3 are contained within the VL region amino acid sequence of SEQ ID NO:9 or SEQ ID NO: 10.
5. The chimeric protein of any one of embodiments 1-4, wherein the VL comprises:
a light chain complementarity determining region 1 (CDR-L1) having the amino acid sequence of RASENIYSYLA (SEQ ID NO:11) or RASENLYSYLA (SEQ ID NO:12),
a light chain complementarity determining region 2 (CDR-L2) having the amino acid sequence of NAETLPE (SEQ ID NO:13), and
a light chain complementarity determining region 3 (CDR-L3) having the amino acid sequence of QHHYVIPWT (SEQ ID NO:14).
6. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region,
wherein the VL comprises a light chain complementarity determining region 1 (CDR-L1), a light chain complementarity determining region 2 (CDR-L2), and a light chain complementarity determining region 3 (CDR-L3), and
wherein the amino acid sequences of CDR-L1, CDR-L2, and CDR-L3 are contained within the VL region amino acid sequence of SEQ ID NO:9 or SEQ ID NO: 10.
7. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region, and
wherein the VL comprises:
   a light chain complementarity determining region 1 (CDR-L1) having the amino acid sequence of RASENIYSYLA (SEQ ID NO:11) or RASENLYSYLA (SEQ ID NO:12),
   a light chain complementarity determining region 2 (CDR-L2) having the amino acid sequence of NAETLPE (SEQ ID NO:13), and
   a light chain complementarity determining region 3 (CDR-L3) having the amino acid sequence of QHHYVIPWT (SEQ ID NO:14).
8. The chimeric protein of embodiment 6 or embodiment 7, wherein the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region amino acid sequence of SEQ ID NO:1.
9. The chimeric protein of any one of embodiments 6-8, wherein the VH comprises:
a heavy chain complementarity determining region 1 (CDR-H1) having the amino acid sequence of GFTFSNS (SEQ ID NO:2),
a heavy chain complementarity determining region 2 (CDR-H2) having the amino acid sequence of SDGGLY (SEQ ID NO:3), and
a heavy chain complementarity determining region 3 (CDR-H3) having the amino acid sequence of QGVRPFFDY (SEQ ID NO:4).
10. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region,
wherein the VH comprises:
   a heavy chain complementarity determining region 1 (CDR-H1) having the amino acid sequence of GFTFSNS (SEQ ID NO:2),
   a heavy chain complementarity determining region 2 (CDR-H2) having the amino acid sequence of SDGGLY (SEQ ID NO:3), and
   a heavy chain complementarity determining region 3 (CDR-H3) having the amino acid sequence of QGVRPFFDY (SEQ ID NO:4), and
wherein the VL comprises:
   a light chain complementarity determining region 1 (CDR-L1) having the amino acid sequence of RASENIYSYLA (SEQ ID NO: 11) or RASENLYSYLA (SEQ ID NO:12),
   a light chain complementarity determining region 2 (CDR-L2) having the amino acid sequence of NAETLPE (SEQ ID NO:13), and
   a light chain complementarity determining region 3 (CDR-L3) having the amino acid sequence of QHHYVIPWT (SEQ ID NO:14).
11. The chimeric protein of any one of embodiments 1-10, wherein the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1.
12. The chimeric protein of any one of embodiments 1-11, wherein the VH region comprises the amino acid sequence of SEQ ID NO:1.
13. The chimeric protein of any one of embodiments 1-12, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9.
14. The chimeric protein of any one of embodiments 1-12, wherein the VL region comprises the amino acid sequence of SEQ ID NO:9.
15. The chimeric protein of any one of embodiments 1-12, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:10.
16. The chimeric protein of any one of embodiments 1-12, wherein the VL region comprises the amino acid sequence of SEQ ID NO:10.
17. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region, and
wherein the VH comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1.
18. The chimeric protein of embodiment 17, wherein the VH region comprises the amino acid sequence of SEQ ID NO:1.
19. The chimeric of embodiment 17 or embodiment 18, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO: 10.
20. The chimeric of embodiment 17 or embodiment 18, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9.
21. The chimeric protein of embodiment 17 or embodiment 18, wherein the VL region comprises the amino acid sequence of SEQ ID NO:9.
22. The chimeric of embodiment 17 or embodiment 18, wherein the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:10.
23. The chimeric protein of embodiment 17 or embodiment 18, wherein the VL region comprises the amino acid sequence of SEQ ID NO:10.
24. A chimeric protein comprising an antigen-binding domain specific for V-Set And Immunoglobulin Domain Containing 2 (VSIG2) and a heterologous molecule or moiety,
wherein the antigen-binding domain comprises an antibody or antigen-binding fragment thereof,
wherein the antibody or antigen-binding fragment thereof comprises a heavy chain variable (VH) region and a light chain variable (VL) region, and
wherein the VL comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:9 or SEQ ID NO:10.
25. The chimeric protein of embodiment 24, wherein the VL region comprises the amino acid sequence of SEQ ID NO:9.
26. The chimeric protein of embodiment 24, wherein the VL region comprises the amino acid sequence of SEQ ID NO:10.
27. The chimeric protein of any one of embodiments 24-26, wherein the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO:1.
28. The chimeric protein of any one of embodiments 24-26, wherein the VH region comprises the amino acid sequence of SEQ ID NO:1.
29. A chimeric protein comprising an antigen-binding domain specific for V-Set And Immunoglobulin Domain Containing 2 (VSIG2) and a heterologous molecule or moiety,
wherein the antigen-binding domain competes with a reference antibody or antigen-binding fragment thereof for binding to VSIG2,
wherein the reference antibody or antigen-binding fragment thereof comprises a heavy chain variable (VH) region and a light chain variable (VL) region,
wherein the VH comprises:
   a heavy chain complementarity determining region 1 (CDR-H1) having the amino acid sequence of GFTFSNS (SEQ ID NO:2),
   a heavy chain complementarity determining region 2 (CDR-H2) having the amino acid sequence of SDGGLY (SEQ ID NO:3), and
   a heavy chain complementarity determining region 3 (CDR-H3) having the amino acid sequence of QGVRPFFDY (SEQ ID NO:4), and
wherein the VL comprises:
   a light chain complementarity determining region 1 (CDR-L1) having the amino acid sequence of RASENIYSYLA (SEQ ID NO: 11) or RASENLYSYLA (SEQ ID NO:12),
   a light chain complementarity determining region 2 (CDR-L2) having the amino acid sequence of NAETLPE (SEQ ID NO:13), and
   a light chain complementarity determining region 3 (CDR-L3) having the amino acid sequence of QHHYVIPWT (SEQ ID NO:14).
30. A chimeric protein comprising an antigen-binding domain specific for V-Set And Immunoglobulin Domain Containing 2 (VSIG2) and a heterologous molecule or moiety,
wherein the antigen-binding domain binds essentially the same VSIG2 epitope as a reference antibody or antigen-binding fragment thereof,
wherein the reference antibody or antigen-binding fragment thereof comprises a heavy chain variable (VH) region and a light chain variable (VL) region,
wherein the VH comprises:
   a heavy chain complementarity determining region 1 (CDR-H1) having the amino acid sequence of GFTFSNS (SEQ ID NO:2),
   a heavy chain complementarity determining region 2 (CDR-H2) having the amino acid sequence of SDGGLY (SEQ ID NO:3), and
   a heavy chain complementarity determining region 3 (CDR-H3) having the amino acid sequence of QGVRPFFDY (SEQ ID NO:4), and
wherein the VL comprises:
   a light chain complementarity determining region 1 (CDR-L1) having the amino acid sequence of RASENIYSYLA (SEQ ID NO: 11) or the amino acid sequence of RASENLYSYLA (SEQ ID NO:12),
   a light chain complementarity determining region 2 (CDR-L2) having the amino acid sequence of NAETLPE (SEQ ID NO:13), and
   a light chain complementarity determining region 3 (CDR-L3) having the amino acid sequence of QHHYVIPWT (SEQ ID NO:14).
31. A chimeric protein comprising an antigen-binding domain specific for V-Set And Immunoglobulin Domain Containing 2 (VSIG2) and a heterologous molecule or moiety,
wherein the antigen-binding domain binds an epitope of human VSIG2 that is the same as the VSIG2 epitope bound by a reference antibody or antigen-binding fragment thereof,
wherein the reference antibody or antigen-binding fragment thereof comprises a heavy chain variable (VH) region and a light chain variable (VL) region,
wherein the VH comprises:
   a heavy chain complementarity determining region 1 (CDR-H1) having the amino acid sequence of GFTFSNS (SEQ ID NO:2),
   a heavy chain complementarity determining region 2 (CDR-H2) having the amino acid sequence of SDGGLY (SEQ ID NO:3), and
   a heavy chain complementarity determining region 3 (CDR-H3) having the amino acid sequence of QGVRPFFDY (SEQ ID NO:4), and
wherein the VL comprises:
   a light chain complementarity determining region 1 (CDR-L1) having the amino acid sequence of RASENIYSYLA (SEQ ID NO: 11) or RASENLYSYLA (SEQ ID NO:12),
   a light chain complementarity determining region 2 (CDR-L2) having the amino acid sequence of NAETLPE (SEQ ID NO:13), and
   a light chain complementarity determining region 3 (CDR-L3) having the amino acid sequence of QHHYVIPWT (SEQ ID NO:14).
32. The chimeric protein of any one of embodiments 29-31, wherein the VH region of the reference antibody or antigen-binding fragment thereof comprises the amino acid sequence of SEQ ID NO:1.
33. The chimeric protein of any one of embodiments 29-32, wherein the VL region of the reference antibody or antigen-binding fragment thereof comprises the amino acid sequence of SEQ ID NO:9 or SEQ ID NO: 10.
34. The chimeric protein of any one of embodiments 1-33, wherein the antigen-binding domain comprises a F(ab) fragment, a F(ab′) fragment, or a single chain variable fragment (scFv).
35. The chimeric protein of embodiment 34, wherein the antigen-binding domain comprises a single chain variable fragment (scFv).
36. The chimeric protein of any one of embodiments 1-35, wherein the VH and VL are separated by a peptide linker.
37. The chimeric protein of embodiment 36, wherein the antigen-binding domain comprises the structure VH-L-VL or VL-L-VH, wherein VH is the heavy chain variable domain, L is the peptide linker, and VL is the light chain variable domain.
38. The chimeric protein of embodiment 36 of embodiment 37, wherein the peptide linker comprises an amino acid sequence selected from the group consisting of: SEQ ID Nos:19-35.
39. The chimeric protein of embodiment 35 or embodiment 36, wherein the scFv comprises an amino acid sequence selected from the group consisting of: SEQ ID Nos:69-74.
40. The chimeric protein of any one of embodiments 1-39, wherein the chimeric protein is an antibody-drug conjugate, and wherein the heterologous molecule or moiety comprises a therapeutic agent.
41. The chimeric protein of any one of embodiments 1-39, wherein the chimeric protein is a chimeric antigen receptor (CAR), and wherein the heterologous molecule or moiety comprises a polypeptide selected from the group consisting of: a transmembrane domain, one or more intracellular signaling domains, a hinge domain, a spacer region, one or more peptide linkers, and combinations thereof.
42. The chimeric protein of embodiment 41, wherein the CAR comprises a transmembrane domain.
43. The chimeric protein of embodiment 41 or embodiment 42, wherein the CAR comprises one or more intracellular signaling domains.
44. The chimeric protein of any one of embodiments 41-43, wherein the CAR is an activating CAR comprising one or more intracellular signaling domains that stimulate an immune response.
45. The chimeric protein of any one of embodiments 41-43, wherein the CAR is an inhibitory CAR comprising one or more intracellular inhibitory domains that inhibit an immune response.
46. The chimeric protein of embodiment 45, wherein the intracellular inhibitory domain comprises an enzymatic inhibitory domain.
47. The chimeric protein of embodiment 45, wherein the intracellular inhibitory domain comprises an intracellular inhibitory co-signaling domain.
48. The chimeric protein of any one of embodiments 41-47, wherein the CAR comprises a spacer region between the antigen-binding domain and the transmembrane domain.
49. The chimeric protein of embodiment 48, wherein the spacer region has an amino acid sequence selected from the group consisting of SEQ ID NOs:39-51.
50. A composition comprising the chimeric protein of any one of embodiments 1-49 and a pharmaceutically acceptable carrier, pharmaceutically acceptable excipient, or a combination thereof.
51. An engineered polynucleotide encoding the chimeric protein of any one of embodiments 1-49.
52. An expression vector comprising the engineered polynucleotide of embodiment 51.
53. A composition comprising the engineered polynucleotide of embodiment 51 or the expression vector of embodiment 52, and a pharmaceutically acceptable carrier, pharmaceutically acceptable excipient, or a combination thereof.
54. A method of making an engineered cell, comprising transducing an isolated cell with the engineered polynucleotide of embodiment 51 or the expression vector of embodiment 52.
55. An engineered cell produced by the method of embodiment 54.
56. An isolated cell comprising the engineered polynucleotide of embodiment 51, the expression vector of embodiment 52, or the composition of embodiment 53.
57. A population of engineered cells expressing the engineered polynucleotide of embodiment 51, the expression vector of embodiment 52.
58. An isolated cell comprising the chimeric protein of any one of embodiments 1-49.
59. A population of engineered cells expressing the chimeric protein of any one of embodiments 1-49.
60. The cell or population of cells of any one of embodiments 56-59, wherein the chimeric protein is recombinantly expressed.
61. The cell or population of cells of any one of embodiments 56-60, wherein the chimeric protein is expressed from a vector or a selected locus from the genome of the cell.
62. The cell or population of cells of any one of embodiments 56-61, wherein the cell or population of cells further comprises one or more tumor-targeting chimeric receptors expressed on the cell surface.
63. The cell or population of cells of embodiment 62, wherein each of the one or more tumor-targeting chimeric receptors is a chimeric antigen receptors (CAR) or an engineered T cell receptor.
64. The cell or population of cells of any one of embodiments 56-63, wherein the cell or population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
65. The cell or population of cells of any one of embodiments 56-64, wherein the cell is autologous.
66. The cell or population of cells of any one of embodiments 56-64, wherein the cell is allogeneic.
67. A pharmaceutical composition comprising an effective amount of the cell or population of engineered cells of any one of embodiments 56-66 and a pharmaceutically acceptable carrier, pharmaceutically acceptable excipient, or a combination thereof.
68. A pharmaceutical composition comprising an effective amount of genetically modified cells expressing the chimeric protein of any one of embodiments 1-49 and a pharmaceutically acceptable carrier, pharmaceutically acceptable excipient, or a combination thereof.
69. The pharmaceutical composition of embodiment 67 or embodiment 68, which is for treating and/or preventing a tumor.
70. A method of treating a subject in need thereof, the method comprising administering a therapeutically effective dose of the composition of embodiment 53, or any of the cells of any one of embodiments 55-66, or the composition of any one of embodiments embodiment 67-69.
71. A method of stimulating a cell-mediated immune response to a tumor cell in a subject, the method comprising administering to a subject having a tumor a therapeutically effective dose of the composition of embodiment 53, or any of the cells of any one of embodiments 55-66, or the composition of any one of embodiments 67-69.
72. A method of treating a subject having a tumor, the method comprising administering a therapeutically effective dose of embodiment 53, or any of the cells of any one of embodiments 55-66, or the composition of any one of embodiments 67-69.
73. A kit for treating and/or preventing a tumor, comprising the chimeric protein of any one of embodiments 1-49.
74. The kit of embodiment 73, wherein the kit further comprises written instructions for using the chimeric protein for producing one or more antigen-specific cells for treating and/or preventing a tumor in a subject.
75. A kit for treating and/or preventing a tumor, comprising the cell or population of cells of any one of embodiments 55-66.
76. The kit of embodiment 75, wherein the kit further comprises written instructions for using the cell for treating and/or preventing a tumor in a subject.
77. A kit for treating and/or preventing a tumor, comprising the engineered polynucleotide of embodiment 51.
78. The kit of embodiment 77, wherein the kit further comprises written instructions for using the polynucleotide for producing one or more antigen-specific cells for treating and/or preventing a tumor in a subject.
79. A kit for treating and/or preventing a tumor, comprising the vector of embodiment 52.
80. The kit of embodiment 79, wherein the kit further comprises written instructions for using the vector for producing one or more antigen-specific cells for treating and/or preventing a tumor in a subject.
81. A kit for treating and/or preventing a tumor, comprising the composition of any one of embodiments 53, or 67-69.
82. The kit of embodiment 81, wherein the kit further comprises written instructions for using the composition for treating and/or preventing a tumor in a subject.

### EXAMPLES

The following are examples of methods and compositions of the present disclosure. It is understood that various other embodiments may be practiced, given the general description provided herein.

Below are examples of specific embodiments for carrying out the claimed subject matter of the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1: Anti-VSIG2 Antibody Sequence Determination

### Methods

### Antibody Sequencing

Mouse anti-human VSIG2 monoclonal antibody clone (“Ab”) was sequenced. Briefly, samples containing each of the immunoglobulin chains were digested by various enzymes then analyzed by LC-MS/MS. Peptides were characterized from LC-MS/MS data using de novo peptide sequencing and then assembled into antibody sequences.

The Ab anti-VSIG2 antibody was peptide sequenced. LC-MS/MS data of multiple enzyme digestions were mapped to the assembled antibody sequences. In the heavy chain and light chain, 100% of amino acid residues were covered by at least 5 peptide scans, with significant supporting fragment ions (data not shown).

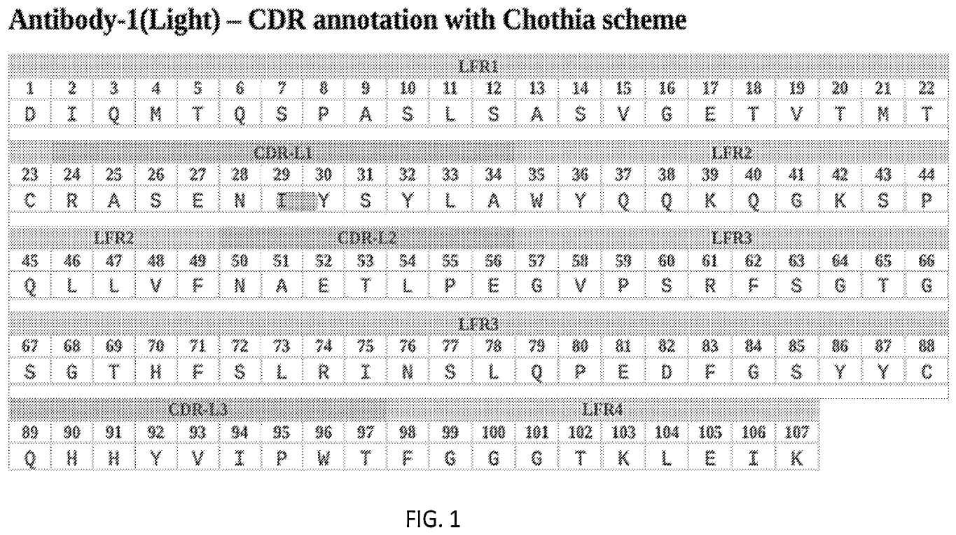

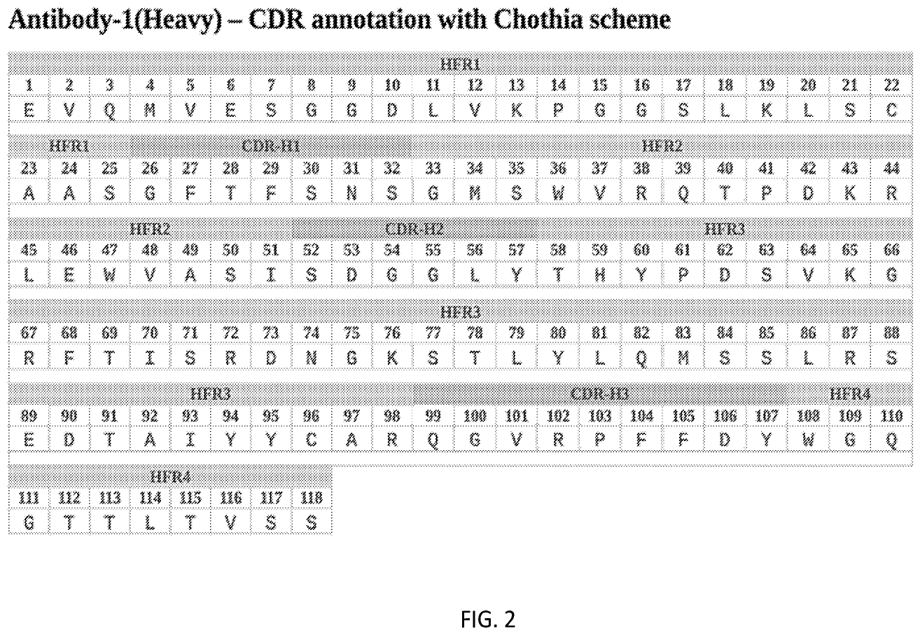

Sequencing results for the light-chain and heavy-chain variable regions are shown in FIG. 1 and FIG. 2, respectively. The framework and complementarity determining regions (CDRs) are annotated according to the Chothia annotation and numbering scheme. Sequences are presented in Table A. Given that Leucine (L) and Isoleucine (I) have the same residue mass, additional analysis techniques were used to identify these residues. The isoleucine residue at position 21 in CDR-L1 as shown in FIG. 1 was determined to be isoleucine but was not confirmed with 100% confidence.

TABLE A
Anti-VSIG2 Antibody (Ab) Sequences

| | SEQ ID | |
|---|---|---|
| Amino Acid Sequence | NO: | Description |
| EVQMVESGGDLVKPGGSLKLSCAASGFTFSNSGMSW | 1 | anti-VSIG2 antibody heavy |
| VRQTPDKRLEWVASISDGGLYTHYPDSVKGRFTISRD | | chain variable (VH) region |
| NGKSTLYLQMSSLRSEDTAIYYCARQGVRPFFDYWG | | |
| QGTTLTVSS | | |
| GFTFSNS | 2 | anti-VSIG2 antibody heavy |
| | | chain complementarity |
| | | determining region 1 |
| SDGGLY | 3 | anti-VSIG2 antibody heavy |
| | | chain complementarity |
| | | determining region 2 |
| QGVRPFFDY | 4 | anti-VSIG2 antibody heavy |
| | | chain complementarity |
| | | determining region 3 |
| EVQMVESGGDLVKPGGSLKLSCAAS | 5 | anti-VSIG2 antibody heavy |
| | | chain framework region 1 |
| GMSWVRQTPDKRLEWVASI | 6 | anti-VSIG2 antibody heavy |
| | | chain framework region 2 |
| THYPDSVKGEDTAIYYCAR | 7 | anti-VSIG2 antibody heavy |
| | | chain framework region 3 |
| WGQGTTLTVSS | 8 | anti-VSIG2 antibody heavy |
| | | chain framework region 4 |
| DIQMTQSPASLSASVGETVTMTCRASENIYSYLAWYQ | 9 | anti-VSIG2 antibody light |
| QKQGKSPQLLVFNAETLPEGVPSRFSGTGSGTHFSLRI | | chain variable (VL) region |
| NSLQPEDFGSYYCQHHYVIPWTFGGGTKLEIK | | version 1 |
| DIQMTQSPASLSASVGETVTMTCRASENLYSYLAWY | 10 | anti-VSIG2 antibody light |
| QQKQGKSPQLLVFNAETLPEGVPSRFSGTGSGTHFSL | | chain variable (VL) region |
| RINSLQPEDFGSYYCQHHYVIPWTFGGGTKLEIK | | version 2 |
| RASENIYSYLA | 11 | anti-VSIG2 antibody light |
| | | chain complementarity |
| | | determining region 1 |
| | | version 1 |
| RASENLYSYLA | 12 | anti-VSIG2 antibody light |
| | | chain complementarity |
| | | determining region 1 |
| | | version 2 |
| NAETLPE | 13 | anti-VSIG2 antibody light |
| | | chain complementarity |
| | | determining region 2 |
| QHHYVIPWT | 14 | anti-VSIG2 antibody light |
| | | chain complementarity |
| | | determining region 3 |
| DIQMTQSPASLSASVGETVTMTC | 15 | anti-VSIG2 antibody light |
| | | chain framework region 1 |
| WYQQKQGKSPQLLVF | 16 | anti-VSIG2 antibody light |
| | | chain framework region 2 |
| GVPSRFSGTGSGTHFSLRINSLQPEDFGSYYC | 17 | anti-VSIG2 antibody light |
| | | chain framework region 3 |
| FGGGTKLEIK | 18 | anti-VSIG2 antibody light |
| | | chain framework region 4 |

### Example 2: Anti-VSIG2 Activating CAR Production & Evaluation

CAR constructs were cloned into a lentiviral vector. Lentivirus is produced using the Lenti-X 293T system. The antigen specificity and domain organization for the CAR constructs examined are described in Table B below, and scFv amino acid sequences and nucleotide sequences are presented in Table C and Table D, respectively.

TABLE B
Activating CAR Constructs (Activating)

| Construct | | | SEQ |
|---|---|---|---|
| (s) | Description | Sequence | ID NO |
| SB04741/ | CD8ss-VSIG2 | MALPVTALLLPLALLLHAARPEVQMVESGGDLVKPGGSLKLSC | 63 |
| SB04747 | Ab scFv (VH- | AASGFTFSNSGMSWVRQTPDKRLEWVASISDGGLYTHYPDSVK | |
| | (G4S)3-VL)- | GRFTISRDNGKSTLYLQMSSLRSEDTAIYYCARQGVRPFFDYWG | |
| | V5 tag-CD8 | QGTTLTVSSGGGGSGGGGSGGGGSDIQMTQSPASLSASVGETVT | |
| | hinge/CD28 | MTCRASENIYSYLAWYQQKQGKSPQLLVFNAETLPEGVPSRFSG | |
| | TM/CD28 | TGSGTHFSLRINSLQPEDFGSYYCQHHYVIPWTFGGGTKLEIKGK | |
| | ICD-CD3z | PIPNPLLGLDSTNGAATTTPAPRPPTPAPTIALQPLSLRPEACRPA | |
| | (G4S)3 is | AGGAVHTRGLDFACDFWVLVVVGGVLACYSLLVTVAFIIFWVR | |
| | SEQ ID NO: | SKRSRLLHSDYMNMTPRRPGPTRKHYQPYAPPRDFAAYRSRVK | |
| | 31 | FSRSADAPAYKQGQNQLYNELNLGRREEYDVLDKRRGRDPEM | |
| | | GGKPRRKNPQEGLYNELQKDKMAEAYSEIGMKGERRRGKGHD | |
| | | GLYQGLSTATKDTYDALHMQALPPR | |
| SB04742/ | CD8ss-VSIG2 | MALPVTALLLPLALLLHAARPDIQMTQSPASLSASVGETVTMTC | 64 |
| SB04748 | Ab scFv (VL- | RASENIYSYLAWYQQKQGKSPQLLVFNAETLPEGVPSRFSGTGS | |
| | (G4S)3-VH)- | GTHFSLRINSLQPEDFGSYYCQHHYVIPWTFGGGTKLEIKGGGG | |
| | V5 tag-CD8 | SGGGGSGGGGSEVQMVESGGDLVKPGGSLKLSCAASGFTFSNS | |
| | hinge/ CD28 | GMSWVRQTPDKRLEWVASISDGGLYTHYPDSVKGRFTISRDNG | |
| | TM/CD28 | KSTLYLQMSSLRSEDTAIYYCARQGVRPFFDYWGQGTTLTVSSG | |
| | ICD-CD3z | KPIPNPLLGLDSTNGAATTTPAPRPPTPAPTIALQPLSLRPEACRP | |
| | (G4S)3 is | AAGGAVHTRGLDFACDFWVLVVVGGVLACYSLLVTVAFIIFWV | |
| | SEQ ID NO: | RSKRSRLLHSDYMNMTPRRPGPTRKHYQPYAPPRDFAAYRSRV | |
| | 31 | KFSRSADAPAYKQGQNQLYNELNLGRREEYDVLDKRRGRDPE | |
| | | MGGKPRRKNPQEGLYNELQKDKMAEAYSEIGMKGERRRGKGH | |
| | | DGLYQGLSTATKDTYDALHMQALPPR | |
| SB04743/ | CD8ss-VSIG2 | MALPVTALLLPLALLLHAARPEVQMVESGGDLVKPGGSLKLSC | 65 |
| SB04749 | Ab scFv (VH- | AASGFTFSNSGMSWVRQTPDKRLEWVASISDGGLYTHYPDSVK | |
| | (Whitlow)- | GRFTISRDNGKSTLYLQMSSLRSEDTAIYYCARQGVRPFFDYWG | |
| | VL)-V5 tag- | QGTTLTVSSGSTSGSGKPGSGEGSTKGDIQMTQSPASLSASVGET | |
| | CD8 hinge/ | VTMTCRASENIYSYLAWYQQKQGKSPQLLVFNAETLPEGVPSRF | |
| | CD28 TM/ | SGTGSGTHFSLRINSLQPEDFGSYYCQHHYVIPWTFGGGTKLEIK | |
| | CD28 ICD- | GKPIPNPLLGLDSTNGAATTTPAPRPPTPAPTIALQPLSLRPEACR | |
| | CD3z | PAAGGAVHTRGLDFACDFWVLVVVGGVLACYSLLVTVAFIIFW | |
| | | VRSKRSRLLHSDYMNMTPRRPGPTRKHYQPYAPPRDFAAYRSR | |
| | | VKFSRSADAPAYKQGQNQLYNELNLGRREEYDVLDKRRGRDPE | |
| | | MGGKPRRKNPQEGLYNELQKDKMAEAYSEIGMKGERRRGKGH | |
| | | DGLYQGLSTATKDTYDALHMQALPPR | |
| SB04744/ | CD8ss-VSIG2 | MALPVTALLLPLALLLHAARPDIQMTQSPASLSASVGETVTMTC | 66 |
| SB04750 | Ab scFv (VL- | RASENIYSYLAWYQQKQGKSPQLLVFNAETLPEGVPSRFSGTGS | |
| | (Whitlow)- | GTHFSLRINSLQPEDFGSYYCQHHYVIPWTFGGGTKLEIKGSTSG | |
| | VH)-V5 tag- | SGKPGSGEGSTKGEVQMVESGGDLVKPGGSLKLSCAASGFTFSN | |
| | CD8 hinge/ | SGMSWVRQTPDKRLEWVASISDGGLYTHYPDSVKGRFTISRDN | |
| | CD28 TM/ | GKSTLYLQMSSLRSEDTAIYYCARQGVRPFFDYWGQGTTLTVSS | |
| | CD28 ICD- | GKPIPNPLLGLDSTNGAATTTPAPRPPTPAPTIALQPLSLRPEACR | |
| | CD3z | PAAGGAVHTRGLDFACDFWVLVVVGGVLACYSLLVTVAFIIFW | |
| | | VRSKRSRLLHSDYMNMTPRRPGPTRKHYQPYAPPRDFAAYRSR | |
| | | VKFSRSADAPAYKQGQNQLYNELNLGRREEYDVLDKRRGRDPE | |
| | | MGGKPRRKNPQEGLYNELQKDKMAEAYSEIGMKGERRRGKGH | |
| | | DGLYQGLSTATKDTYDALHMQALPPR | |
| SB04745 | CD8ss-VSIG2 | MALPVTALLLPLALLLHAARPEVQMVESGGDLVKPGGSLKLSC | 67 |
| | Ab scFv (VH- | AASGFTFSNSGMSWVRQTPDKRLEWVASISDGGLYTHYPDSVK | |
| | (G4S)-VL)- | GRFTISRDNGKSTLYLQMSSLRSEDTAIYYCARQGVRPFFDYWG | |
| | V5 tag-CD8 | QGTTLTVSSGGGGSDIQMTQSPASLSASVGETVTMTCRASENIY | |
| | hinge/CD28 | SYLAWYQQKQGKSPQLLVFNAETLPEGVPSRFSGTGSGTHFSLR | |
| | TM/CD28 | INSLQPEDFGSYYCQHHYVIPWTFGGGTKLEIKGKPIPNPLLGLD | |
| | ICD-CD3z | STNGAATTTPAPRPPTPAPTIALQPLSLRPEACRPAAGGAVHTRG | |
| | (G4S) is SEQ | LDFACDFWVLVVVGGVLACYSLLVTVAFIIFWVRSKRSRLLHSD | |
| | ID NO: 29 | YMNMTPRRPGPTRKHYQPYAPPRDFAAYRSRVKFSRSADAPAY | |
| | | KQGQNQLYNELNLGRREEYDVLDKRRGRDPEMGGKPRRKNPQ | |
| | | EGLYNELQKDKMAEAYSEIGMKGERRRGKGHDGLYQGLSTAT | |
| | | KDTYDALHMQALPPR | |
| SB04746 | CD8ss-VSIG2 | MALPVTALLLPLALLLHAARPDIQMTQSPASLSASVGETVTMTC | 68 |
| | Ab scFv (VL- | RASENIYSYLAWYQQKQGKSPQLLVFNAETLPEGVPSRFSGTGS | |
| | (G4S)-VH)- | GTHFSLRINSLQPEDFGSYYCQHHYVIPWTFGGGTKLEIKGGGG | |
| | V5 tag-CD8 | SEVQMVESGGDLVKPGGSLKLSCAASGFTFSNSGMSWVRQTPD | |
| | hinge/CD28 | KRLEWVASISDGGLYTHYPDSVKGRFTISRDNGKSTLYLQMSSL | |
| | TM/CD28 | RSEDTAIYYCARQGVRPFFDYWGQGTTLTVSSGKPIPNPLLGLD | |
| | ICD-CD3z | STNGAATTTPAPRPPTPAPTIALQPLSLRPEACRPAAGGAVHTRG | |
| | (G4S) is SEQ | LDFACDFWVLVVVGGVLACYSLLVTVAFIIFWVRSKRSRLLHSD | |
| | ID NO: 29 | YMNMTPRRPGPTRKHYQPYAPPRDFAAYRSRVKFSRSADAPAY | |
| | | KQGQNQLYNELNLGRREEYDVLDKRRGRDPEMGGKPRRKNPQ | |
| | | EGLYNELQKDKMAEAYSEIGMKGERRRGKGHDGLYQGLSTAT | |
| | | KDTYDALHMQALPPR | |

TABLE C
CAR scFv Amino Acid Sequences

| | | SEQ ID |
|---|---|---|
| scFv | Amino Acid Sequence | NO: |
| VSIG2 Ab | EVQMVESGGDLVKPGGSLKLSCAASGFTFSNSGMSWVRQTPDKRLE | 69 |
| scFv (VH- | WVASISDGGLYTHYPDSVKGRFTISRDNGKSTLYLQMSSLRSEDTAIY | |
| (G4S)3-VL) | YCARQGVRPFFDYWGQGTTLTVSSGGGGSGGGGSGGGGSDIQMTQS | |
| (G4S)3 is | PASLSASVGETVTMTCRASENIYSYLAWYQQKQGKSPQLLVFNAETL | |
| SEQ ID | PEGVPSRFSGTGSGTHFSLRINSLQPEDFGSYYCQHHYVIPWTFGGGT | |
| NO: 31 | KLEIK | |
| VSIG2 Ab | DIQMTQSPASLSASVGETVTMTCRASENIYSYLAWYQQKQGKSPQLL | 70 |
| scFv (VL- | VFNAETLPEGVPSRFSGTGSGTHFSLRINSLQPEDFGSYYCQHHYVIP | |
| (G4S)3-VH) | WTFGGGTKLEIKGGGGSGGGGSGGGGSEVQMVESGGDLVKPGGSL | |
| (G4S)3 is | KLSCAASGFTFSNSGMSWVRQTPDKRLEWVASISDGGLYTHYPDSV | |
| SEQ ID | KGRFTISRDNGKSTLYLQMSSLRSEDTAIYYCARQGVRPFFDYWGQG | |
| NO: 31 | TTLTVSS | |
| VSIG2 Ab | EVQMVESGGDLVKPGGSLKLSCAASGFTFSNSGMSWVRQTPDKRLE | 71 |
| scFv (VH- | WVASISDGGLYTHYPDSVKGRFTISRDNGKSTLYLQMSSLRSEDTAIY | |
| (Whitlow)- | YCARQGVRPFFDYWGQGTTLTVSSGSTSGSGKPGSGEGSTKGDIQMT | |
| VL) | QSPASLSASVGETVTMTCRASENIYSYLAWYQQKQGKSPQLLVFNAE | |
| | TLPEGVPSRFSGTGSGTHFSLRINSLQPEDFGSYYCQHHYVIPWTFGG | |
| | GTKLEIK | |
| VSIG2 Ab | DIQMTQSPASLSASVGETVTMTCRASENIYSYLAWYQQKQGKSPQLL | 72 |
| scFv (VL- | VFNAETLPEGVPSRFSGTGSGTHFSLRINSLQPEDFGSYYCQHHYVIP | |
| (Whitlow)- | WTFGGGTKLEIKGSTSGSGKPGSGEGSTKGEVQMVESGGDLVKPGG | |
| VH) | SLKLSCAASGFTFSNSGMSWVRQTPDKRLEWVASISDGGLYTHYPDS | |
| | VKGRFTISRDNGKSTLYLQMSSLRSEDTAIYYCARQGVRPFFDYWGQ | |
| | GTTLTVSS | |
| VSIG2 Ab | EVQMVESGGDLVKPGGSLKLSCAASGFTFSNSGMSWVRQTPDKRLE | 73 |
| scFv (VH- | WVASISDGGLYTHYPDSVKGRFTISRDNGKSTLYLQMSSLRSEDTAIY | |
| (G4S)-VL) | YCARQGVRPFFDYWGQGTTLTVSSGGGGSDIQMTQSPASLSASVGET | |
| (G4S) is | VTMTCRASENIYSYLAWYQQKQGKSPQLLVFNAETLPEGVPSRFSGT | |
| SEQ ID | GSGTHFSLRINSLQPEDFGSYYCQHHYVIPWTFGGGTKLEIK | |
| NO: 29 | | |
| VSIG2 Ab | DIQMTQSPASLSASVGETVTMTCRASENIYSYLAWYQQKQGKSPQLL | 74 |
| scFv (VL- | VFNAETLPEGVPSRFSGTGSGTHFSLRINSLQPEDFGSYYCQHHYVIP | |
| (G4S)-VH) | WTFGGGTKLEIKGGGGSEVQMVESGGDLVKPGGSLKLSCAASGFTFS | |
| (G4S) is | NSGMSWVRQTPDKRLEWVASISDGGLYTHYPDSVKGRFTISRDNGK | |
| SEQ ID | STLYLQMSSLRSEDTAIYYCARQGVRPFFDYWGQGTTLTVSS | |
| NO: 29 | | |

TABLE D
CAR scFv Nucleotide Sequences

| | | SEQ |
|---|---|---|
| scFv | Nucleotide Sequence | ID NO: |
| VSIG2 Ab | GAGGTGCAGATGGTTGAGTCTGGCGGCGATCTGGTTAAGCCTGGC | 75 |
| scFv (VH- | GGAAGCCTGAAGCTGTCTTGTGCCGCCAGCGGCTTCACCTTCAGC | |
| (G4S)3-VL) | AATAGCGGCATGAGCTGGGTCCGACAGACCCCTGACAAGAGACT | |
| DNA Seq 1 | GGAATGGGTCGCCAGCATCTCTGACGGCGGCCTGTACACACACTA | |
| (SB04741) | CCCCGATTCTGTGAAGGGCAGATTCACCATCAGCAGAGACAACGG | |
| (G4S)3 is | CAAGAGCACCCTGTACCTGCAGATGAGCAGCCTGAGAAGCGAGG | |
| SEQ ID NO: | ACACCGCCATCTACTACTGCGCCAGACAGGGCGTCAGACCCTTCT | |
| 31 | TCGATTATTGGGGCCAGGGCACCACACTGACCGTGTCATCTGGCG | |
| | GAGGCGGATCAGGTGGCGGAGGAAGTGGCGGCGGAGGATCTGAC | |
| | ATCCAGATGACACAGTCTCCAGCCAGCCTGTCTGCCTCTGTGGGA | |
| | GAGACAGTGACCATGACCTGTCGGGCCAGCGAGAACATCTACAGC | |
| | TACCTGGCCTGGTATCAGCAGAAGCAGGGCAAGTCTCCTCAGCTG | |
| | CTGGTGTTCAACGCCGAGACACTGCCTGAAGGCGTGCCCAGCAGA | |
| | TTTTCTGGAACAGGCAGCGGCACCCACTTCAGCCTGAGAATCAAT | |
| | AGCCTGCAGCCTGAGGACTTCGGCAGCTACTACTGCCAGCACCAC | |
| | TACGTGATCCCTTGGACCTTTGGCGGAGGCACCAAGCTGGAAATC | |
| | AAG | |
| VSIG2 Ab | GACATCCAGATGACACAGTCTCCAGCCAGCCTGTCTGCCTCTGTG | 76 |
| scFv (VL- | GGAGAGACAGTGACCATGACCTGTCGGGCCAGCGAGAACATCTA | |
| (G4S)3-VH) | CAGCTACCTGGCCTGGTATCAGCAGAAGCAGGGCAAGTCTCCTCA | |
| DNA Seq 1 | GCTGCTGGTGTTCAACGCCGAGACACTGCCTGAAGGCGTGCCCAG | |
| (SB04742) | CAGATTTTCTGGAACAGGCAGCGGCACCCACTTCAGCCTGAGAAT | |
| (G4S)3 is | CAATAGCCTGCAGCCTGAGGACTTCGGCAGCTACTACTGCCAGCA | |
| SEQ ID NO: | CCACTACGTGATCCCTTGGACCTTTGGCGGAGGCACCAAGCTGGA | |
| 31 | AATCAAGGGCGGAGGCGGATCAGGTGGCGGAGGAAGTGGCGGCG | |
| | GAGGATCTGAGGTGCAGATGGTTGAGTCTGGCGGCGATCTGGTTA | |
| | AGCCTGGCGGAAGCCTGAAGCTGTCTTGTGCCGCCAGCGGCTTCA | |
| | CCTTCAGCAATAGCGGCATGAGCTGGGTCCGACAGACCCCTGACA | |
| | AGAGACTGGAATGGGTCGCCAGCATCTCTGACGGCGGCCTGTACA | |
| | CACACTACCCCGATTCTGTGAAGGGCAGATTCACCATCAGCAGAG | |
| | ACAACGGCAAGAGCACCCTGTACCTGCAGATGAGCAGCCTGAGA | |
| | AGCGAGGACACCGCCATCTACTACTGCGCCAGACAGGGCGTCAGA | |
| | CCCTTCTTCGATTATTGGGGCCAGGGCACCACACTGACCGTGTCAT | |
| | CT | |
| VSIG2 Ab | GAGGTGCAGATGGTTGAGTCTGGCGGCGATCTGGTTAAGCCTGGC | 77 |
| scFv (VH- | GGAAGCCTGAAGCTGTCTTGTGCCGCCAGCGGCTTCACCTTCAGC | |
| (Whitlow)- | AATAGCGGCATGAGCTGGGTCCGACAGACCCCTGACAAGAGACT | |
| VL) | GGAATGGGTCGCCAGCATCTCTGACGGCGGCCTGTACACACACTA | |
| DNA Seq 1 | CCCCGATTCTGTGAAGGGCAGATTCACCATCAGCAGAGACAACGG | |
| (SB04743) | CAAGAGCACCCTGTACCTGCAGATGAGCAGCCTGAGAAGCGAGG | |
| | ACACCGCCATCTACTACTGCGCCAGACAGGGCGTCAGACCCTTCT | |
| | TCGATTATTGGGGCCAGGGCACCACACTGACCGTGTCATCTGGCA | |
| | GCACAAGCGGCTCTGGAAAACCTGGATCTGGCGAGGGCTCTACCA | |
| | AGGGCGACATCCAGATGACACAGTCTCCAGCCAGCCTGTCTGCCT | |
| | CTGTGGGAGAGACAGTGACCATGACCTGTCGGGCCAGCGAGAAC | |
| | ATCTACAGCTACCTGGCCTGGTATCAGCAGAAGCAGGGCAAGTCT | |
| | CCTCAGCTGCTGGTGTTCAACGCCGAGACACTGCCTGAAGGCGTG | |
| | CCCAGCAGATTTTCTGGAACAGGCAGCGGCACCCACTTCAGCCTG | |
| | AGAATCAATAGCCTGCAGCCTGAGGACTTCGGCAGCTACTACTGC | |
| | CAGCACCACTACGTGATCCCTTGGACCTTTGGCGGAGGCACCAAG | |
| | CTGGAAATCAAG | |
| VSIG2 Ab | GACATCCAGATGACACAGTCTCCAGCCAGCCTGTCTGCCTCTGTG | 78 |
| scFv (VL- | GGAGAGACAGTGACCATGACCTGTCGGGCCAGCGAGAACATCTA | |
| (Whitlow)- | CAGCTACCTGGCCTGGTATCAGCAGAAGCAGGGCAAGTCTCCTCA | |
| VH) | GCTGCTGGTGTTCAACGCCGAGACACTGCCTGAAGGCGTGCCCAG | |
| DNA Seq 1 | CAGATTTTCTGGAACAGGCAGCGGCACCCACTTCAGCCTGAGAAT | |
| (SB04744) | CAATAGCCTGCAGCCTGAGGACTTCGGCAGCTACTACTGCCAGCA | |
| | CCACTACGTGATCCCTTGGACCTTTGGCGGAGGCACCAAGCTGGA | |
| | AATCAAGGGCAGCACAAGCGGCTCTGGAAAACCTGGATCTGGCG | |
| | AGGGCTCTACCAAGGGCGAGGTGCAGATGGTTGAGTCTGGCGGCG | |
| | ATCTGGTTAAGCCTGGCGGAAGCCTGAAGCTGTCTTGTGCCGCCA | |
| | GCGGCTTCACCTTCAGCAATAGCGGCATGAGCTGGGTCCGACAGA | |
| | CCCCTGACAAGAGACTGGAATGGGTCGCCAGCATCTCTGACGGCG | |
| | GCCTGTACACACACTACCCCGATTCTGTGAAGGGCAGATTCACCA | |
| | TCAGCAGAGACAACGGCAAGAGCACCCTGTACCTGCAGATGAGC | |
| | AGCCTGAGAAGCGAGGACACCGCCATCTACTACTGCGCCAGACAG | |
| | GGCGTCAGACCCTTCTTCGATTATTGGGGCCAGGGCACCACACTG | |
| | ACCGTGTCATCT | |
| VSIG2 Ab | GAGGTGCAGATGGTTGAGTCTGGCGGCGATCTGGTTAAGCCTGGC | 79 |
| scFv (VH- | GGAAGCCTGAAGCTGTCTTGTGCCGCCAGCGGCTTCACCTTCAGC | |
| (G4S)-VL) | AATAGCGGCATGAGCTGGGTCCGACAGACCCCTGACAAGAGACT | |
| (SB04745) | GGAATGGGTCGCCAGCATCTCTGACGGCGGCCTGTACACACACTA | |
| (G4S) is | CCCCGATTCTGTGAAGGGCAGATTCACCATCAGCAGAGACAACGG | |
| SEQ ID NO: | CAAGAGCACCCTGTACCTGCAGATGAGCAGCCTGAGAAGCGAGG | |
| 29 | ACACCGCCATCTACTACTGCGCCAGACAGGGCGTCAGACCCTTCT | |
| | TCGATTATTGGGGCCAGGGCACCACACTGACCGTGTCATCTGGTG | |
| | GCGGAGGCAGCGACATCCAGATGACACAGTCTCCAGCCAGCCTGT | |
| | CTGCCTCTGTGGGAGAGACAGTGACCATGACCTGTCGGGCCAGCG | |
| | AGAACATCTACAGCTACCTGGCCTGGTATCAGCAGAAGCAGGGCA | |
| | AGTCTCCTCAGCTGCTGGTGTTCAACGCCGAGACACTGCCTGAAG | |
| | GCGTGCCCAGCAGATTTTCTGGAACAGGCAGCGGCACCCACTTCA | |
| | GCCTGAGAATCAATAGCCTGCAGCCTGAGGACTTCGGCAGCTACT | |
| | ACTGCCAGCACCACTACGTGATCCCTTGGACCTTTGGCGGAGGCA | |
| | CCAAGCTGGAAATCAAG | |
| VSIG2 Ab | GACATCCAGATGACACAGTCTCCAGCCAGCCTGTCTGCCTCTGTG | 80 |
| scFv (VL- | GGAGAGACAGTGACCATGACCTGTCGGGCCAGCGAGAACATCTA | |
| (G4S)-VH) | CAGCTACCTGGCCTGGTATCAGCAGAAGCAGGGCAAGTCTCCTCA | |
| (SB04746) | GCTGCTGGTGTTCAACGCCGAGACACTGCCTGAAGGCGTGCCCAG | |
| (G4S) is | CAGATTTTCTGGAACAGGCAGCGGCACCCACTTCAGCCTGAGAAT | |
| SEQ ID NO: | CAATAGCCTGCAGCCTGAGGACTTCGGCAGCTACTACTGCCAGCA | |
| 29 | CCACTACGTGATCCCTTGGACCTTTGGCGGAGGCACCAAGCTGGA | |
| | AATCAAGGGTGGCGGAGGCAGCGAGGTGCAGATGGTTGAGTCTG | |
| | GCGGCGATCTGGTTAAGCCTGGCGGAAGCCTGAAGCTGTCTTGTG | |
| | CCGCCAGCGGCTTCACCTTCAGCAATAGCGGCATGAGCTGGGTCC | |
| | GACAGACCCCTGACAAGAGACTGGAATGGGTCGCCAGCATCTCTG | |
| | ACGGCGGCCTGTACACACACTACCCCGATTCTGTGAAGGGCAGAT | |
| | TCACCATCAGCAGAGACAACGGCAAGAGCACCCTGTACCTGCAGA | |
| | TGAGCAGCCTGAGAAGCGAGGACACCGCCATCTACTACTGCGCCA | |
| | GACAGGGCGTCAGACCCTTCTTCGATTATTGGGGCCAGGGCACCA | |
| | CACTGACCGTGTCATCT | |
| VSIG2 Ab | GAGGTGCAGATGGTGGAAAGCGGCGGAGATCTGGTGAAGCCTGG | 81 |
| scFv (VH- | CGGCAGCCTGAAGCTGTCTTGTGCCGCTTCTGGCTTTACCTTCAGC | |
| (G4S)3-VL) | AACAGCGGCATGAGCTGGGTCAGACAGACCCCTGATAAGCGGCT | |
| DNA Seq 2 | GGAATGGGTGGCCAGCATCAGCGACGGCGGACTGTACACCCACTA | |
| (SB04747) | CCCAGACAGCGTGAAAGGCAGATTCACAATCAGCCGGGACAACG | |
| (G4S)3 is | GCAAGTCCACCCTGTATCTGCAGATGTCTAGCCTGAGAAGCGAGG | |
| SEQ ID NO: | ACACCGCCATCTACTACTGCGCCAGACAAGGCGTGCGCCCCTTCT | |
| 31 | TCGACTACTGGGGCCAGGGAACAACACTCACCGTGTCCTCCGGCG | |
| | GAGGCGGATCAGGTGGCGGAGGAAGTGGCGGCGGAGGATCTGAT | |
| | ATCCAGATGACCCAGAGCCCTGCTTCTCTGAGCGCCAGCGTGGGC | |
| | GAGACAGTGACCATGACCTGTAGAGCCTCCGAGAATATCTACAGC | |
| | TACCTGGCCTGGTACCAGCAGAAACAGGGCAAGAGCCCCCAGCTG | |
| | CTGGTCTTTAACGCCGAGACACTGCCTGAAGGCGTGCCATCTAGA | |
| | TTCTCCGGCACCGGCAGCGGCACACACTTCAGCCTCCGGATCAAC | |
| | AGCCTGCAACCTGAGGACTTCGGATCTTATTACTGCCAGCACCAC | |
| | TACGTGATCCCCTGGACCTTCGGCGGAGGCACCAAGCTGGAAATC | |
| | AAG | |
| VSIG2 Ab | GATATCCAGATGACCCAGAGCCCTGCTTCTCTGAGCGCCAGCGTG | 82 |
| scFv (VL- | GGCGAGACAGTGACCATGACCTGTAGAGCCTCCGAGAATATCTAC | |
| (G4S)3-VH) | AGCTACCTGGCCTGGTACCAGCAGAAACAGGGCAAGAGCCCCCA | |
| DNA Seq 2 | GCTGCTGGTCTTTAACGCCGAGACACTGCCTGAAGGCGTGCCATC | |
| SB04748 | TAGATTCTCCGGCACCGGCAGCGGCACACACTTCAGCCTCCGGAT | |
| (G4S)3 is | CAACAGCCTGCAACCTGAGGACTTCGGATCTTATTACTGCCAGCA | |
| SEQ ID NO: | CCACTACGTGATCCCCTGGACCTTCGGCGGAGGCACCAAGCTGGA | |
| 31 | AATCAAGGGCGGAGGCGGATCAGGTGGCGGAGGAAGTGGCGGCG | |
| | GAGGATCTGAGGTGCAGATGGTGGAAAGCGGCGGAGATCTGGTG | |
| | AAGCCTGGCGGCAGCCTGAAGCTGTCTTGTGCCGCTTCTGGCTTTA | |
| | CCTTCAGCAACAGCGGCATGAGCTGGGTCAGACAGACCCCTGATA | |
| | AGCGGCTGGAATGGGTGGCCAGCATCAGCGACGGCGGACTGTAC | |
| | ACCCACTACCCAGACAGCGTGAAAGGCAGATTCACAATCAGCCGG | |
| | GACAACGGCAAGTCCACCCTGTATCTGCAGATGTCTAGCCTGAGA | |
| | AGCGAGGACACCGCCATCTACTACTGCGCCAGACAAGGCGTGCGC | |
| | CCCTTCTTCGACTACTGGGGCCAGGGAACAACACTCACCGTGTCC | |
| | TCC | |
| VSIG2 Ab | GAGGTGCAGATGGTGGAAAGCGGCGGAGATCTGGTGAAGCCTGG | 83 |
| scFv (VH- | CGGCAGCCTGAAGCTGTCTTGTGCCGCTTCTGGCTTTACCTTCAGC | |
| (Whitlow)- | AACAGCGGCATGAGCTGGGTCAGACAGACCCCTGATAAGCGGCT | |
| VL) | GGAATGGGTGGCCAGCATCAGCGACGGCGGACTGTACACCCACTA | |
| DNA Seq 2 | CCCAGACAGCGTGAAAGGCAGATTCACAATCAGCCGGGACAACG | |
| (SB04749) | GCAAGTCCACCCTGTATCTGCAGATGTCTAGCCTGAGAAGCGAGG | |
| | ACACCGCCATCTACTACTGCGCCAGACAAGGCGTGCGCCCCTTCT | |
| | TCGACTACTGGGGCCAGGGAACAACACTCACCGTGTCCTCCGGCA | |
| | GCACAAGCGGCTCTGGAAAACCTGGATCTGGCGAGGGCTCTACCA | |
| | AGGGCGATATCCAGATGACCCAGAGCCCTGCTTCTCTGAGCGCCA | |
| | GCGTGGGCGAGACAGTGACCATGACCTGTAGAGCCTCCGAGAATA | |
| | TCTACAGCTACCTGGCCTGGTACCAGCAGAAACAGGGCAAGAGCC | |
| | CCCAGCTGCTGGTCTTTAACGCCGAGACACTGCCTGAAGGCGTGC | |
| | CATCTAGATTCTCCGGCACCGGCAGCGGCACACACTTCAGCCTCC | |
| | GGATCAACAGCCTGCAACCTGAGGACTTCGGATCTTATTACTGCC | |
| | AGCACCACTACGTGATCCCCTGGACCTTCGGCGGAGGCACCAAGC | |
| | TGGAAATCAAG | |
| VSIG2 Ab | GATATCCAGATGACCCAGAGCCCTGCTTCTCTGAGCGCCAGCGTG | 84 |
| scFv (VL- | GGCGAGACAGTGACCATGACCTGTAGAGCCTCCGAGAATATCTAC | |
| (Whitlow)- | AGCTACCTGGCCTGGTACCAGCAGAAACAGGGCAAGAGCCCCCA | |
| VH) | GCTGCTGGTCTTTAACGCCGAGACACTGCCTGAAGGCGTGCCATC | |
| DNA Seq 2 | TAGATTCTCCGGCACCGGCAGCGGCACACACTTCAGCCTCCGGAT | |
| (SB04750) | CAACAGCCTGCAACCTGAGGACTTCGGATCTTATTACTGCCAGCA | |
| | CCACTACGTGATCCCCTGGACCTTCGGCGGAGGCACCAAGCTGGA | |
| | AATCAAGGGCAGCACAAGCGGCTCTGGAAAACCTGGATCTGGCG | |
| | AGGGCTCTACCAAGGGCGAGGTGCAGATGGTGGAAAGCGGCGGA | |
| | GATCTGGTGAAGCCTGGCGGCAGCCTGAAGCTGTCTTGTGCCGCT | |
| | TCTGGCTTTACCTTCAGCAACAGCGGCATGAGCTGGGTCAGACAG | |
| | ACCCCTGATAAGCGGCTGGAATGGGTGGCCAGCATCAGCGACGGC | |
| | GGACTGTACACCCACTACCCAGACAGCGTGAAAGGCAGATTCACA | |
| | ATCAGCCGGGACAACGGCAAGTCCACCCTGTATCTGCAGATGTCT | |
| | AGCCTGAGAAGCGAGGACACCGCCATCTACTACTGCGCCAGACAA | |
| | GGCGTGCGCCCCTTCTTCGACTACTGGGGCCAGGGAACAACACTC | |
| | ACCGTGTCCTCC | |

### Activating CAR NK-Cell Assays

Primary NK cells are isolated from human donor PBMCs and frozen. NK cells are transduced with a CAR lentivirus containing a selected activating CAR (aCAR) as described in Table C.

Killing assays are conducted to assess killing of VSIG-2 expressing cells, including HT29 cells, which are a human colorectal adenocarcinoma cell line that endogenously expresses VSIG2; Ls174t cells exogenously expressing VSIG2; and DLD1 cells exogenously expressing VSIG2. The killing assays will demonstrate that NK cells expressing an anti-VSIG2 aCAR are capable of killing a VSIG2-expressing cells.

### Example 3: Generation of Anti-VSIG2 Inhibitory CARs

Anti-VSIG2 inhibitory CAR (iCAR) constructs were each cloned into a lentiviral vector. The antigen specificity and domain organization for the CAR constructs are described in Table E below. The amino acid sequence for each anti-VSIG2 scFv of the iCAR constructs is shown above in Table C. The nucleotide sequence for each anti-VSIG2 scFv is shown in Table F below.

TABLE E
Inhibitory CAR Constructs

| Construct | Description |
|---|---|
| SB04751 | VSIG2 iCAR #1 |
| SB04752 | SIG2 iCAR #2 |
| SB04753 | SIG2 iCAR #3 |
| SB04754 | SIG2 iCAR #4 |

To test for the inhibitory activity of NOT-Gate anti-VSIG2 iCARs, individual anti-VSIG2 iCAR and anti-CEA aCAR constructs are packaged into lentiviral particles and used to transduce primary NK cells. Virus amounts are set by p24 titer (750,000 pg per transduction). iCAR constructs contained puroR cassettes, so puromycin is added to NK cell cultures from day 4 to 7 post transduction, at which time expression is assessed by flow cytometry and NK cells were transferred to a microwell plate for killing assays. NK cells are cultured with (1) tumor cells expressing aCAR antigen CEA only, (2) tumor cells expressing aCAR antigen CEA and iCAR antigen VSIG2, or (3) both tumor cell types mixed. After 16-18 hrs, cultures are analyzed by flow cytometry and remaining live targets cells of each type are counted. aCAR-mediated killing (basal subtracted) of a given NK cell type is quantified by first calculating total killing (reduction of targets compared to a target-only condition), and then subtracting total killing by control (iCAR-only) NK cells. iCAR-mediated protection is quantified as the change in aCAR-mediated killing between targets with or without iCAR antigen. Killing assay supernatant is analyzed for TNFα secretion, and aCAR and iCAR performance metrics are calculated analogously to killing. For expression analysis, iCARs and aCARs are stained for their respective epitope tag. The results will demonstrate inhibitory activity of the NOT-Gate anti-VSIG2 iCARs.

### Example 4: NOT Gate-Based Protection of Safety Antigen-Positive Cells

In this example, use of a NOT-Gate in combination with a CEA activating CAR was assessed using cell expressing a safety antigen (VSIG2).

First, cell lines expressing the safety antigen (VSIG2) were generated. SEM cells were transduced using lentivirus expressing the VSIG2 protein and a resistance selection marker (blasticidin). Cells were selected for antibiotic resistance and expression of the desired protein was assessed via flow cytometry. The SEM cells also transduced with lentivirus encoding for a membrane-bound form of CEACAM5-EGFP. Cells were sorted for EGFP positive and co-expression of CEACAM5 and VSIG2 was determined via flow cytometry.

Next, NK cells expressing a CEA activating CAR and an anti-VSIG2 inhibitory CAR were generated. Various anti-VSIG2 inhibitory CARs having various inhibitory domains (those with multiple intracellular domains are listed in order of proximity to the transmembrane domain: LIR1-LIR1, KIR3DL1, KIR3DL1-KIR3DL1, LIR1-KIR3DL1, KIR3DL1-LIR1, KIR2DL1, LAIR1, SIGLEC2, and SIRPa). For each with only a single intracellular domain, a transmembrane domain from the same protein was used. For those with two intracellular domains, a transmembrane domain from the same protein as the first intracellular domain was used. Additionally, each CAR included a CD8 hinge, and a V5 epitope tag between the scFv and the hinge. Construct descriptions are provided in Table F.

TABLE F
Description of Anti-VSIG2 Inhibitory
CAR Domains and Organization

| | CAR Description |
|---|---|
| | (scFv -TM - ICD - linker - |
| Construct Name | resistance marker) |
| “LIR1-LIR1” | aVSIG2 (L-w-H) - |
| | LIR1-LIR1 2A PuroR |
| “KIR3DL1” | aVSIG2 (L-w-H) - |
| | KIR3DL1 2A PuroR |
| “KIR3DL1-KIR3DL1” | aVSIG2 (L-w-H) - |
| | KIR3DL1-KIR3DL1 |
| | 2A PuroR |
| “LIR1-KIR3DL1” | aVSIG2 (L-w-H) - |
| | LIR1-KIR3DL1 2A |
| | PuroR |
| “KIR3DL1-LIR1” | aVSIG2 (L-w-H) - |
| | KIR3DL1-LIR1 2A |
| | PuroR |
| “KIR2DL1” | aVSIG2 (L-w-H) - |
| | KIR2DL1 2A PuroR |
| “LAIR1” | aVSIG2 (L-w-H) - |
| | LAIR1 2A PuroR |
| “SIGLEC-2” | aVSIG2 (L-w-H) - |
| | SIGLEC-2 2A PuroR |
| “SIRPa” | aVSIG2 (L-w-H) - |
| | SIRPa 2A PuroR |

Components of the anti-VSIG2 inhibitory CARs are provided in Table G.

TABLE G
Anti-VSIG2 inhibitory CAR Components and Seqeunces

Anti-VSIG2 scFv Sequence
DIQMTQSPASLSASVGETVTMTCRASENIYSYLAWYQQKQGKSPQLLVFNAETLPE
GVPSRFSGTGSGTHFSLRINSLQPEDFGSYYCQHHYVIPWTFGGGTKLEIKGSTSGSG
KPGSGEGSTKGEVQMVESGGDLVKPGGSLKLSCAASGFTFSNSGMSWVRQTPDKR
LEWVASISDGGLYTHYPDSVKGRFTISRDNGKSTLYLQMSSLRSEDTAIYYCARQG
VRPFFDYWGQGTTLTVSS (SEQ ID NO: 87)
LAIR1 TM Domain
ILIGVSVVFLFCLLLLVLFCL (SEQ ID NO: 88)
LAIR1 Intracellular Doman
HRQNQIKQGPPRSKDEEQKPQQRPDLAVDVLERTADKATVNGLPEKDRETDTSAL
AAGSSQEVTYAQLDHWALTQRTARAVSPQSTKPMAESITYAAVARH (SEQ ID
NO: 89)
LIR1 TM Domain
VIGILVAVILLLLLLLLLFLI (SEQ ID NO: 90)
LIR1 Intracellular Domain
LRHRRQGKHWTSTQRKADFQHPAGAVGPEPTDRGLQWRSSPAADAQEENLYAAV
KHTQPEDGVEMDTRSPHDEDPQAVTYAEVKHSRPRREMASPPSPLSGEFLDTKDRQ
AEEDRQMDTEAAASEAPQDVTYAQLHSLTLRREATEPPPSQEGPSPAVPSIYATLAI
H (SEQ ID NO: 91)
KIR2DL1 TM
ILIGTSVVIILFILLFFLL (SEQ ID NO: 92)
KIR2DL1 Intracellular Domain
HRWCSNKKNAAVMDQESAGNRTANSEDSDEQDPQEVTYTQLNHCVFTQRKITRPS
QRPKTPPTDIIVYTELPNAESRSKVVSCP (SEQ ID NO: 93)
SIGLEC2 TM
VAVGLGSCLAILILAICGL (SEQ ID NO: 94)
SIGLEC2 Intracellular Domain
KLQRRWKRTQSQQGLQENSSGQSFFVRNKKVRRAPLSEGPHSLGCYNPMMEDGIS
YTTLRFPEMNIPRTGDAESSEMQRPPPDCDDTVTYSALHKRQVGDYENVIPDFPEDE
GIHYSELIQFGVGERPQAQENVDYVILKH (SEQ ID NO: 95)
SIRPa TM Domain
IVVGVVCTLLVALLMAALYL (SEQ ID NO: 96)
SIRPa Intracellular Domain
VRIRQKKAQGSTSSTRLHEPEKNAREITQDTNDITYADLNLPKGKKPAPQAAEPNNH
TEYASIQTSPQPASEDTLTYADLDMVHLNRTPKQPAPKPEPSFSEYASVQVPRK
(SEQ ID NO: 97)
KIR3DL1 TM Domain
ILIGTSVVIILFILLLFFLL (SEQ ID NO: 98)
KIR3DL1 Intracellular Domain
HLWCSNKKNAAVMDQEPAGNRTANSEDSDEQDPEEVTYAQLDHCVFTQRKITRPS
QRPKTPPTDTILYTELPNAKPRSKVVSCP (SEQ ID NO: 99)
CD8 Hinge
TTTPAPRPPTPAPTIALQPLSLRPEACRPAAGGAVHTRGLDFACD (SEQ ID NO: 100)

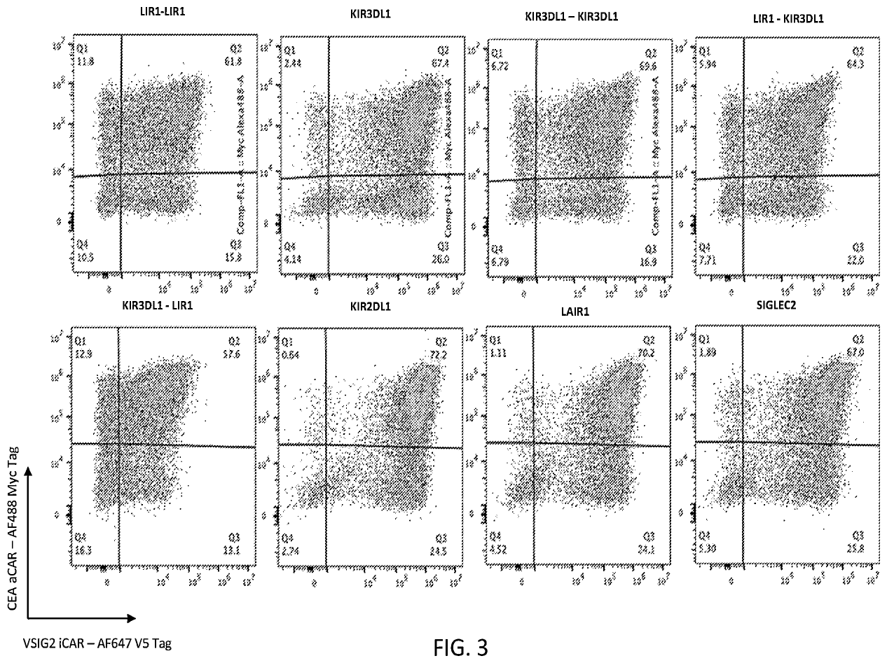

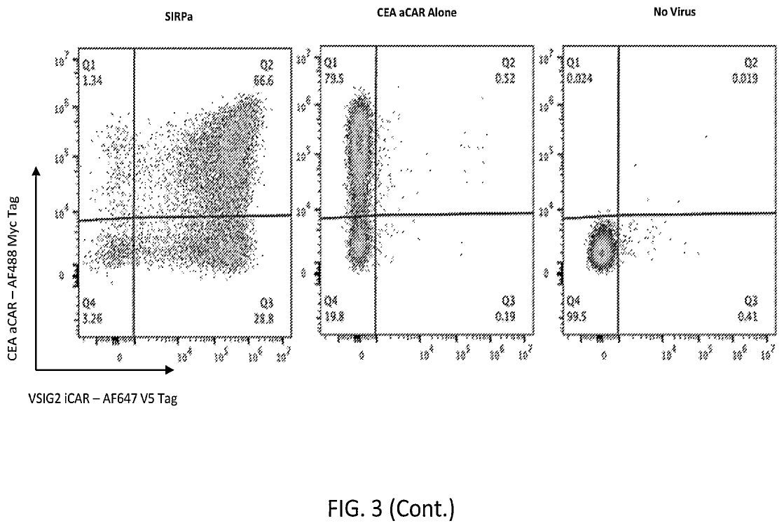

Primary, donor-derived NK cells were first expanded and then transduced with retrovirus encoding the CARs. Expression of aCAR was determined via flow cytometry using a MYC tag. Expression for the VSIG2 safety antigen system is shown in FIG. 3.

NK cells expressing a CEA-aCAR and various anti-VSIG2 iCARs were co-cultured with target cells that expressed either CEACAM5 only or CEACAM5/Safety antigen (VSIG2). Appropriate controls such as aCAR alone, iCAR alone and non-targeting iCAR were used.

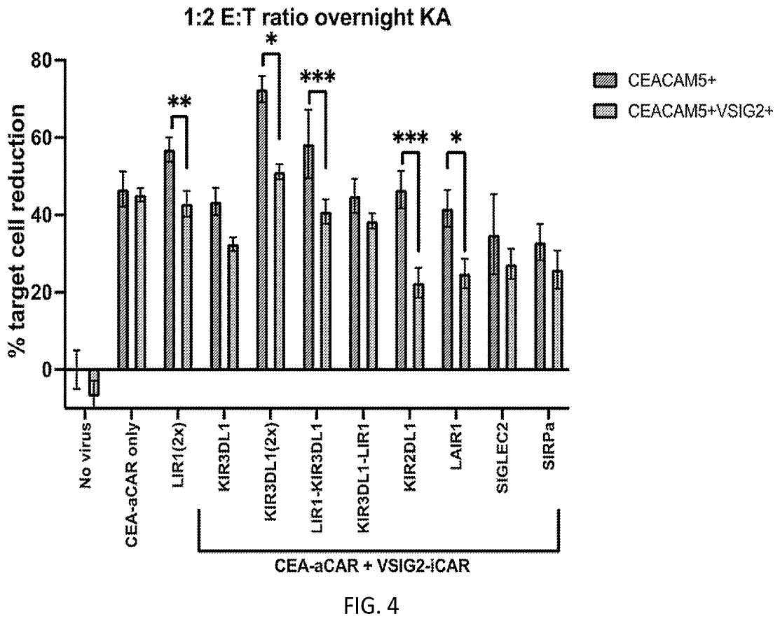

Percent target cell reduction was measured after an overnight co-culture with NOT-gated CAR NK cells using flow cytometry as shown in FIG. 4. As can be seen in FIG. 4, various VSIG2-inhibitory CARs containing various inhibitory domains show significant reduction of CAR-mediated cell killing in target cells that express the safety antigen (VSIG2, blue bars) and not in target cells that express only CEACAM5 (red bars). This result indicates that expression of a VSIG2 inhibitory CAR reduces activating CAR-mediated signaling in a safety-antigen dependent manner.

### Example 5: Characterization of VSIG2 Binders and Use as a Safety Antigen

In this example, CARs comprising the VSIG2 binders described in the above Examples were analyzed for activity as activating CARs (aCAR) and inhibitory CARs (iCAR).

A summary of the VSIG2 CARs used in this Example is provided in Table H below. Corresponding sequences are provided in Example 3 above.

TABLE H
Summary of the VSIG2 CAR Constructs Assessed

| | Vector | | Signal | | scFv | | Epitope | | TM | | |
|---|---|---|---|---|---|---|---|---|---|---|---|
| SB ID | Backbone | Promoter | Sequence | scFv | Orientation | Linker | Tag | Hinge | Domain | ICD 1 | ICD 2 |
| SB04741 | Sinvec | SFFV | CD8 | aVSIG2 | H/L | (G4S)3 | V5 | CD8 | CD28 | CD28 | CD3z |
| | | | | | | (SEQ | | | | | |
| | | | | | | ID NO: | | | | | |
| | | | | | | 31) | | | | | |
| SB04742 | Sinvec | SFFV | CD8 | aVSIG2 | L/H | (G4S)3 | V5 | CD8 | CD28 | CD28 | CD3z |
| | | | | | | (SEQ | | | | | |
| | | | | | | ID NO: | | | | | |
| | | | | | | 31) | | | | | |
| SB04743 | Sinvec | SFFV | CD8 | aVSIG2 | H/L | Whitlow | V5 | CD8 | CD28 | CD28 | CD3z |
| SB04744 | Sinvec | SFFV | CD8 | aVSIG2 | L/H | Whitlow | V5 | CD8 | CD28 | CD28 | CD3z |
| SB04745 | Sinvec | SFFV | CD8 | aVSIG2 | H/L | (G4S)1 | V5 | CD8 | CD28 | CD28 | CD3z |
| | | | | | | (SEQ | | | | | |
| | | | | | | ID NO: | | | | | |
| | | | | | | 29) | | | | | |
| SB04746 | Sinvec | SFFV | CD8 | aVSIG2 | L/H | (G4S)1 | V5 | CD8 | CD28 | CD28 | CD3z |
| | | | | | | (SEQ | | | | | |
| | | | | | | ID NO: | | | | | |
| | | | | | | 29) | | | | | |
| SB04747 | Sinvec | SFFV | CD8 | aVSIG2 | H/L | (G4S)3 | V5 | CD8 | CD28 | CD28 | CD3z |
| | | | | | | SEQ | | | | | |
| | | | | | | ID NO: | | | | | |
| | | | | | | 31) | | | | | |
| SB04748 | Sinvec | SFFV | CD8 | aVSIG2 | L/H | (G4S)3 | V5 | CD8 | CD28 | CD28 | CD3z |
| | | | | | | (SEQ | | | | | |
| | | | | | | ID NO: | | | | | |
| | | | | | | 31) | | | | | |
| SB04749 | Sinvec | SFFV | CD8 | aVSIG2 | H/L | Whitlow | V5 | CD8 | CD28 | CD28 | CD3z |
| SB04750 | Sinvec | SFFV | CD8 | aVSIG2 | L/H | Whitlow | V5 | CD8 | CD28 | CD28 | CD3z |

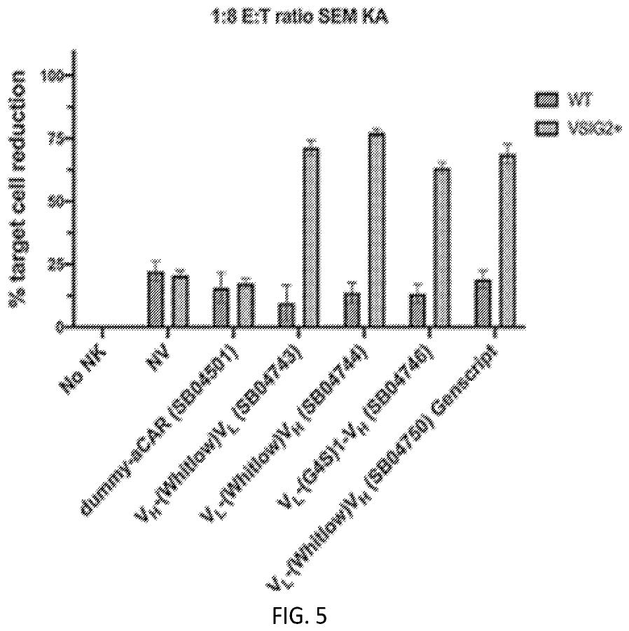

VSIG2 scFv sequences were used to build CD28z aCARs using different linkers and orientations to confirm expression, binding, and killing of VSIG2-positive target cells by NK cells. NK cells were transduced with retrovirus expressing different VSIG2-aCARs (as shown in Table H above). Different VSIG2 binders were designed based on the murine sequence of monoclonal VSIG2 antibody and built as activating CAR's with CD28 ICD and CD3z signaling domains. Transduced NK cells were co-cultured with SEM target cells engineered to overexpress human VSIG2, using methodologies as described in Example 4 above, at an effector cell:target cell (E:T) ratio of 1:8. Target cell death was quantified in comparison to wildtype (no VSIG2 expressing) target cells. Results are shown in FIG. 5.

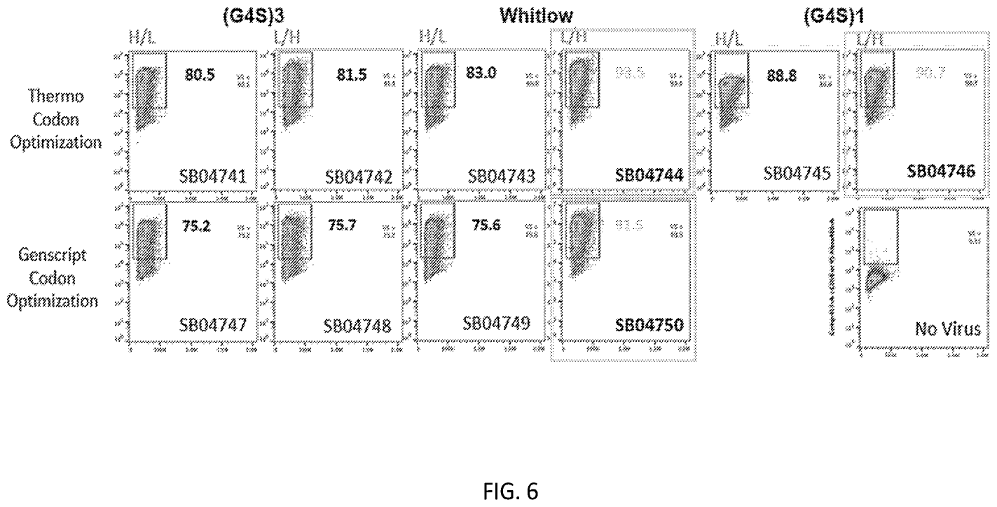

On Day 6 post-transduction, expression of VSIG2 aCARs with different binders was determined (FIG. 6). It was observed that the VL/VH orientation yielded higher mean fluorescence intensity (MFI) than VH/VL orientation of the scFvs. The highest expression was observed for SB04750, SB04746, and SB04744.

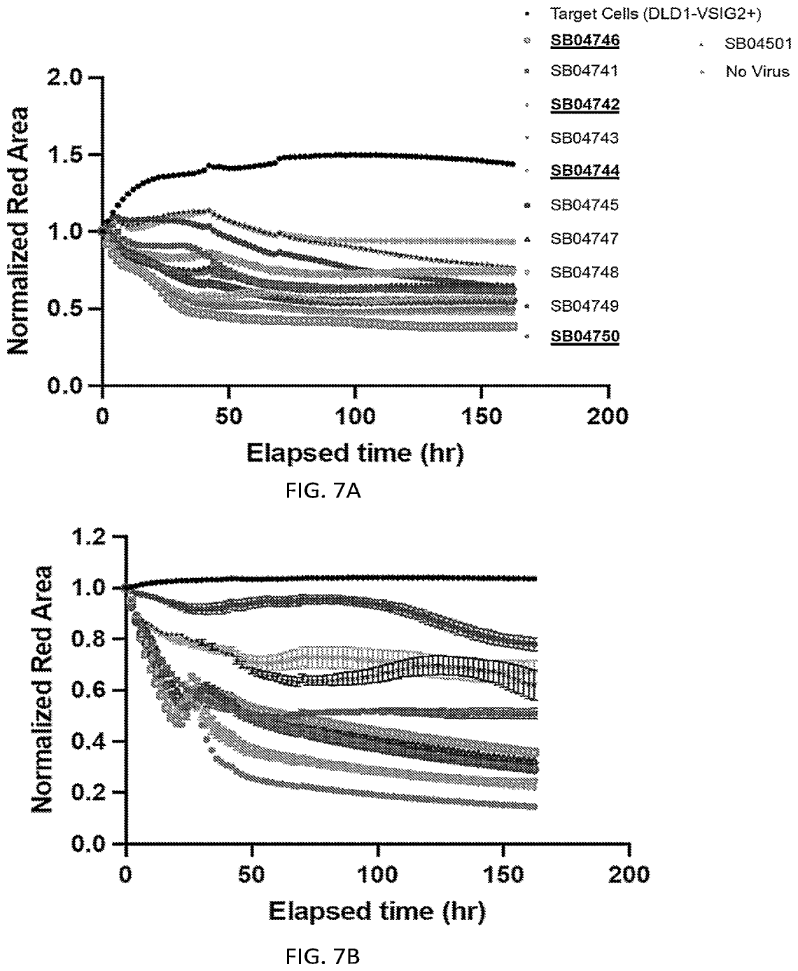

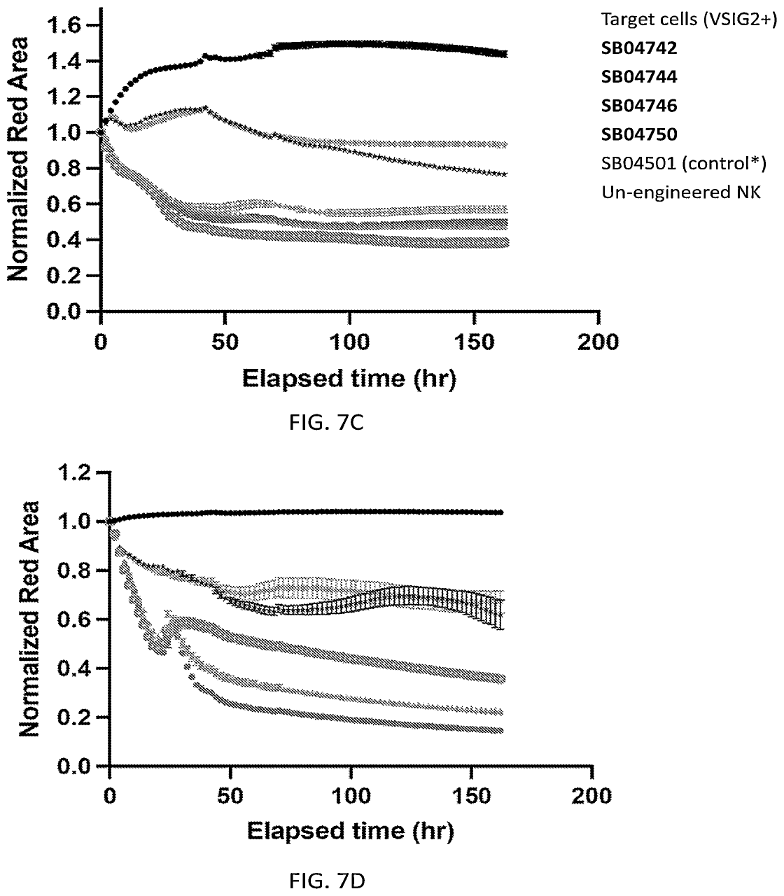

VSIG2-CAR NK cells were co-cultured with colorectal cancer (CRC) target cells expressing mKate and VSIG2 (Ls174t, FIG. 7A and DLD1, FIG. 7B). Target cell growth was measured on Incucyte® via red fluorescence (mKATE). All VSIG2 aCARs tested showed increased killing as compared to untransduced (NV) and control (SB04501, a non-targeting aCAR without an scFv). The highest killing was observed for SB04750, SB04746, and SB04744. Killing by selected scFvs is further shown in FIG. 7C (Ls174t cells) and FIG. 7D (DLD1 cells).

### VSIG2 as FLT3 NOT GATE

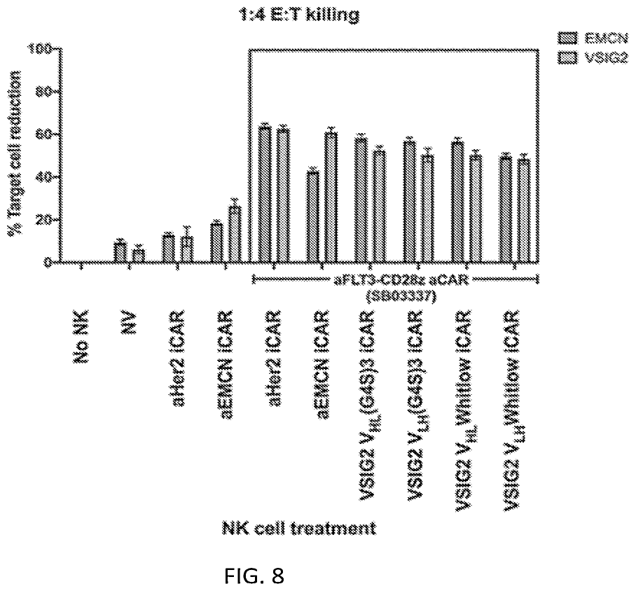

Use of VSIG2 iCARs as FLT3 NOT GATES was then tested. NK cells were co-cultured with SEM target cells that overexpress FLT3 (generated using metholodogies described above) as compared to target cells that express both FLT3 and VSIG2 at a 1:4 E:T ratio. NK cells were transduced with retrovirus expressing a FLT3-aCAR and VSIG2-iCARs with different binders. FIG. 8 shows VSIG2 CARs displaying increased killing over both target cell lines.

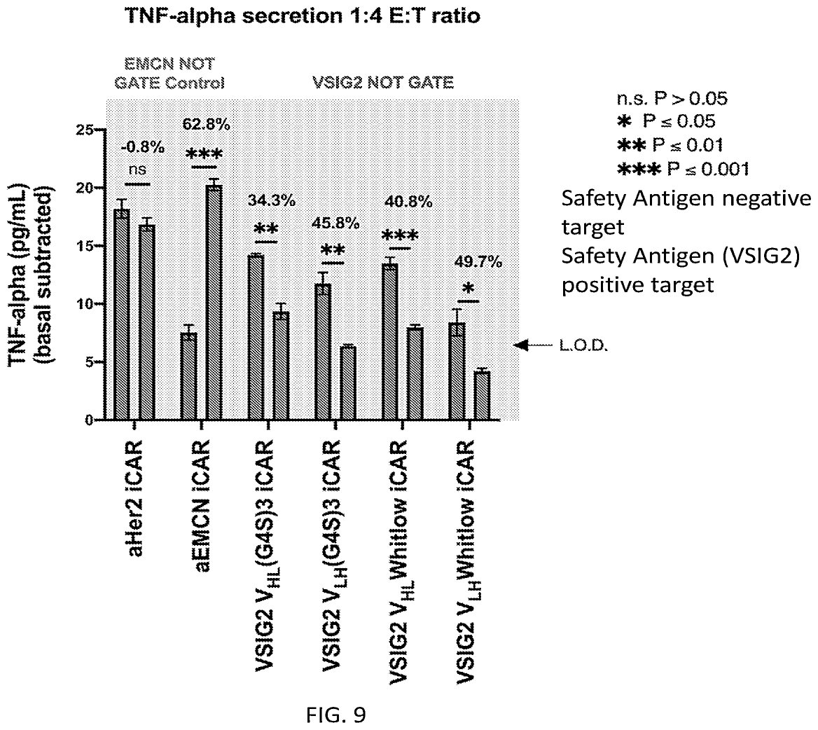

Supernatants from a co-culture experiment with NK cells and FLT3-target cells were then analyzed using Luminex to quantify cytokine production. TNFα production was reduced when NOT-gated CAR-NK cells were co-cultured with VSIG2-positive target cells, as shown in FIG. 9.

### VSIG2 as CEA NOT GATE

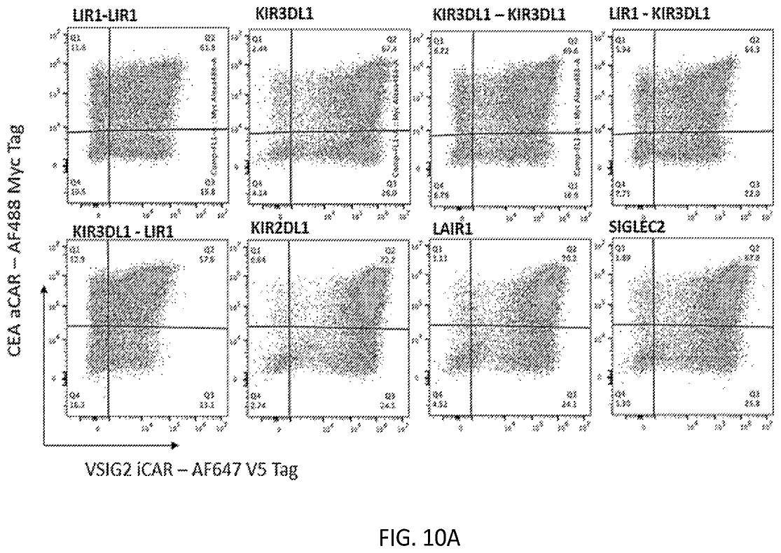

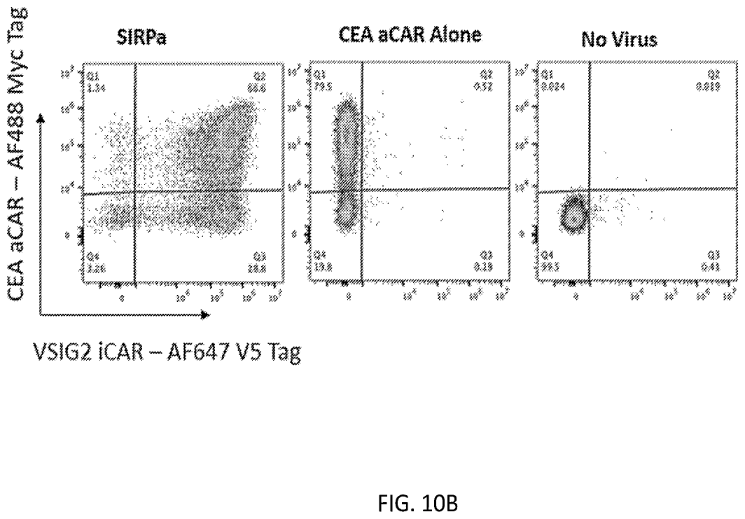

NK cells were transduced with retrovirus expressing CEA-aCAR and various VSIG2-iCARs that were built using the selected VSIG2 binders, and flow cytometry was used to determine expression of CEA-aCAR and VSIG2-iCAR on NK cells (FIG. 10A, control lines shown in FIG. 10B).

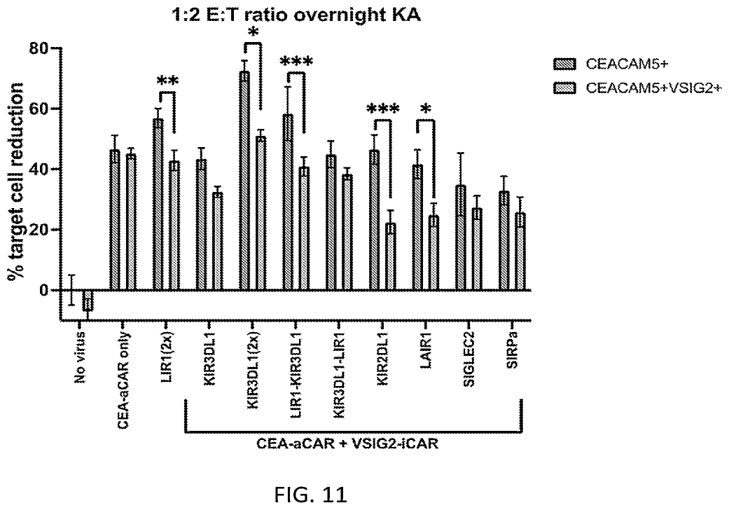

Transduced NK cells were then co-cultured with target cells engineered to overexpress human VSIG2 at a 1:2 E:T ratio. Target cell reduction was compared to target cells not expressing VSIG2 (left columns). Importantly, several VSIG2 iCARS significantly reduced NK-mediated killing of target cells in a VSIG2-dependent manner (shown in FIG. 11).

These data demonstrate that the tested VSIG2 scFvs may be used as safety antigens, for example, for NOT GATE technology in combination with an activating CAR.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

### OTHER SEQUENCES

Other sequences related to the resent disclosure are resented below:

| | SEQ | |
|---|---|---|
| Sequence | ID NO: | Description |
| GGAGGCGGAGGATCTGGTGGCGGAGGAAGTGGCGGAGGC | 85 | (G4S)₃ linker (SEQ ID |
| GGTTCT | | NO: 31) nucleic acid |
| GGTGGTGGTGGCAGTGGTGGCGGTGGCTCAGGTGGCGGCG | 86 | (G4S)₅ linker (SEQ ID |
| GATCAGGCGGTGGTGGTTCTGGCGGCGGTGGATCT | | NO: 102) nucleic acid |

1. A chimeric protein comprising an antigen-binding domain specific for V-Set And Immunoglobulin Domain Containing 2 (VSIG2) and a heterologous molecule or moiety,
wherein the antigen-binding domain comprises a heavy chain variable (VH) region and a light chain variable (VL) region, wherein
(a) the VH comprises:
a heavy chain complementarity determining region 1 (CDR-H1) having the amino acid sequence of GFTFSNS (SEQ ID NO:2),
a heavy chain complementarity determining region 2 (CDR-H2) having the amino acid sequence of SDGGLY (SEQ ID NO:3), and
a heavy chain complementarity determining region 3 (CDR-H3) having the amino acid sequence of QGVRPFFDY (SEQ ID NO:4), and
the VL comprises:
a light chain complementarity determining region 1 (CDR-L1) having the amino acid sequence of RASENIYSYLA (SEQ ID NO: 11) or RASENLYSYLA (SEQ ID NO:12),
a light chain complementarity determining region 2 (CDR-L2) having the amino acid sequence of NAETLPE (SEQ ID NO:13), and
a light chain complementarity determining region 3 (CDR-L3) having the amino acid sequence of QHHYVIPWT (SEQ ID NO:14); or
(b) the VH comprises:
a heavy chain complementarity determining region 1 (CDR-H1) contained within the VH region amino acid sequence of SEQ ID NO: 1, a heavy chain complementarity determining region 2 (CDR-H2) contained within the VH region amino acid sequence of SEQ ID NO: 1, and a heavy chain complementarity determining region 3 (CDR-H3) contained within the VH region amino acid sequence of SEQ ID NO: 1, and
the VL comprises:
a light chain complementarity determining region 1 (CDR-L1) are contained within the VL region amino acid sequence of SEQ ID NO: 9, a light chain complementarity determining region 2 (CDR-L2) are contained within the VL region amino acid sequence of SEQ ID NO: 9, and a light chain complementarity determining region 3 (CDR-L3) are contained within the VL region amino acid sequence of SEQ ID NO: 9; or
(c) the VH comprises:
a heavy chain complementarity determining region 1 (CDR-H1) contained within the VH region amino acid sequence of SEQ ID NO: 1, a heavy chain complementarity determining region 2 (CDR-H2) contained within the VH region amino acid sequence of SEQ ID NO: 1, and a heavy chain complementarity determining region 3 (CDR-H3) contained within the VH region amino acid sequence of SEQ ID NO: 1, and
the VL comprises:
a light chain complementarity determining region 1 (CDR-L1) are contained within the VL region amino acid sequence of SEQ ID NO: 10, a light chain complementarity determining region 2 (CDR-L2) are contained within the VL region amino acid sequence of SEQ ID NO:10, and a light chain complementarity determining region 3 (CDR-L3) are contained within the VL region amino acid sequence of SEQ ID NO: 10, and
optionally wherein the amino acid sequences of the CDR-H1, the CDR-H2, the CDR-H3, the CDR-L1, the CDR-L2, and the CDR-L3 of the reference antibody are defined based on the Kabat or Chothia numbering scheme.

2. The chimeric protein of claim 1, wherein the VH region comprises the amino acid sequence of SEQ ID NO: 1.

3. The chimeric protein of claim 1, wherein the VL region comprises the amino acid sequence of SEQ ID NO: 9, or the VL region comprises the amino acid sequence of SEQ ID NO: 10.

4. The chimeric protein of claim 1, wherein the antigen-binding domain comprises a single chain variable fragment (scFv),
optionally wherein the VH and VL of the scFv are separated by a peptide linker,
optionally wherein the antigen-binding domain comprises the structure VH-L-VL or VL-L-VH,
wherein VH is the heavy chain variable domain, L is the peptide linker, and VL is the light chain variable domain, and/or
optionally wherein the scFv comprises an amino acid sequence selected from the group consisting of: SEQ ID Nos: 19-35 and 69-74.

5. The chimeric protein of claim 1, wherein the chimeric protein is a chimeric antigen receptor (CAR), and wherein the heterologous molecule or moiety comprises a polypeptide selected from the group consisting of: a transmembrane domain, one or more intracellular signaling domains, a hinge domain, a spacer region, one or more peptide linkers, and combinations thereof.

6. The chimeric protein of claim 5, wherein the CAR is an inhibitory CAR comprising one or more intracellular inhibitory domains that inhibit an immune response and wherein each of the one or more intracellular inhibitory domains comprises an enzymatic inhibitory domain or an intracellular inhibitory co-signaling domain.

7. An engineered polynucleotide encoding the chimeric protein of claim 1.

8. An expression vector comprising the engineered polynucleotide of claim 7.

9. An isolated cell or a population of engineered cells comprising the chimeric protein of claim 1.

10. (canceled)

11. The cell or the population of engineered cells of claim 9, wherein the cell or population of cells further comprises one or more tumor-targeting chimeric receptors expressed on the cell surface, optionally wherein each of the one or more tumor-targeting chimeric receptors is a chimeric antigen receptor (CAR) or an engineered T cell receptor.

12. The cell or the population of engineered cells of claim 9, wherein the cell or the population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

13. A pharmaceutical composition comprising an effective amount of the cell or the population of engineered cells of claim 9 and a pharmaceutically acceptable carrier, pharmaceutically acceptable excipient, or a combination thereof.

14. A method of stimulating a cell-mediated immune response to a tumor cell in a subject, the method comprising administering to a subject having a tumor a therapeutically effective dose of the chimeric protein of claim 1.

15. A method of treating a subject having a tumor, the method comprising administering a therapeutically effective dose of the chimeric protein of claim 1.

16. A method of treating a subject having a tumor, the method comprising administering a therapeutically effective dose of the cell or the population of cells of claim 9.

17. A method of treating a subject having a tumor, the method comprising administering a therapeutically effective dose of the pharmaceutical composition of claim 13.

18. A method of stimulating a cell-mediated immune response to a tumor cell in a subject, the method comprising administering to a subject having a tumor a therapeutically effective dose of the chimeric protein of the cell or the population of cells of claim 9.

19. A method of stimulating a cell-mediated immune response to a tumor cell in a subject, the method comprising administering to a subject having a tumor a therapeutically effective dose of the chimeric protein of claim 13.
